(19) United States
(12) Patent Application Publication
(10) Pub. No.: US 2023/0004026 A1
(43) Pub. Date: Jan. 5, 2023

(54) OPHTHALMIC LENS DESIGNS WITH NON-REFRACTIVE FEATURES

(71) Applicant: NTHALMIC HOLDING PTY LTD, Sydney, New South Wales (AU)

(72) Inventors: Ravi Chandra Bakaraju, Sydney, New South Wales (AU); Darrin Falk, Sydney, New South Wales (AU); Klaus Ehrmann, Sydney, New South Wales (AU)

(21) Appl. No.: 17/780,484

(22) PCT Filed: Nov. 30, 2020
(86) PCT No.: PCT/AU2020/051296

§ 371 Date: May 26, 2022

(30) Foreign Application Priority Data
Dec. 1, 2019 (AU) 2019904536
Dec. 1, 2020 (AU) 2019904537

(51) Int. Cl. G02C 7/10

(52) U.S. Cl. CPC G02C 7/10; G02C 2202/24

(57) ABSTRACT

The present disclosure relates to use of single vision ophthalmic lenses for correction of myopia in a wearer, wherein the single vision ophthalmic lens devices are configured with a base prescription to correct the myopia of the individual and are purposefully further configured with non-refractive features, wherein the non-refractive features facilitate an increase in the retinal ganglion cell activity for the wearer, which may serve as an optical stop signal to decelerate, ameliorate, control, inhibit, or reduce, the rate of myopia progression of the wearer.

### CROSS-REFERENCE

This patent application claims priority to the Australian Provisional Application serial no. 2019/904536, filed on Dec. 1, 2019, entitled “A multi-zone ophthalmic lens”; and another Australian Provisional Application serial no. 2019/904537, filed on Dec. 1, 2019, entitled “An Ophthalmic lens for myopia”; both of which are herein incorporated by reference in their entirety.

### FIELD OF THE INVENTION

The present disclosure relates to ophthalmic lenses, particularly to contact lenses and spectacle lenses, for use with eyes experiencing eye-length related disorders, like myopia.

### BACKGROUND

The human retina has three primary layers: the photoreceptor layer, the outer plexiform layer, and the inner plexiform layer. Cones and rods are the photoreceptors that respond to light in the retina of a human eye by transforming incoming light into electrical signals. The transduced electrical signal propagates from the photoreceptors, through the bipolar cells, further to the retinal ganglion cells and the optic nerve, carrying visual information from the retinal cells to the brain allowing visual perception of the world. The photoreceptors respond with graded membrane potentials and release neurotransmitter glutamate proportional to the level of their polarisation state. For example, in absence of a light stimulus, the photoreceptors depolarise and release more glutamate relative to its baseline state. In the presence of light, the photoreceptors hyperpolarise, which happens due to the breakdown of opsins in the photoreceptors, causing it to release less glutamate relative to its baseline state. There are two types of bipolar cells in the retina, on- and off-centre bipolar cells, which separately encode positive and negative spatiotemporal contrast from incoming light by comparing the photoreceptor signals to spatiotemporal averages computed by the laterally connected layer of horizontal cells.

The horizontal cells are interconnected by conductive gap junctions and are connected to bipolar cells and photoreceptors in a complex triad synapse. The on- and off-centre bipolar cells have different responses to glutamate, which are based on the type and number of glutamate receptors located on each of these bipolar cells.

The off-centre bipolar cells have ionotropic receptors, which are excitatory to glutamate. These off-centre bipolar cells depolarise in response to glutamate and preserve the sign of the photoreceptors' signal. In presence of light, off-centre bipolar cells receive less glutamate from the photoreceptors, causing hyperpolarisation and release less glutamate to the corresponding ganglion cells downstream. In absence of light, the off-centre bipolar cells receive more glutamate from the photoreceptors, causing depolarisation and release more glutamate to the corresponding ganglion cells downstream.

The on-centre bipolar cells have metabotropic receptors, which are inhibitory to glutamate. These on-centre bipolar cells hyperpolarise in response to glutamate and reverse the sign of the photoreceptors' signal. In the presence of light, the on-centre bipolar cells receive less glutamate from the photoreceptors, causing depolarisation and release more glutamate to the corresponding ganglion cells downstream. In the absence of light, the on-centre bipolar cells receive more glutamate from the photoreceptors, causing hyperpolarisation and release less glutamate to the corresponding ganglion cells downstream. The more glutamate the on- or off-centre bipolar cells release onto the corresponding ganglion cells downstream, the greater is the action firing potential of the ganglion cells. The opposite responses to light between the on-centre bipolar and off-centre bipolar cells are the key to the differential response to light and dark states. In addition, the depolarising signal activity of the on- and off-centre bipolar cells may be amplified or suppressed by the horizontal cells that connect the surrounding photoreceptors in the corresponding receptive fields.

The horizontal cells receive excitatory input from the photoreceptors and send out inhibitory feedback in return to the photoreceptors connected in the surrounding neighbourhood. Receptive fields are groups of photoreceptors that send inputs downstream to bipolar and ganglion cells in the retina.

The retinal receptive field can be described using concentric circular zones with a small circular central field and a broader circular field around the central field called the surround field. The receptive fields fall into two categories, namely, the off-centre with on-surround type receptive field and the on-centre with off-surround type receptive field. On- and off-centre receptive fields have different responses to light based on differences in bipolar cells.

Human eyes are hyperopic at birth, where the length of the eyeball is too short for the total optical power of the eye. As the person ages from childhood to adulthood, the eyeball continues to grow until the eye's refractive state stabilises. The growth of the eye is understood to be controlled by a feedback mechanism and regulated predominantly by the visual experience, to match the eye's optics with the eye length and maintain homeostasis. This process is referred to as emmetropisation. The signals that guide the emmetropisation process are initiated by the modulation of light energy received by the retina. The retinal image characteristics are monitored by a biological process that modulates the signal to start or stop, accelerate, or slow eye growth. This process coordinates between the optics and the eyeball length to achieve or maintain emmetropia. Derailing from this emmetropisation process results in refractive disorders like myopia. It is hypothesised that diminished retinal activity encourages eye growth, and contrarily increase in retinal activity inhibits eye growth.

The prevalence of myopia is increasing at alarming rates in many regions of the world, particularly in East Asia. In myopic individuals, the axial length of the eye is mismatched to the overall power of the eye, leading to distant objects being focussed in front of the retina.

A simple pair of negative single vision lenses can correct myopia. While such devices can optically correct the refractive error associated with eye length, they do not address the underlying cause of the excessive eye growth in myopia progression. Excessive eye length in high degrees of myopia is associated with significant vision threatening conditions like cataract, glaucoma, myopic maculopathy, and retinal detachment. Thus, there remains a need for specific optical devices for such individuals, that not only correct the underlying refractive error, but also prevent excessive eye lengthening or progression of myopia.

### Definitions

Terms, as used herein, are generally used by a person skilled in the art, unless otherwise defined in the following.

The term “myopic eye” means an eye that is either already experiencing myopia, is in the stage of pre-myopia, is at risk of becoming myopic, is diagnosed to have a refractive condition that is progressing towards myopia with or without astigmatism.

The term “progressing myopic eye” means an eye with established myopia that is diagnosed to be progressing, as gauged by either the change in refractive error of at least −0.25 D/year or the change in axial length of at least 0.1 mm/year.

The term “an eye at risk of becoming myopic” means an eye, which could be emmetropic or is low hyperopic at the time but has been identified to have an increased risk of becoming myopic based on genetic factors (e.g. both parents are myopic) and/or age (e.g. being low hyperopic at a young age) and/or environmental factors (e.g. time spent outdoors) and/or behavioural factors (e.g. time spent performing near tasks).

The term “optical stop signal” or “stop signal” means an optical signal or directional cue that may facilitate slowing, reversing, arresting, retarding, inhibiting, or controlling the growth of an eye and/or refractive condition of the eye.

The terms “spatially and temporally varying optical stop signal” or “spatially and temporally variant optical stop signal” mean an optical stop signal, provided at the retina, which changes with time and spatially across the retina of the eye.

The term “contact lens” means a finished contact lens, suitable to be fit on the cornea of a wearer to affect the optical performance of the eye.

The term “spectacle lens” may mean a finished or semi-finished blank lens. The term “standard single vision spectacle lens” or “commercially available single vision spectacles” or “standard spectacles” means spectacle lenses with a base prescription used to correct the underlying refractive error of the eye; wherein the refractive error may be myopia, with or without astigmatism.

The term “optical zone” or “optic zone” means the region on an ophthalmic lens (e.g. contact lens or spectacle lens) which has the prescribed optical effect. The optical zone comprise one or both of a front and back optic zone. The front and back optic zones mean anterior and posterior surface areas of a contact lens which contribute to the prescribed optical effect, respectively.

The term “optical centre” or “optic centre” means the geometric centre of the optical zone of an ophthalmic. The terms geometrical and geometric are essentially the same.

The term “optical axis” means the line passing through the optical centre and substantially perpendicular to the plane containing the edge of the ophthalmic lens.

The term or phrase “single vision optical zone” or “substantially single vision optics” or “substantially single vision profile” or “spherical optical zone” means that the optical zone has a uniform power distribution without substantial amounts of primary spherical aberration. The single vision optical zone may be further classified to include astigmatic component to correct the distance refractive error.

The term “model eye” may mean a schematic, raytracing, or a physical model eye.

The terms “Diopter”, “Dioptre” or “D” as used herein is the unit measure of dioptric power, defined as the reciprocal of the focal distance of a lens or an optical system, in meters, along an optical axis.

### SUMMARY

The detailed discussion on the prior art, and the subject matter of interest in general, is provided as the background of the present disclosure, to illustrate the context of the disclosed embodiments, and furthermore, to distinguish the advances contemplated by the present disclosure over the prior art. No material presented here should be taken as an acknowledgment that the material mentioned is previously disclosed, known, or part of common general knowledge, on the priority of the various embodiments and/or claims set forth in the present disclosure.

Briefly summarised, all prior art optical designs with refractive or phase-altering features used for managing myopic refractive error involve significant visual compromises, which are primarily precipitated due to the use of multifocal-like design features often considered in the field. Examples are described in U.S. Pat. Nos. 6,045,578, 7,025,460, 7,506,983, 7,401,922, 7,803,153, 8,690,319, 8,931,897, 8,950,860, 8,998,408.

A catalogue of solutions has been proposed in the optical field with amplitude altering features to improve depth of focus for general imaging systems. Examples are described in the papers written by: Mino and Okano, Applied Optics 1971, entitled “Improvement in the OTF of a defocused optical system through the use of shaded apertures”; Castaneda et al., Applied Optics 1989, entitled “Arbitrary high focal depth with a quasi-optimum real and positive transmittance apodizer”; Castaneda and Berriel-Valdos, published in Applied Optics 1990, entitled “Zone plate for arbitrary high focal depth”; and U.S. Pat. No. 5,965,330A, 857065562 and 8192022.

The disadvantages with amplitude altering solutions include reduced energy transmission at critical frequencies, poorer resolution relative to their phase-altering counterparts, and low light throughput.

On the contrary, the present disclosure is directed to the use of single vision ophthalmic lens designs purposefully configured with a plurality of non-refractive features which are aimed to provide an increase in retinal ganglion cell activity and to overcome one or more of the drawbacks of the prior art, as described herein.

Certain disclosed embodiments are directed towards modifying the incoming light through a contact lens or a spectacle lens that utilise a stop signal to decelerate the rate of myopia progression. More specifically, the present disclosure relates to use of single vision contact and spectacle lenses for correction of myopia in a wearer, wherein the single vision ophthalmic lens devices are configured with a base prescription to correct the myopia of the individual and are purposefully further configured with non-refractive features, wherein the non-refractive features facilitate an increase in the retinal ganglion cell activity for the wearer, which may serve as an optical stop signal for inhibiting, reducing, or controlling the rate of myopia progression of the wearer. In some embodiments, the optical stop signal may be configured to have spatiotemporal variations.

Certain disclosed embodiments include contact lenses and/or spectacle lenses for altering the properties of incoming light entering a human eye. Certain disclosed embodiments are directed to the configuration of contact lenses and/or spectacle lenses for correcting, managing, and treating refractive errors, for example myopia. Some embodiments are aimed to both correct the myopic refractive error and simultaneously provide an optical stop signal that discourages further eye growth or progression of myopia.

Certain embodiments relate to an apparatus, device, and/or a method capable of modifying incident light through an ophthalmic lens to provide an active rise in retinal ganglion cell activity for slowing eye growth in an individual. This may be accomplished through the configuration of certain non-refractive features, used in conjunction with single vision ophthalmic lenses, that are aimed at introduction of artificial edge pattern, or artificial luminous contrast profiles, imposed onto the central and/or peripheral retina. The artificial edge patterns, or artificial luminous contrast profiles, imposed on the retina, offer a spatial contrast profile across the on- and off-centre retinal fields across the retina. The artificially induced edges provide an increase in the retinal spiking activity, or ganglion cell firing activity, which is a surrogate measure of overall retinal activity. The current disclosure postulates that increased retinal ganglion cell activity may in turn provide an optical stop signal to a progressing myopic eye.

In some other embodiments of the present disclosure, the non-refractive features of the contact lens are configured such that the artificial edge patterns, or artificial spatial luminous contrast profiles, imposed on the retina are further configured to offer a temporal variation in the overall retinal ganglion cell activity.

Certain embodiments of the present disclosure involve one or more variations of the structural characteristics of the non-refractive features, used in conjunction with single vision ophthalmic lenses, both contact lenses and spectacle lenses, as disclosed herein. For example, the structural characteristics of the non-refractive features include one or more of the following: their opaqueness, their size, width, and shape, their method of application, their location of application, their distribution, their arrangement pattern and spanning area, on the ophthalmic lens.

The contemplated variations of the numerous structural characteristics of the non-refractive features provide a desired on-eye functional visual performance, while maintaining the potency of the ophthalmic lens embodiments to slow the progression of myopia, as disclosed herein. Certain embodiments of the present disclosure involve optimisation of the non-refractive features, including but not limited to, the following features: opaqueness, size, shape, plurality, pattern, location, and method of application, to provide a desired level of increase and/or a desired level of temporal variance in retinal ganglion cell activity without compromising the eye's resolution capabilities. For example, in some embodiments of the present disclosure, the one or more characteristics of the non-refractive features are configured on an otherwise single vision ophthalmic lens with a base prescription to correct the refractive error of the eye, wherein the embodiment ophthalmic lens when tested on a model eye, presented with a number of common visual scenes that may include scenes typical of environments and or behaviours thought to be associated with myopia development and/or progression, provides an increase in the retinal ganglion cell activity by about at least 1.25 times, at least 1.5 times, at least 1.75 times, at least 2 times, at least 2.5 times or at least 3 times the retinal ganglion cell activity of a single vision ophthalmic lens without the non-refractive features; wherein the retinal ganglion cell activity may include on-type cells, off-type cells or both on-type and off-type cells within the receptive fields. In some examples. the retinal ganglion cell activity may be within a local region, a plurality of local regions, or averaged across the desired retinal field. In some other embodiments, the ophthalmic lens tested on a model additionally provides a temporal variation in the retinal ganglion cell activity. In some examples, the retinal ganglion cell activity may be gauged by the retinal spike train analysis, while in some other examples, it may be gauged by the average retinal spike rate as a function of time. In certain other embodiments of the disclosure, the embodiment ophthalmic lens when tested on a model eye provides increased temporal variations, or fluctuations or oscillations to the retinal ganglion cell activity; wherein the temporal variations of the retinal ganglion cell activity may be represented as one or more of the following: non-monotonic fluctuations, quasi-sinusoidal variations, sinusoidal variations, periodic variations, aperiodic variations, aperiodic quasi-rectangular variations, rectangular variations, square-wave variations, or random variations in the retinal ganglion cell activity.

In some examples, specific types of visual stimuli may be used to elicit the retinal ganglion cell activity, for example, white-noise electrical stimulation, a sinusoidal variation in the visual stimuli, a checkerboard pattern, full-field flash stimuli, semi-field flash stimuli, full-field Gaussian noise, semi-field Gaussian noise, regional flash stimuli, regional Gaussian noise, etc. In some examples only coarse characterisation of neural response to the stimuli may be desirable; while in other examples, much finer characterisation of neural response to stimuli may be desirable. The stimuli used in this disclosure are considered only a representative means to demonstrate the workings of the disclosure and the choice of should not be construed as limiting the scope of the disclosure and/or the claims.

In some embodiments of the present disclosure, the opaqueness of the non-refractive features on an ophthalmic lens may be configured such that the feature absorbs at least 80%, at least 85%, at least 90%, at least 95%, at least 98%, at least 99%, or all of 100%, of light incident on the non-refractive feature. In some other embodiments of the present disclosure, the opaqueness of the non-refractive features on an ophthalmic lens may be configured such that the feature absorbs between 80% to 90%, or between 80% to 95%, or between 80% to 99% of light incident on the non-refractive feature.

In some embodiments of the present disclosure, the width of any one or more of the individual element of any of the non-refractive features may be configured such that the feature is at least 3, at least 4, at least 5, at least 6, at least 7, at least 8, at least 9, or at least 10 times of the average wavelength of light in the visible spectrum (i.e. 555 nm).

In some other embodiments of the present disclosure, the width of any individual element of any of the non-refractive feature may be configures such that the feature is between 3 and 5 times, or between 4 and 7 times, or between 5 and 9 times, or between 3 and 10 times, of the average wavelength of light in the visible spectrum (i.e. 555 nm). The lower limit on the choice of the width of any individual element of the non-refractive features to be substantially greater than the average wavelength of light in the visible spectrum is underpinned by the desirable outcome to avoid unwarranted diffractive effects about the edges of the non-refractive features disclosed herein.

In some embodiments, the width of any one or more of the individual elements of the non-refractive features on an ophthalmic lens may be configured such that the feature is no greater than 50 μm, or no greater than 75 μm, or no greater than 100 μm, or no greater than 150 μm, or no greater than 200 μm, or no greater than 250 μm, or no greater than 300 μm. The upper limit on the choice of the width/size of any individual element of the non-refractive features is underpinned by the desirable outcome of maintaining an adequate amount of light entering the eye that allows for minimal energy loss and thereby allowing for substantially no change in the resolution capability for the eye wearing the contemplated embodiments disclosed herein. In some embodiments, the upper limit on the choice of the width/size of any individual element of the non-refractive feature may be different between contact lens and spectacle lens embodiment, accounting for the vertex distance in the latter.

In some other embodiments of the disclosure, the non-refractive features may be customised based on the degree of myopia and the rate of progression such that the potency of the reduction in rate of progression may be balanced with the desired degree of compromise in visual performance that may be accepted by the wearer.

In certain embodiments of the present disclosure, the shape of any one or more of the individual elements of the non-refractive features that may be configured on an ophthalmic lens may be configured such that the feature is circular, hexagonal, octagonal, regular polygon, irregular polygon, line, triangle, dot-like, arc-like or any other random shapes disclosed herein.

In some other embodiments, the contemplated design features of the multiple apertures, segments, regions, or zones, may be circular, non-circular, semi-circular, annular, oval, rectangular, octagonal, hexagonal, or square in shape.

In certain embodiments of the present disclosure, the arrangement of the individual elements of the non-refractive features on a single vision contact lens may be configured such that the area spanned by all of the non-refractive features is within the 2 mm, or within the 2.5 mm, or within the 3 mm, or within the 3.5 mm, or within the 4 mm, or within the 4.5 mm, or within the 5 mm, or within the 6 mm central diameter of the optic zone of the single vision contact lens.

In certain embodiments of the present disclosure, the arrangement of the individual elements of the non-refractive features on a single vision spectacle lens may be configured such that the area spanned by all of the non-refractive features is within the 20 mm, or within the 25 mm, or within the 30 mm, or within the 35 mm, or within the 40 mm, or within the 45 mm, or within the 50 mm, or within the 60 mm central diameter of the optic zone of the single vision spectacle lens.

In some other examples of the present disclosure, the non-refractive features may be implemented within the central 30%, 35%, 40% 45%, 50%, 55%, or 60%, area of the optical zone of the single vision ophthalmic lens.

In some other examples of the present disclosure, the non-refractive features may be implemented within the peripheral 10%, 15%, 20% 25%, 30%, 35%, or 40% area of the optical zone of the single vision ophthalmic lens. The references to central or peripheral portions of the single vision ophthalmic lens is made from the optical centre of the ophthalmic lens.

In some other examples of the present disclosure, the non-refractive features may be implemented on one or more of the following locations: the front surface of the ophthalmic lens, the back surface of the ophthalmic lens, within the matrix of the material of the ophthalmic lens. In some embodiments, the method of implementation of the non-refractive features may be achieved via pad-printing or laser printing approaches as used in the routine development of cosmetic lenses.

In some embodiments of the present disclosure, the implemented non-refractive features may be arranged in form of multiple apertures, multiple zones, multiple regions, multiple segments essentially over an otherwise substantially single vision ophthalmic lens that may facilitate an increase in retinal ganglion cell activity serving as an optical stop signal for inhibiting, reducing, or controlling progressive myopic refractive error, as disclosed herein.

In other embodiments, the non-refractive features may be implemented via homogeneous media or heterogeneous media configured into the matrix of the ophthalmic lens. In some other embodiments, the implementation may involve photo-etching of a media on the surface or within the matrix, or other photographic process.

The present disclosure relates to an ophthalmic lens which alters the transmitted properties of the incoming light, creating distinct luminous contrast profiles (i.e. artificial edges) on the retina of the wearer. The alteration of the transmission properties of the eye is achieved, by employing a plurality of relatively lower transmission lines or striae, or alternatively, by employing non-refractive features arranged as multiple apertures, zones, segments, regions, or other patterns contemplated herein. The low transmission lines or striae or features may be configured on one or more of the locations on the ophthalmic lens: front surface of the lens, back surface of the lens, or may be embedded within the matrix of the ophthalmic lens. The low transmission lines, striae or features may be configured to be opaque, translucent, reflective, spectrally sensitive, polarisation sensitive, or absorbent. To implement the polarisation sensitive materials, various combinations of linear polarisation filters with or without quarter-wave plate retarders may be considered. In some other embodiments, the desired polarisation sensitive characteristics may be configured using specific lens materials, for example birefringent materials, coatings, or combination thereof.

The dimensional specifications of the low transmission features, for example, width of the non-refractive features can be adjusted in the lens design as desired, to increase the amount of light entering the eye, minimise visual artefacts while adequately configuring the ophthalmic lens for the desired refractive correction of the wearer's eye and maintaining, or providing, an adequate stop signal to the wearer's eye.

The current disclosure proposes use of non-refractive features to retard the progression of myopia. The use of non-refractive features facilitates embodiments that do not utilise any of the phase-altering approaches of positive defocus, positive spherical aberration, or any other variants, for example, bifocal, multifocal or extended depth of focus optical features.

The current disclosure proposes a method to retard the progression of myopia by introducing artificial edges, or luminous contrast profiles, into the retinal imagery captured while viewing through the ophthalmic lenses and providing an increase in the retinal ganglion cell activity which may inhibit further eye growth.

In some embodiments, the ophthalmic lenses may mean contact lenses, while in yet other embodiments, the ophthalmic lenses may mean spectacle lenses. In some embodiments of the disclosure contemplating spectacle lenses, incorporation of the non-refractive features may lead to poor cosmetical appearance of the spectacle lens which may be undesirable for the wearer. Additional material characteristics of the lens may be contemplated to mitigate the issue of poor cosmesis. For example, in some embodiments, the implemented non-refractive features may be configured to have one or more of the following additional material properties: completely insensitive, partially sensitive, or fully sensitive, to the polarisation state of the incident light. In some other spectacle lens embodiments of the present disclosure, implemented non-refractive features may be configured to be electrically tuneable. In some embodiments, a combination of pair of polarised contact lens and pair of polarised spectacle lenses may be contemplated to offer additional temporal variation in retinal ganglion cell activity without requiring excessive movement of contact lenses on eye.

Certain embodiments of the current disclosure includes a contact lens that is purposefully designed with non-refractive features arranged in, for example, a moiré pattern, curvilinear pattern, Memphis pattern, a rectangular grid pattern, a hexagonal pattern, a spiral pattern, a swirl pattern, a radial pattern, an array of lines, a zig-zag or a random pattern, the non-refractive features configured within the optic zone to introduce a luminous contrast profile, i.e. artificial edges, in the retinal imagery. In one embodiment of the present disclosure, the contemplated moiré patterns or moiré fringes may be achieved by producing large-scale interference patterns when an opaque ruled pattern with transparent gaps is overlaid on another similar pattern that is laterally separated. In another embodiment, a moiré pattern may be achieved by printing ruled patterns on both surfaces of the contact lens with a pre-determined offset and orientation. Alternatively, in other embodiments, the resultant moiré pattern may be printed or configured on one surface of the contact lens.

Certain embodiments of the present disclosure are directed to a combination single vision contact lens design, made of a hydrogel material, or a silicon hydrogel material, that incorporates non-refractive features within the optic zone of the single vision contact lens aimed at inhibiting, preventing and/or controlling the progression of myopia.

Some ophthalmic lens embodiments of the disclosure provide a spatiotemporal variation of stop signal facilitated by either the on-eye movement of the ophthalmic lens, for example a contact lens, the natural blink action of the eye-lids while wearing a contact lens of the disclosure, or due to the eye-movements while wearing the contemplated spectacle lens embodiments disclosed herein. The spatiotemporal variation of the presentation of the artificial edge profiles, or luminous contrast profiles, allow for minimisation of the saturation of efficacy with time on the rate of progression of myopia. The embodiments presented in this disclosure are directed to the ongoing need for enhanced ophthalmic lenses that offer a therapeutic benefit of inhibiting, or reducing the rate of progression of myopia, while providing single vision equivalent, or adequate, visual performance to the wearer across a range of distances and viewing angles.

Certain other embodiments of the disclosure are aimed to maintain the potency of the therapeutic benefit over time. Various aspects of the embodiments of the present disclosure address such needs of a wearer. Embodiments of the present disclosure are directed to a contact lens for at least one of slowing, retarding, or preventing myopia progression. The contact lens comprising a front surface, a back surface, an optic zone and an optical centre; wherein the optical zone around the optical centre is configured with a plurality of fine lines, or a plurality of striations, or a plurality of striae and which is otherwise substantially configured with a single vision prescription to provide, at least in part, adequate foveal correction, and further the contemplated design features are configured to provide, at least in part, an increase in retinal ganglion cell activity thus providing a stop signal to reduce the rate of myopia progression.

In accordance with some of the embodiments, a contact lens is configured with a plurality of non-refractive design features, for example, a plurality of lines, or striae, or apertures or patterns, within a substantially single vision optic zone, that provides an active rise in retinal encoding of a spatio-temporal signal facilitated by either the on-eye movement of the contact lens, the natural blink action of the eye-lids or the eye-movements while wearing the contemplated contact lens disclosed herein. Thus, allowing for a minimisation of the saturation of efficacy with time on the rate of myopia progression.

In accordance with some of the embodiments, a spectacle lens is configured with a plurality of non-refractive design features, for example, a plurality of lines, or striae, or apertures or patterns, within a substantially single vision optic zone, that provides an active rise in retinal encoding of a spatio-temporal signal facilitated by the eye-movements while wearing the contemplated spectacle lens disclosed herein. The embodiments presented in this disclosure are directed to the ongoing need for enhanced optical designs of ophthalmic lenses that may inhibit the progression of myopia while providing reasonable and adequate visual performance to the wearer for a range of activities that the wearer may undertake as a part of their daily routine. Various aspects of the embodiments of the present disclosure address such needs of a wearer. An exemplary method of this disclosure includes a measurement of the refractive state of an eye of an individual based on standard optometric refraction techniques; identifying a base prescription for the eye based at least in part on the refraction measurement of the eye, selecting the power of the single vision lens of the disclosure such that it is substantially matched to the base prescription required to correct the underlying refractive error, and further selecting the size, pattern and arrangement of the non-refractive features contemplated in the present disclosure such that the desirable increase in ganglion cell activity at the retina of the individual is balanced with any marginal perception of the visual disturbances that may be experienced by the individual. In one or more embodiments of the present disclosure, the non-refractive features are substantially opaque and positioned within the designated region of the single vision ophthalmic lens; such that these non-refractive features provide an increase in retinal ganglion cell activity in the on- and off-centre retinal pathways disclosed herein. In some methods of this disclosure, the selection of the non-refractive features may depend on the activities that the wearer may undertaking while wearing the ophthalmic device, for example, a wearer that reads and performs activities on a computer or table or phone may be prescribed with a pattern that is different to a wearer who is engaged in distance visual tasks such that the balance between the potency of the therapeutic benefit and visual performance is maintained at desirable levels. In some other methods, the selection of non-refractive features may depend on the underlying risk factors for developing, or experiencing, progressive myopia.

Several other embodiments including the embodiments discussed in the summary are set forth in the description, the drawings, and the claims of the disclosure. Understandably, it is practically not possible to include every single combination of the contemplated embodiments of the disclosure, any combinations, or any variants, which contemplate, at least in part, the underlying concept of increasing retinal ganglion cell activity through the use of non-refractive features in conjunction with ophthalmic lenses is considered to be in the scope of the invention. This summary section of the disclosure is not intended to be limited to the embodiments disclosed herein. Furthermore, any limitations of one embodiment may be combined with any other limitations of any other embodiment to constitute additional embodiments of the present disclosure.

### BRIEF DESCRIPTION OF THE FIGURES

FIG. 1 illustrates the workings of the on-centre/off-surround and the off-centre/on-surround types of retinal receptive fields, according to certain embodiments.

FIG. 2 illustrates the workings of on-centre/off-surround retinal receptive fields, when they are subjected to different stimulus or edge profile conditions, according to certain embodiments.

FIG. 3 illustrates a flowchart outlining the virtual retinal platform used to describe workings of some embodiments of the present disclosure. The virtual retinal platform relies on the three-layer structure of the retina: outer plexiform layer, contrast gain control and ganglion cell layer; these retina-related tools aided in encoding visual scenes into trains of action potentials, as described herein.

FIG. 4 is a basic sample of retinal input images onto the retinal receptors assembled to demonstrate the function of the virtual retinal platform used to describe workings of some embodiments of the present disclosure.

FIG. 5 illustrates the spike train (i.e. raster plot) for a sample neuron position at the retinal receptor plane and average retinal spike rate for one of the basic retinal configurations disclosed herein. Retinal ganglion cell responses to spatially uniform flicker between black dot on white background and white dot on black background.

FIG. 6 illustrates the spike train (i.e. raster plots) for a sample neuron position at the retinal receptor plane and average retinal spike rate for another retinal configuration disclosed herein. Retinal ganglion cell responses to spatially uniform flicker between black dot on white background and white dot on black background.

FIG. 7 illustrates the frontal view and cross-sectional view of an exemplary contact lens embodiment with non-refractive features arranged as multiple circular apertures, not to scale, as disclosed herein.

FIG. 8 illustrates the frontal view and cross-sectional view of another exemplary contact lens embodiment with non-refractive features arranged as multiple hexagonal apertures, not to scale, as disclosed herein.

FIG. 9 illustrates the frontal view and cross-sectional view of yet another exemplary contact lens embodiment with striae as non-refractive features, not to scale, as disclosed herein.

FIG. 10 illustrates the frontal view and cross-sectional view of yet another exemplary contact lens embodiment with grid lines as non-refractive features, not to scale, as disclosed herein.

FIG. 11 illustrates the frontal view of three additional exemplary contact lens embodiments (i.e. moiré pattern, curvilinear pattern, Memphis pattern), not to scale, as disclosed herein. In this figure, only the optic zone portion of the contact lens is illustrated.

FIG. 12 illustrates a schematic diagram of the theoretical retinal ganglion cell activity recorded by the on-centre/off-surround and off-centre/on-surround retinal circuitry, when the incoming light, with a visible wavelength (for example, 555 nm) and a vergence of 0 D, is incident on a −3 D myopic model eye, which is corrected with a single vision contact lens of the prior art.

FIG. 13 illustrates a schematic diagram of the theoretical ganglion cell activity recorded by the on-centre/off-surround and off-centre/on-surround retinal circuitry, when the incoming light, with a visible wavelength (for example, 555 nm) and a vergence of 0 D, is incident on a −3 D myopic model eye, which is corrected with one of the contact lens embodiments disclosed herein.

FIG. 14 represents the source image file of a wide-field visual scene (image of a mobile phone held at near viewing distance) projected onto the retina of a wide-angle schematic eye using non-linear projection routines; wherein the virtual retina is modelled with neuron bundles arranged in a circular pattern.

FIG. 15 represents the source image file of a wide-field visual scene (image of a mobile phone held at intermediate distance) projected onto the retina of a wide-angle schematic eye using non-linear projection routines; wherein the virtual retina is modelled with neuron bundles arranged in a circular pattern.

FIG. 16 represents the source image file of a wide-field visual scene (a Lenna standard image) projected onto the retina of a wide-angle schematic eye using non-linear projection routines; wherein the virtual retina is modelled with neuron bundles arranged in a circular pattern.

FIG. 17 illustrates the frontal view and cross-sectional view of an exemplary contact lens embodiment with non-refractive features as multiple circular shaped apertures in a hexagonal arrangement, not to scale, as disclosed herein.

FIG. 18 illustrates the output spike trains, as obtained from on- and off-cell pathways of the virtual retinal model for the control lens C1, described in Example 1, as disclosed herein. The spike trains (i.e. raster plots) obtained from the on- and off-cells are represented as top and bottom sub-graphs. The Y-axis of the graph represents the discrete neuron bundles, and the X-axis represents time in milli-seconds. The dark portions of the graph represent spikes and white portions represent lack thereof.

FIG. 19 showcases the average spike rate, as a function of time, from the on-cell (top) and off-cell (bottom) pathways of the virtual retinal model, obtained for the control contact lens C1, as described herein in Example 1.

FIG. 20 illustrates the output spike trains obtained from on- and off-cell pathways of the virtual retinal model for the contact lens embodiment D1, described in Example 1, as disclosed herein. The spike trains obtained from the on- and off-cells are represented as top and bottom sub-graphs. The Y-axis of the graph represents the neuron bundle, and the X-axis represents time in milli-seconds. The dark portions of the graph represent spikes and white portions represent lack thereof.

FIG. 21 showcases the average spike rate, as a function of time, from the on-cell (top) and off-cell (bottom) pathways of the virtual retinal model, obtained for the contact lens embodiment D1, as described herein in Example 1.

FIG. 22 showcases the on-axis modulation transfer function of control contact lens C1 and contact lens embodiment D1, evaluated at a 4 mm pupil diameter, as described herein in Example 1.

FIG. 23 showcases the off-axis modulation transfer function of control contact lens C1 and contact lens embodiment D1, evaluated at a field angle of 7.5 degree and a 4 mm pupil diameter, as described herein in Example 1.

FIG. 24 illustrates the frontal view and cross-sectional view of an exemplary contact lens embodiment with dot-like non-refractive features in a hexagonal arrangement pattern, not to scale, as disclosed herein.

FIG. 25 illustrates the output spike trains obtained from on- and off-cell pathways of the virtual retinal model for the control lens C2, described in Example 2, as disclosed herein. The spike trains obtained from the on- and off-cells are represented as top and bottom sub-graphs. The Y-axis of the graph represent the discrete neuron bundles, and the X-axis represents time in milli-seconds. The dark portions of the graph represent spikes and white portions represent lack thereof.

FIG. 26 showcases the average spike rate, as a function of time, from the on-cell (top) and off-cell (bottom) pathways of the virtual retinal model, obtained for the control contact lens C2, as described herein in Example 2.

FIG. 27 illustrates the output spike trains obtained from on- and off-cell pathways of the virtual retinal model for the contact lens embodiment D2, described in Example 2, as disclosed herein. The spike trains obtained from the on- and off-cells are represented as top and bottom sub-graphs. The Y-axis of the graph represents the neuron bundle, and the X-axis represents time in milli-seconds. The dark portions of the graph represent spikes and white portions represent lack thereof.

FIG. 28 showcases the average spike rate, as a function of time, from the on-cell (top) and off-cell (bottom) pathways of the virtual retinal model, obtained for the contact lens embodiment D2, as described herein in Example 2.

FIG. 29 showcases the on-axis modulation transfer function of control contact lens C2 and contact lens embodiment D2, evaluated at a 4 mm pupil diameter, as described herein in Example 2.

FIG. 30 showcases the off-axis modulation transfer function of control contact lens C2 and contact lens embodiment D2, evaluated at a field angle of 7.5 degree and a 4 mm pupil diameter, as described herein in Example 2.

FIG. 31 illustrates the frontal view and cross-sectional view of an exemplary contact lens embodiment with striae as non-refractive features in a random arrangement, not to scale, as disclosed herein.

FIG. 32 illustrates the output spike trains obtained from on- and off-cell pathways of the virtual retinal model for the control lens C3, described in Example 3, as disclosed herein. The spike trains obtained from the on- and off-cells are represented as top and bottom sub-graphs. The Y-axis of the graph represents the discrete neuron bundles, and the X-axis represents time in milli-seconds. The dark portions of the graph represent spikes and white portions represent lack thereof.

FIG. 33 showcases the average spike rate, as a function of time, from the on-cell (top) and off-cell (bottom) pathways of the virtual retinal model, obtained for the control contact lens C3, as described herein in Example 3.

FIG. 34 illustrates the output spike trains obtained from on- and off-cell pathways of the virtual retinal model for the control lens D3, described in Example 3, as disclosed herein. The spike trains obtained from the on- and off-cells are represented as top and bottom sub-graphs. The Y-axis of the graph represents the discrete neuron bundles, and the X-axis represents time in milli-seconds. The dark portions of the graph represent spikes and white portions represent lack thereof.

FIG. 35 showcases the average spike rate, as a function of time, from the on-cell (top) and off-cell (bottom) pathways of the virtual retinal model, obtained for the contact lens embodiment D3, as described herein in Example 3.

FIG. 36 showcases the on-axis modulation transfer function of control contact lens C3 and contact lens embodiment D3, evaluated at a 6 mm pupil diameter, as described herein in Example 3.

FIG. 37 showcases the off-axis modulation transfer function of control contact lens C3 and contact lens embodiment D3, evaluated at a field angle of 2.5 degree and a 6 mm pupil diameter, as described herein in Example 3.

FIG. 38 illustrates the frontal view and cross-sectional view of an exemplary contact lens embodiment with grid lines as non-refractive features, not to scale, as disclosed herein.

FIG. 39 illustrates the output spike trains obtained from on- and off-cell pathways of the virtual retinal model for the control lens C4, described in Example 4, as disclosed herein. The spike trains obtained from the on- and off-cells are represented as top and bottom sub-graphs. The Y-axis of the graph represents the discrete neuron bundles, and the X-axis represents time in milli-seconds. The dark portions of the graph represent spikes and white portions represent lack thereof.

FIG. 40 showcases the average spike rate, as a function of time, from the on-cell (top) and off-cell (bottom) pathways of the virtual retinal model, obtained for the control contact lens C4, as described herein in Example 4.

FIG. 41 illustrates the output spike trains obtained from on- and off-cell pathways of the virtual retinal model for the contact lens embodiment D4, described in Example 4, as disclosed herein. The spike trains obtained from the on- and off-cells are represented as top and bottom sub-graphs. The Y-axis of the graph represents the discrete neuron bundles, and the X-axis represents time in milli-seconds. The dark portions of the graph represent spikes and white portions represent lack thereof.

FIG. 42 showcases the average spike rate, as a function of time, from the on-cell (top) and off-cell (bottom) pathways of the virtual retinal model, obtained for the contact lens embodiment D4, as described herein in Example 4.

FIG. 43 showcases the on-axis modulation transfer function of control contact lens C4 and contact lens embodiment D4, evaluated at a 6 mm pupil diameter, as described herein in Example 4.

FIG. 44 showcases the off-axis modulation transfer function of control contact lens C4 and contact lens embodiment D4, evaluated at a field angle of 7.5 degree and a 6 mm pupil diameter, as described herein in Example 4.

FIG. 45 illustrates the frontal view and cross-sectional view of an exemplary contact lens embodiment with lines, or striae, as non-refractive features, in a radial, or spoke-like, arrangement, not to scale, as disclosed herein.

FIG. 46 illustrates the output spike trains obtained from on- and off-cell pathways of the virtual retinal model for the control lens C5, described in Example 5, as disclosed herein. The spike trains obtained from the on- and off-cells are represented as top and bottom sub-graphs. The Y-axis of the graph represents the discrete neuron bundles, and the X-axis represents time in milli-seconds. The dark portions of the graph represent spikes and white portions represent lack thereof.

FIG. 47 showcases the average spike rate, as a function of time, from the on-cell (top) and off-cell (bottom) pathways of the virtual retinal model, obtained for the control contact lens C5, as described herein in Example 5.

FIG. 48 illustrates the output spike trains obtained from on- and off-cell pathways of the virtual retinal model for the contact lens embodiment D5, described in Example 5, as disclosed herein. The spike trains obtained from the on- and off-cells are represented as top and bottom sub-graphs. The Y-axis of the graph represents the discrete neuron bundles, and the X-axis represents time in milli-seconds. The dark portions of the graph represent spikes and white portions represent lack thereof.

FIG. 49 showcases the average spike rate, as a function of time, from the on-cell (top) and off-cell (bottom) pathways of the virtual retinal model, obtained for the contact lens embodiment D5, as described herein in Example 5.

FIG. 50 showcases the on-axis modulation transfer function of control contact lens C5 and contact lens embodiment D5, evaluated at a 5 mm pupil diameter, as described herein in Example 5.

FIG. 51 showcases the off-axis modulation transfer function of control contact lens C5 and contact lens embodiment D5, evaluated at a field angle of 7.5 degree and a 5 mm pupil diameter, as described herein in Example 5.

FIG. 52 illustrates the frontal view and cross-sectional view of an exemplary contact lens embodiment with dot-like non-refractive features in a random arrangement, not to scale, as disclosed herein.

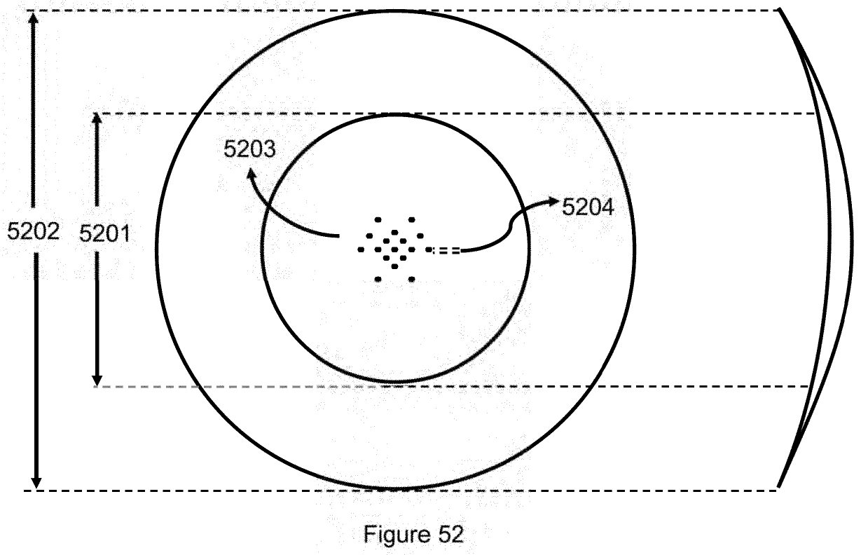

FIG. 53 illustrates the output spike trains obtained from on- and off-cell pathways of the virtual retinal model for the control lens C6, described in Example 6, as disclosed herein. The spike trains obtained from the on- and off-cells are represented as top and bottom sub-graphs. The Y-axis of the graph represents the discrete neuron bundles, and the X-axis represents time in milli-seconds. The dark portions of the graph represent spikes and white portions represent lack thereof.

FIG. 54 showcases the average spike rate, as a function of time, from the on-cell (top) and off-cell (bottom) pathways of the virtual retinal model, obtained for the control contact lens C6, as described herein in Example 6.

FIG. 55 illustrates the output spike trains obtained from on- and off-cell pathways of the virtual retinal model for the contact lens embodiment D6, described in Example 6, as disclosed herein. The spike trains obtained from the on- and off-cells are represented as top and bottom sub-graphs. The Y-axis of the graph represents the discrete neuron bundles, and the X-axis represents time in milli-seconds. The dark portions of the graph represent spikes and white portions represent lack thereof.

FIG. 56 showcases the average spike rate, as a function of time, from the on-cell (top) and off-cell (bottom) pathways of the virtual retinal model, obtained for the contact lens embodiment D6, as described herein in Example 6.

FIG. 57 showcases the on-axis modulation transfer function of control contact lens C6 and contact lens embodiment D6, evaluated at a 4 mm pupil diameter, as described herein in Example 6.

FIG. 58 showcases the off-axis modulation transfer function of control contact lens C6 and contact lens embodiment D6, evaluated at a field angle of 7.5 degree and a 4 mm pupil diameter, as described herein in Example 6.

FIG. 59 illustrates the frontal view and cross-sectional view of an exemplary contact lens embodiment with dot-like non-refractive features in a spiral arrangement, not to scale, as disclosed herein.

FIG. 60 illustrates the output spike trains obtained from on- and off-cell pathways of the virtual retinal model for the control lens C7, described in Example 7, as disclosed herein. The spike trains obtained from the on- and off-cells are represented as top and bottom sub-graphs. The Y-axis of the graph represents the discrete neuron bundles, and the X-axis represents time in milli-seconds. The dark portions of the graph represent spikes and white portions represent lack thereof.

FIG. 61 showcases the average spike rate, as a function of time, from the on-cell (top) and off-cell (bottom) pathways of the virtual retinal model, obtained for the control contact lens C7, as described herein in Example 7.

FIG. 62 illustrates the output spike trains obtained from on- and off-cell pathways of the virtual retinal model for the contact lens embodiment D7, described in Example 7, as disclosed herein. The spike trains obtained from the on- and off-cells are represented as top and bottom sub-graphs. The Y-axis of the graph represents the discrete neuron bundles, and the X-axis represents time in milli-seconds. The dark portions of the graph represent spikes and white portions represent lack thereof.

FIG. 63 showcases the average spike rate, as a function of time, from the on-cell (top) and off-cell (bottom) pathways of the virtual retinal model, obtained for the contact lens embodiment D7, as described herein in Example 7.

FIG. 64 showcases the on-axis modulation transfer function of control contact lens C7 and contact lens embodiment D7, evaluated at a 6 mm pupil diameter, as described herein in Example 7.

FIG. 65 showcases the off-axis modulation transfer function of control contact lens C7 and contact lens embodiment D7, evaluated at a field angle of 7.5 degree and a 6 mm pupil diameter, as described herein in Example 7.

FIG. 66 illustrates the frontal view of an exemplary spectacle lens embodiment with non-refractive features arranged as grid-like pattern disclosed herein, and a spectacle lens of the prior art, not to scale.

FIG. 67 illustrates a schematic diagram of the theoretical retinal ganglion cell activity recorded by the on-centre/off-surround and off-centre/on-surround retinal circuitry, when the incoming light, with a visible wavelength (for example, 555 nm) and a vergence of 0 D, is incident on a −3 D myopic model eye, which is corrected with a single vision contact lens of the prior art.

FIG. 68 illustrates a schematic diagram of the theoretical ganglion cell activity recorded by the on-centre/off-surround and off-centre/on-surround retinal circuitry, when the incoming light, with a visible wavelength (for example, 555 nm) and a vergence of 0 D, is incident on a −3 D myopic model eye, which is corrected with one of the contact lens embodiments disclosed herein.

FIG. 69 illustrates the frontal view of an exemplary spectacle lens embodiment with dot-like non-refractive features in a swirl arrangement with 6 radial arms, not to scale, as disclosed herein.

FIG. 70 illustrates the output spike trains obtained from on- and off-cell pathways of the virtual retinal model for the control spectacle lens C8, described in Example 8, as disclosed herein. The spike trains obtained from the on- and off-cells are represented as top and bottom sub-graphs. The Y-axis of the graph represents the discrete neuron bundles, and the X-axis represents time in milli-seconds. The dark portions of the graph represent spikes and white portions represent lack thereof.

FIG. 71 showcases the average spike rate, as a function of time, from the on-cell (top) and off-cell (bottom) pathways of the virtual retinal model, obtained for the control spectacle lens C8, as described herein in Example 8.

FIG. 72 illustrates the output spike trains obtained from on- and off-cell pathways of the virtual retinal model for the spectacle lens embodiment D8, described in Example 8, as disclosed herein. The spike trains obtained from the on- and off-cells are represented as top and bottom sub-graphs. The Y-axis of the graph represents the discrete neuron bundles, and the X-axis represents time in milli-seconds. The dark portions of the graph represent spikes and white portions represent lack thereof.

FIG. 73 showcases the average spike rate, as a function of time, from the on-cell (top) and off-cell (bottom) pathways of the virtual retinal model, obtained for the spectacle lens embodiment D8, as described herein in Example 8.

FIG. 74 showcases the on-axis modulation transfer function of control spectacle lens C8 and spectacle lens embodiment D8, evaluated at a 6 mm pupil diameter, as described herein in Example 8.

FIG. 75 showcases the off-axis modulation transfer function of control spectacle lens C8 and spectacle lens embodiment D8, evaluated at a field angle of 10 degree and a 6 mm pupil diameter, as described herein in Example 8.

FIG. 76 illustrates the frontal view of an exemplary spectacle lens embodiment with line, or striae, -like non-refractive features, in a grid arrangement, not to scale, as disclosed herein.

FIG. 77 illustrates the output spike trains obtained from on- and off-cell pathways of the virtual retinal model for the control spectacle lens C9, described in Example 9, as disclosed herein. The spike trains obtained from the on- and off-cells are represented as top and bottom sub-graphs. The Y-axis of the graph represents the discrete neuron bundles, and the X-axis represents time in milli-seconds. The dark portions of the graph represent spikes and white portions represent lack thereof.

FIG. 78 showcases the average spike rate, as a function of time, from the on-cell (top) and off-cell (bottom) pathways of the virtual retinal model, obtained for the control spectacle lens C9, as described herein in Example 9.

FIG. 79 illustrates the output spike trains obtained from on- and off-cell pathways of the virtual retinal model for the spectacle lens embodiment D8, described in Example 9, as disclosed herein. The spike trains obtained from the on- and off-cells are represented as top and bottom sub-graphs. The Y-axis of the graph represents the discrete neuron bundles, and the X-axis represents time in milli-seconds. The dark portions of the graph represent spikes and white portions represent lack thereof.

FIG. 80 showcases the average spike rate, as a function of time, from the on-cell (top) and off-cell (bottom) pathways of the virtual retinal model, obtained for the spectacle lens embodiment D9, as described herein in Example 9.

FIG. 81 showcases the on-axis modulation transfer function of control spectacle lens C9 and spectacle lens embodiment D9, evaluated at a 5 mm pupil diameter, as described herein in Example 9.

FIG. 82 showcases the off-axis modulation transfer function of control spectacle lens C9 and spectacle lens embodiment D9, evaluated at a field angle of 10 degree and a 5 mm pupil diameter, as described herein in Example 9.

FIG. 83 illustrates the frontal view of an exemplary spectacle lens embodiment with lines, or striae, -like non-refractive features, in a random arrangement, not to scale, as disclosed herein.

FIG. 84 illustrates the output spike trains obtained from on- and off-cell pathways of the virtual retinal model for the control lens C10, described in Example 10, as disclosed herein. The spike trains obtained from the on- and off-cells are represented as top and bottom sub-graphs. The Y-axis of the graph represents the discrete neuron bundles, and the X-axis represents time in milli-seconds. The dark portions of the graph represent spikes and white portions represent lack thereof.

FIG. 85 showcases the average spike rate, as a function of time, from the on-cell (top) and off-cell (bottom) pathways of the virtual retinal model, obtained for the control spectacle lens C10, as described herein in Example 10.

FIG. 86 illustrates the output spike trains obtained from on- and off-cell pathways of the virtual retinal model for the spectacle lens embodiment D10, described in Example 10, as disclosed herein. The spike trains obtained from the on- and off-cells are represented as top and bottom sub-graphs. The Y-axis of the graph represents the neuron bundle, and the X-axis represents time in milli-seconds. The dark portions of the graph represent spikes and white portions represent lack thereof.

FIG. 87 showcases the average spike rate, as a function of time, from the on-cell (top) and off-cell (bottom) pathways of the virtual retinal model, obtained for the spectacle lens embodiment D10, as described herein in Example 10.

FIG. 88 showcases the on-axis modulation transfer function of control spectacle lens C10 and spectacle lens embodiment D10, evaluated at a 4 mm pupil diameter, as described herein in Example 10.

FIG. 89 showcases the off-axis modulation transfer function of control spectacle lens C10 and spectacle lens embodiment D10, evaluated at a field angle of 10 degree and a 4 mm pupil diameter, as described herein in Example 10.

### DETAILED DESCRIPTION

Optical solutions available for retarding the rate of myopia progression, include some form of optical manipulation of the retinal image characteristics, for example, lenses that utilise simultaneous defocus, positive spherical aberration, positive power in the centre and/or periphery of the optic zone, or higher order aberrations to extend depth of focus.

One of the weaknesses of such optical designs is that they compromise the quality of the vision. Given the influence of compliance of lens wear on the efficacy of such lenses, significant reduction of visual performance may promote poor compliance thus resulting in poorer efficacy.

Accordingly, what is needed are designs for the correction of myopia and retardation of progression, which do not cause the visual disturbances associated with manipulation of optical power within the ophthalmic lens. The current disclosure proposes an alternative non-refractive method to retard the progression of myopia that does not utilise optical defocus as a stop signal. Embodiments of the present disclosure proposes an alternative method to retard the progression of myopia by artificially introducing edges, or luminous contrast profiles to the retinal image. Some embodiments further introduce spatio-temporal variations of the luminous contrast profiles into the imagery that is projected onto the retina through the lenses of the current disclosure, and thereby increasing the overall retinal activity which in turn may inhibit further eye growth. One or more of the embodiments of the present disclosure rely on the centre-surround architecture of the retinal ganglion cells producing preferential responses to spatial and/or temporal changes in the luminous profile incident on the retina.

In this section, the present disclosure is described in detail with reference to one or more contact lens, or one or more spectacle embodiments, some of the contemplated embodiments are illustrated and supported by accompanying figures. Some contact lens and spectacle lens embodiments are provided by way of explanation and are not to be construed as limiting to the scope of the disclosure.

The following description is provided in relation to several contact lens and spectacle lens embodiments that may share common characteristics and features of the disclosure. It is to be understood that one or more features of one embodiment may be combined with one or more features of any other embodiments which may constitute additional embodiments. The functional and structural information disclosed herein is not to be interpreted as limiting in any way and should be construed merely as a representative basis for teaching a person skilled in the art to employ the disclosed embodiments and variations of those embodiments in various ways. The sub-titles and relevant subject headings used in the detailed description section have been included only for the ease of reference of the reader and should in no way be used to limit the subject matter described throughout the disclosure or the claims of the disclosure. The sub-titles and relevant subject headings should not be used in construing the scope of the claims or the claim limitations.

Some of the techniques that have been reported to be usable to identify individuals with a risk of developing myopia or progressive myopia include inquiries on one or more of the following factors: genetics, ethnicity, lifestyle, environmental, excessive near work, etc. Certain embodiments of the present disclosure are directed towards a person identified as at risk of developing myopia or progressive myopia. To date, numerous optical designs have been proposed to control the rate of eye growth or to retard the progression of myopia. Some of these designs are characterised by the use of some degree of relative positive power related to the base prescription. Designs based on such optical principles invite significant compromise of the quality of vision. Given the influence of compliance of lens wear on the efficacy, a significant reduction of visual performance may promote poor compliance thus resulting in poorer efficacy.

Embodiments of the present disclosure relate to optical designs that utilise the effects of purposefully configured non-refractive features within the optic zone of an otherwise single vision ophthalmic lens designed to increase the retinal ganglion cell activity which in turn aids to inhibit or decelerate the rate of myopia progression.

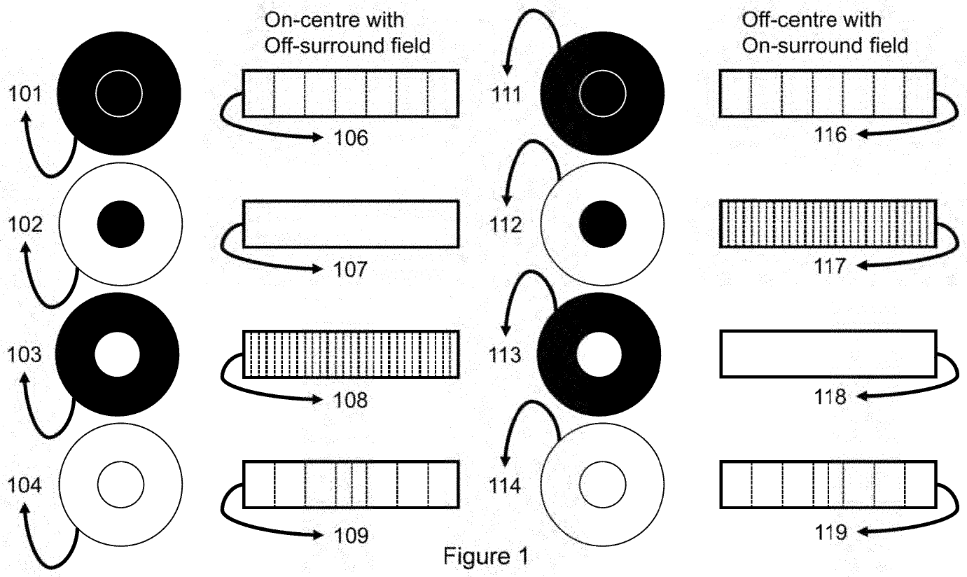

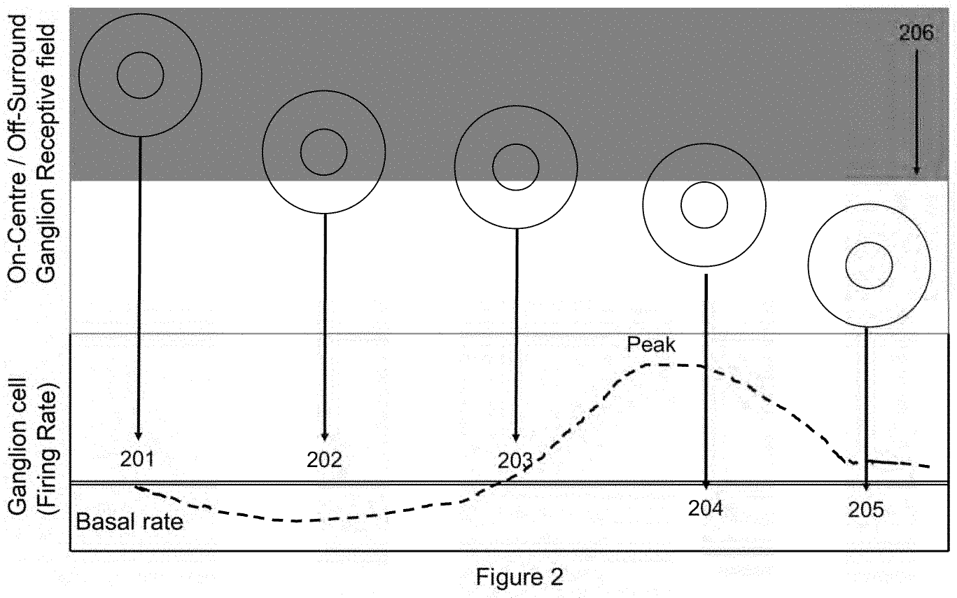

The human visual system is organised into on- and off-retinal channels or pathways. The retinal ganglion cells have circular receptive fields that are organised into on-centre/off-surround bipolar cells or vice versa; whose workings are succinctly described in FIG. 1 and FIG. 2.

The complex retinal ganglion cell circuitry aids in the conversion of the spatiotemporal information contained within the incident light of the visual input scene into spike trains and patterns of activity conveyed to the visual cortex by the axons of the retinal ganglion cells which form the fibres of the optic nerve.

Two groups of retinal ganglion cells, magno and parvo cells, aid in the different types of responses to the incoming light signal captured on the retina. The information carried by magno and parvo cells are parallel and independent to each other.

The magnocellular or transient pathway captures the temporal features of the incoming light signal, for example, motion, changes and onsets within the input scene; while the parvocellular or sustained pathway captures spatial features of the incoming light signal, for example, patterns and shapes within the input scene.

The magnocellular pathway has large receptive fields, short latencies, and responds in a transient way utilising rapidly conducting axons. The parvocellular pathway, on the other hand, has smaller receptive fields, long latencies and responds in a sustained way by utilising slow conducting axons. The relative change events captured by the magnocellular pathway and the grey-level sustained image frames captured by the parvocellular pathway are two highly orthogonal representations of the visual scene.

Given the regulation of eye growth is locally and not globally mediated, the magnocellular pathway may be involved in the regulation of the eye growth, or mediation of the homeostasis of eye growth, for at least some individuals. In other words, the magnocellular retinal ganglion cells, which contain the information on local relative changes, offer the ability to encode the dynamic or temporal contrast within the visual scene which can be transcribed into growth on- or growth off-signals.

Increase in spatiotemporal contrast of a visual scene has a potential to introduce a spike, or a short-duration increase, in the retinal ganglion cell activity; and the greater the retinal ganglion cell activity, the higher the growth inhibition signal for the eye. Due to the construct of the retinal receptive field circuitry, the following two conditions fail to excite the retinal ganglion cells: (a) a homogeneously illuminated retinal scene without distinct edges (i.e. absence of spatial contrast in the visual scenery); or (b) a lack of change in the scenery for too long (i.e. absence of temporal contrast). The lower the excitation of the retinal ganglion cells the lower the firing activity which in turn signifies overall lower retinal activity; and the greater the inactivity of the retina the lower the growth inhibition signal resulting in further eye growth. The relative difference in the temporal integration of on- and off-receptive field activity determines further eye growth.

The present disclosure postulates that an inactive retina triggers eye growth and an active retina inhibits the growth or triggers a stop signal. The present disclosure further contemplates that standard single vision contact lenses or spectacle lenses of the prior art and/or a spatially homogenous visual imagery contributes to a homogeneous and substantially edge-less visual imagery leaving the retina in a baseline state (i.e. baseline or constant firing pattern of the retinal ganglion cells) and thus promoting further eye growth leading to more myopia.

FIG. 1 illustrates the workings of the on-centre with off-surround, and the off-centre with on-surround types of retinal receptive fields, used to describe one or more of the embodiments of the present disclosure.

The first and third columns of FIG. 1 highlight four instances of theoretical stimulus presentation: (a) no light across the retinal receptive field (101 & 111); (b) no light in the central region of the retinal receptive field, while the surround is fully lit (102 & 112); (c) no light in the surround region of the retinal receptive field, while the central region is fully lit (103 & 113); and (d) both the central and surround regions of the retinal receptive field are fully lit (104 & 114). The second and fourth columns showcase the firing action potentials over time for the various corresponding stimulus conditions disclosed in FIGS. 1(a) to 1(d).

For example, when an on-centre with off-surround retinal receptive field is considered (i.e. first two columns of FIG. 1), in the absence of a light stimulus (101), the retinal ganglion cells fire at a baseline rate (106). When the light only falls on the off-surround region (102) and not on the on-centre region, then the baseline firing is suppressed during the stimulus period (107).

When a light spot coincides with the on-centre zone (103), the firing rate of the retinal ganglion cells is at its maximum (108). As the light circle expands to cover both on-centre and off-surround fields (104), the firing pattern diminishes from its maximum and gets closer to the basal firing rate (109).

When an off-centre with on-surround receptive field is considered (i.e. last two columns of FIG. 1), in the absence of a light stimulus (111), the retinal ganglion cells fire at a baseline rate (116).

When the light only falls on the on-surround region (112) and not on the off-centre region then the firing rate of the retinal ganglion cells is at its maximum (117). When a light spot coincides with the off-centre zone (113), the baseline firing is suppressed during the stimulus period (118). As the light circle expands to cover both off-centre and on-surround fields (114), the firing pattern diminishes from its maximum and gets closer to the baseline firing rate (119). It can be appreciated by a person skilled in the art that the illustrations of FIG. 1 are theoretical best-case scenarios that may be difficult to replicate in real life scenarios other than bench-top lab experiments.

FIG. 2 is another graphical illustration of the firing pattern of an on-centre with off-surround retinal receptive field, when subjected to different stimulus conditions. The top half of FIG. 2 showcases five different light stimulus conditions depicting some of the edge (206) detection scenarios that the receptive field may encounter: (i) when the entire receptive field lies in the dark part of the edge (201); (ii) when a part of the surround is in the bright side of the edge, while the centre and the remainder of the off-surround zone are still in the dark part of the edge (202); (iii) when a part of the off-surround and the on-centre region is on the bright side of the edge, while the majority of the on-centre and off-surround region is in the dark patch of the edge (203); (iv) when all of the on-centre region is in the bright side of the edge, while some of the off-surround region is on the dark side of the edge (204); and lastly (v) when the entire receptive field is in the bright side of the edge (205).

The bottom half of FIG. 2 showcases the ganglion cell firing action potential for the five different edge detection scenarios (201-205) that the receptive field may encounter over time. For example, when the entire receptive field lies in the dark part of the edge (201), the firing rate of the ganglion cells is at a basal rate, shown as the double black solid line of FIG. 2. When a part of the off-surround region is in the bright side of the edge, while the on-centre is still in the dark side of the edge (202), the firing rate of the ganglion cells is suppressed below the basal rate. When a part of the off-surround and the on-centre region moves towards the bright side of the edge (203), the firing rate is back to the basal rate. When the entire central region is in the bright side of the edge with some of the surround on the dark side (204), the firing rate achieves its peak.

Finally, when the entire receptive field is in the bright side of the edge (205), the firing rate drops close towards the basal rate but slightly on the higher range. The surround of the receptive field also influences the amount of glutamate released by the photoreceptors. If the surround field is dark, then the photoreceptors in this region will depolarise causing release of more glutamate.

When light falls on the on-centre region, while at least some portion of the off-surround experiences relative darkness, the horizontal cells connected to the photoreceptors in the surround field will depolarise in response to the glutamate and release their own inhibitory neurotransmitter, which will further inhibit the centre photoreceptors to make them release even less glutamate. This situation will create the highest response in the firing action potential of the retinal ganglion cells. Exactly the opposite happens when the surround is in presence of light. The photoreceptor will hyperpolarise in the surround causing it to release less glutamate.

The horizontal cells connected to the photoreceptors in the surround field will hyperpolarise in response and release less of their own inhibitory neurotransmitters, which produces less inhibitory response allowing the central photoreceptor not to be inhibited and release even more glutamate. This is a situation that will create the highest response in an off-centre ganglion receptive field.

### Virtual Retinal Models

It can be appreciated by a person skilled in the art that the illustrations of FIG. 2 are theoretical scenarios of the working models of diverse on- and off-channel retinal fields and they may not be reflective of typical real-life scenarios experienced by individual eyes. To show pertinence to various real-life test cases, a virtual retinal simulation platform is utilised to demonstrate the workings of various embodiments. The operating principles and technical framework of the virtual retinal platform utilised is described herein.

The virtual retinal platform is configured to utilise a set of retinal images, comprising a temporal sequence, as an input and convert them into an output of a set of spike trains or action potentials, which signify the overall activity of the retina. In essence, the edge-detection ability of the centre-surround architecture of the ganglion cells offering a preferential response to spatial and/or temporal changes to the incoming visual scenes was utilised herein. Several variables within the framework of the virtual retinal platform can be tailored to finetune the emulation of wide-field retinal images to mimic real-life scenarios.

Some information about the retinal circuitry and neurophysiology described in the following scientific articles is required to perform the invention disclosed herein. Herewith, a scientific journal article entitled “Probing the potency of Artificial Dynamic On- or Off-stimuli to inhibit myopia development” written by Wang, Aleman and Schaeffel and published in Investigative Ophthalmology and Vision Science journal in June 2019 is referenced herein in its entirety. Another article written by Wohrer and Kornprobst and published in the Journal of Computational Neuroscience in 2009 entitled “Virtual Retina: A biological retina model and simulator, with contrast gain control” is referenced herein in its entirety. In addition, another scientific article entitled “A New Platform for Retinal Analysis and Simulation” written by the authors Cessac, Kornprobst, Kraria, Nasser, Pamplona, Portelli, and Viéville, and published in the journal of Frontiers of Neuroinformatics in 2017, is also referenced herein in its entirety.

Ideally, the source input retinal images for the virtual retinal platform should be a close representative of the images formed on the individual human retina, obtained when an individual is wearing one of the contemplated embodiments disclosed herein. As the actual retinal images are not accessible, the workings of the contemplated images can be emulated using schematic model eyes fitted with the disclosed embodiments, or alternatively, the images can be obtained using physical model eyes fitted with embodiments disclosed herein.

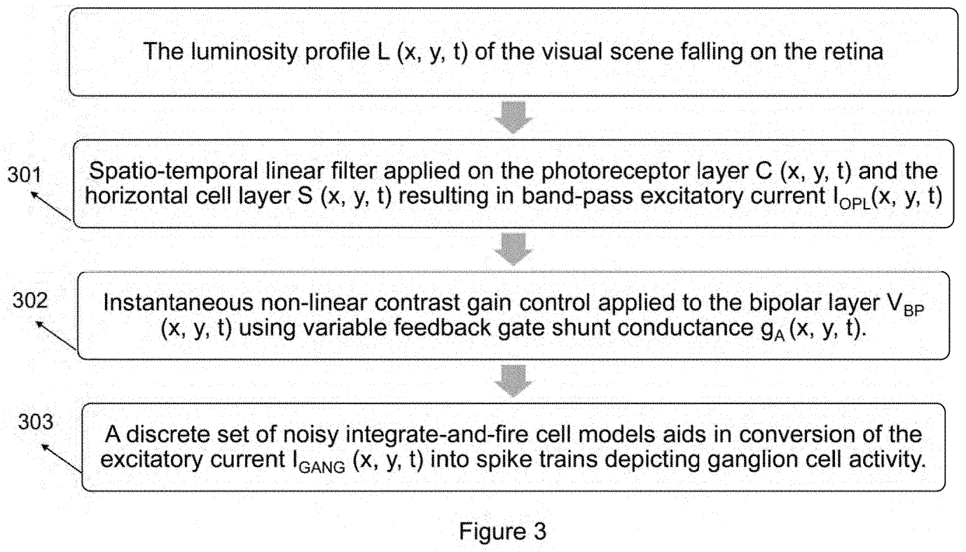

The current disclosure extensively utilises advanced ray-tracing and schematic modelling to obtain virtual retinal images of various objects, when a range of ametropic schematic model eyes are fitted with a range of embodiments disclosed herein. For other embodiments, one may consider alternative approaches which involve utility of a physical or bench-top model eye to demonstrate the workings of the disclosed embodiments. Established models of virtual retinal processing were utilised to describe the workings of various ophthalmic lens embodiments of the present disclosure. FIG. 3 represents the flow chart of the global structure of the virtual retinal model utilised as a platform to describe the inner workings of various embodiments disclosed herein. This model is adapted from the work of Wohrer and Kornprobst published as a peer review paper entitled “Virtual Retina: A biological retina model and simulator, with contrast gain control”.

The proposed three-layer architecture (FIG. 3) of a virtual retinal model facilitates successive continuous spatiotemporal maps that progressively transmit and transform the incoming signal present in a visual scene. The incoming retinal signal has a luminosity profile of L (x, y, t); wherein the luminance is defined for every spatially separated point or pixel (x, y) of the retina at a time point (t). For all simulations used to describe the embodiments of the present disclosure, the input visual scenes were digitised to have intensities between 0 and 255 representing an 8-bit grey level. However, use of input images with intensities between 0 and 1023, or 0 and 4095, or 0 and 65535, representing a 10-bit, or a 12-bit, or a 16-bit grey levels may also be used to demonstrate the utility of other embodiments of the present disclosure. The subsequent layers of the virtual retinal cells are modelled as spatial continuums driven by a set of mathematical equations described herein.

As noted from the chart of FIG. 3, the first stage of virtual retinal model involves processing of the input signal in the outer plexiform layer which involves photoreceptors and horizontal cells. In the first stage, a simple spatiotemporal linear filter based on the teachings of Wohrer and Kornprobst referenced herein is used to decompose the input sequence L (x, y, t) into the photoreceptor centre response C (x, y, t) and response of the horizontal surround cells S (x, y, t). Further, the responses C (x, y, t) and S (x, y, t) are utilised in the outer plexiform layer filter to define a band-pass excitatory current I_{OPL} (x, y, t) which is then fed to the bipolar cells in the second stage of the model. An instantaneous non-linear contrast gain control is applied to the bipolar layer V_{BP} (x, y, t) using variable feedback gate shunt conductance g_{A} (x, y, t) resulting in the excitatory current I_{GANG} (x, y, t). In the third stage, a discrete set of equations governing the noisy integrate-and-fire cell models aids in the conversion of I_{GANG} (X, y, t) into spike trains used to gauge the retinal ganglion cell activity. The spikes can be modelled using one-to-one connections or alternatively using synaptic pooling of the excitatory current received.

To approximate the signal transformations that occur in the layers of the retina, multiple linear filters are used in different stages of the model. To simplify the complexity of the computations and to minimise large computational inefficiencies, while maintaining pertinence to the real-world, some assumptions are made in the model to describe the workings of the embodiments of the present disclosure.

The present disclosure is not limited to the virtual retinal models to describe the workings of the embodiments and use of modifications to the disclosed models and alterative models for design or verification are considered to be within the scope of the invention. In the first stage of the virtual retinal model occurring in the outer plexiform layer, the resulting current I_{OPL}(x, y, t) received by the bipolar cells from the photoreceptors C (x, y, t) and horizontal cells S (x, y, t) is obtained as:

I_{OPL}(x,y,t)=λ_{OPL}(C(x,y,t)−w_{OPL}S(x,y,t)) Equation 1:

C(x,y,t)=G_{σC}(_{*}^{x,y})T_{ωU,τU}(_{*}^{t})E_{ηC,τC}(_{*}^{t})L(x,y,t) Equation 2:

S(x,y,t)=G_{σS}(_{*}^{x,y})E_{τS}(_{*}^{t})C(x,y,t) Equation 3:

In Equation 1, C (x, y, t) represents the centre signal associated with photoreceptors; and S (x, y, t) represents the surround signal associated with horizontal cells. The phototransduction process is modelled as a partially transient linear kernel cascade with exponential temporal low-pass kernel E_{τS} and Gamma exponential cascade E_{ηC,τC} modulated by a partially transient filter τ_{ωU,τU}.

The symbol C in Equation 2 represents a kernel operation on the centre signal, U stands for undershoot, and S in Equation 3 represents a kernel operation on the surround signal. The function G_{σC} in Equation 2 encompasses the spatial blur of the gap junctions between the photoreceptors.

The function G_{σS} in Equation 3 encompasses the spatial blur of the coupling gap junctions between the horizontal cells. The sign (_{*}^{t}) in Equations 2 and 3 denotes temporal convolution; while the sign (_{*}^{x,y}) denotes spatial convolution. signs are used henceforth in this disclosure to denote temporal and spatial convolution. The constant λ_{OPL} is the overall gain of the centre-surround filter; while w_{OPL} is the relative weight of the centre and surround signals.

The contrast gain control operation in the second stage of the virtual retinal model describes the influence of the local contrast of the visual input scene on the electrical signal transfer properties of the retina, which is intrinsically nonlinear and dynamic. The contrast gain control based on a nonlinear feedback loop at the level of bipolar cells can be described as:

$$\frac{d}{dt} ( {V}_{B P} ) ( x , y , t ) = {I}_{O P L} ( x , y , t ) - {g}_{A} ( x , y , t ) * ( {V}_{B P} ) ( x , y , t ) \qquad Equation 4$$

$${g}_{A} ( x , y , t ) = {G}_{σ A} ( \begin{matrix}x , y \\ *\end{matrix} ) {E}_{τ A} ( \begin{matrix}t \\ *\end{matrix} ) Q ( {V}_{B P} ) ( x , y , t ) \qquad Equation 5$$

$$Q ( {V}_{B P} ) = {g}_{A}^{0} + {λ}_{O P L} {V}_{B P}^{2} \qquad Equation 6$$

In Equations 4, 5 and 6, g_{A} represents a variable leakage in the membranes of the bipolar cells which can be activated using a static function QV_{BP}. The leakage determines the gain of the current integration at this level with a divisive effect of g_{A} on the evolution of V_{BP}. In the models, g_{A} depends dynamically on the values considered by bipolar cells with a time scale τA and spatial extent σA.

The third stage of the virtual retinal model involves the generation of spike trains of the retinal ganglion cells from the bipolar cells' activities. The bipolar signal V_{BP} is rectified and receives additional spatiotemporal shaping to produce an excitatory current on ganglion cells I_{GANG} (x, y, t), described in Equations 7 and 8.

$${I}_{G A N G} ( x , y , t ) = {G}_{σ G} ( \begin{matrix}x , y \\ *\end{matrix} ) N [ ε {T}_{ω G , τ G} ( \begin{matrix}t \\ *\end{matrix} ) ( {V}_{B P} ) ( x , y , t ) ] \qquad Equation 7$$

$$N ( V ) = \{ \begin{matrix}\frac{{i}_{G}^{0}}{1 - {λ}_{G} ( V - {v}_{G}^{0} ) / {i}_{G}^{0}} if V < {v}_{G}^{0} \\ {i}_{G}^{0} + {λ}_{G} ( V - {v}_{G}^{0} ) if V \geq {v}_{G}^{0}\end{matrix} \qquad Equation 8$$

The model proposed by Wohrer and Kornprobst used an empirical formula to model signal shaping in the transition from bipolar cells to centre-surround ganglion cell current. These models were adapted for demonstrating the workings of one or more of the embodiments disclosed herein.

The model proposes use of a number of variables allowing for diversity in the functional reproduction of the responses expected from an alternate biologically plausible model, as described in Equations 7 and 8. The parameter c takes two input values −1 and +1, wherein a negative value represents the off-ganglion cell activity and a positive value allows for representation of on-ganglion cell activity.

The bipolar layer signal is rectified using static nonlinear function N(V); wherein the parameters λ_{G} and i_{G}⁰ have the dimensions of reduced currents; while v_{G}⁰ is the linearity threshold of the ganglion cells. Some additional models were proposed by Masmoudi, Antonini and Kornprobst, in a paper entitled “Streaming an image through the eye: the retina seen as a dithered scalable image coder” published in journal of signal processing: Image Communication, Volume 28 (2013), which is incorporated herein in its entirety. From I_{GANG} (x, y, t), an array of noisy leaky-integrate-and-fire neurons (nLIF), produces the set of output spikes. In real retinas, additional complex transformations of the electrical signal occur, facilitated by the synaptic structures of the inner plexiform layer, which is the locus of synaptic interactions between bipolar cells, amacrine cells and ganglion cells.

For the purpose of the modelling to demonstrate the effects of embodiments of the disclosure, the complex synaptic relationships between the amacrine cells, and bipolar cells are ignored in lieu of computational efficiency in some examples.

While in some other examples, one or more of the complexities of the interactions between horizontal cells and bipolar cells, amacrine cells and bipolar cells are taken into consideration, as disclosed herein. Further extension of the model to include various other plausible combinations of outer and inner plexiform layer interactions to describe the workings of contemplated ophthalmic lens embodiments of the disclosure are considered within the scope of the invention.

The transformation of the continuous signal I_{GANG} (x, y, t) into a discrete set of spike trains are obtained from the output of cells using a standard nLIF model described as:

$$\frac{d}{dt} ( {V}_{n} ) ( t ) = {I}_{G A N G} ( {x}_{n} , {y}_{n} , t ) - {g}^{L} * ( {V}_{n} ) ( t ) + ( {η}_{υ} ) ( t ) \qquad Equation 9$$

The standard nLIF model spikes when threshold is reached: (Vₙ)(t)=1 and in the refractory period: (Vₙ)(t)=0. Wherein (ηᵥ)(t) is a noise source that can be added to the spike generation process in order to reproduce the variability in the real ganglion cells.

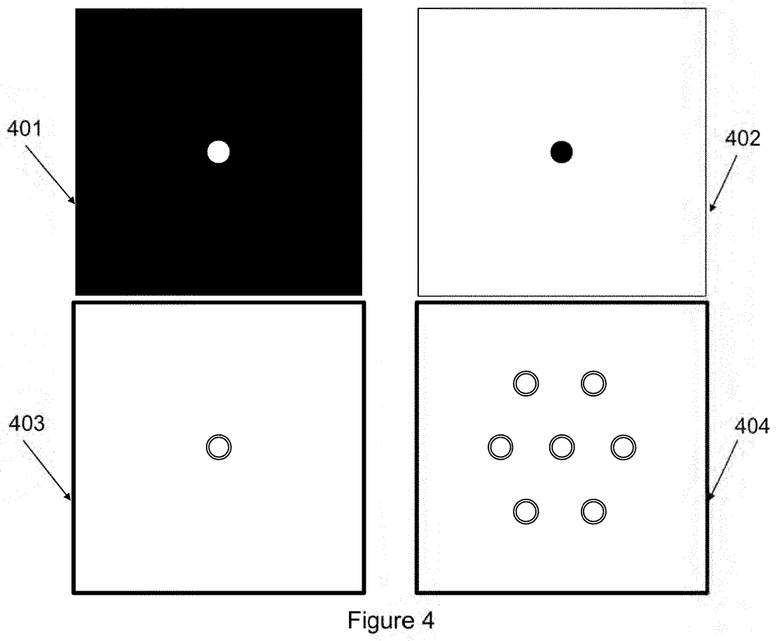

To emulate the spikes of the retinal ganglion cell layer, a virtual retina was defined in the model using the following parameters that offer relative biological plausibility and adaptable degree of complexity. The following example of FIG. 4 establishes the validity of the virtual retinal model described in the paragraphs [00179] to [00200] of the disclosure, configured with certain specific retinal parameters described herein.

In this example, a series of 50 image frames, each with a dimension of 512×512 pixels, were configured as an image montage to serve as an input source for the virtual retinal model. The odd numbered frames of the video input stream consisted of a central circular bright region on a dark background (401), while the even numbered frames were configured with a central circular dark region on a white background (402).

In this example, each frame was configured to be presented for 50 milliseconds accounting for 2.5 seconds of real time stimulation presentation for the virtual retinal model. For both the odd and even frames of the video input stream, the diameter of the central circular region was configured to be approximately 50 pixels, which is equivalent of 0.5° angular subtense of the fovea. The bit depth for each pixel in the input stream were digitised to range from 0 to 255 (i.e. 8-bit). The angular subtense of the video input stream was configured such that each frame subtended approximately 5°×5° on the foveal region of the model retina.

Two simulation test conditions were used to calculate the retinal ganglion cell activity when the input image streams were presented on the virtual retina. The simulations were run in two different cell polarities: on- and off-cell modes. The retinal activity was gauged by the spike activity emanating from the ganglion cell layer of the virtual retinal model. The spike activity for each test condition was represented as average neuronal spike train for each bundle and as a pen-stimulus histogram representation showcasing the average spike rate as a function of time.

The first test condition included one neuron bundle (403), which was positioned such that the centre of video input stream coincided with the centre of the circular neuron bundle. The second test condition included seven circular neuron bundles (404), which were position in a hexagonal pattern with one bundle at the centre of the video input stream and the remaining six bundles arranged circumferentially such that the circumference diameter subtended approximately 2.5°×2.5° on the foveal region of the model retina.

Additionally, for demonstrating the workings for the virtual retinal platform, in this example, the outer plexiform layer was configured to have a centre region subtending approximately 1.5° (i.e. σC of Equation 2) and a surround region subtending approximately 4.75° (i.e. σS of Equation 3). The centre and surround temporal scale of the outer plexiform layer were set to approximately 1 milli-seconds, which represent variables τC and τS of Equations 2 and 3, respectively. The variables governing the integration centre-surround signals, as described in the Equation 1 herein, were chosen to be w_{OPL}=1 and λ_{OPL}=10.

Given the simplicity of the input image stimulus characteristics considered in this example of FIG. 4, the option for contrast gain control mechanism and lateral connectivity of the amacrine cells were muted when computing the spike train and spike rate analysis. The static nonlinearity coefficients of the bipolar and ganglion cell synapses were adapted from Wohrer and Kornprobst, wherein the bipolar linear threshold was set to 0, while the linear threshold value was held constant at 80, and the bipolar amplification value at 100.

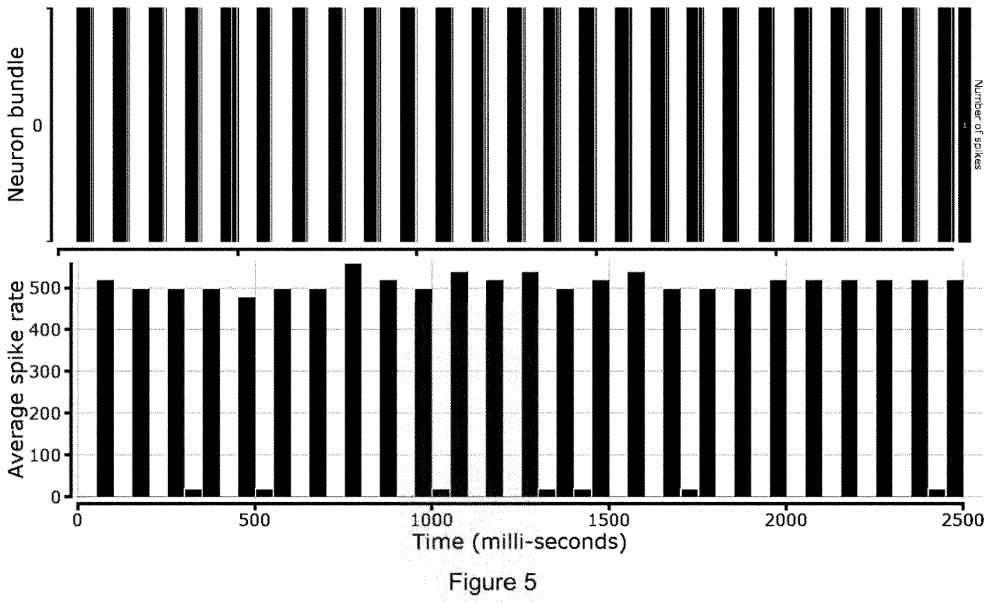

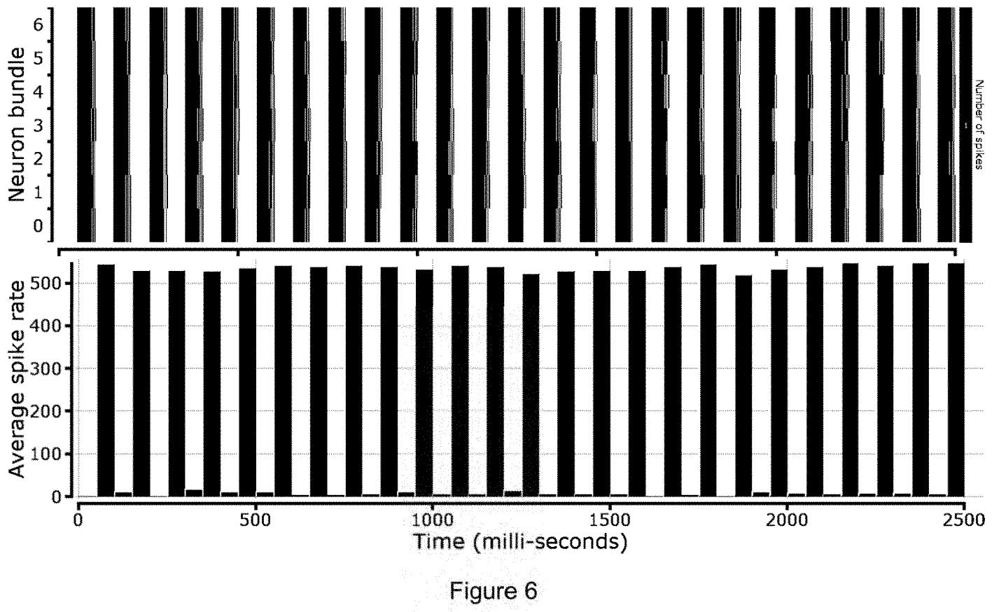

The values for neuron model were also adapted from Wohrer and Kornprobst, wherein a leak of 0.75, neuronal noise of 20, membrane capacitance of 150 and firing threshold of 2.4 were considered for the example described in FIGS. 4, 5 and 6. The post-synaptic pooling variable Sigma was ignored.

To demonstrate the workings of one or more ophthalmic lens embodiments of the present disclosure, static nonlinearity coefficients of the bipolar and ganglion cell synapses may be different from those used for the example of FIG. 4. For example, in some embodiments the bipolar linear threshold may be at least 2, at least 5, at least 10 or at least 15.

To demonstrate the workings of one or more ophthalmic lens embodiments of the present disclosure, the linear threshold value may be a constant value of at least 30, at least 60, at least 90 or at least 120. To demonstrate the workings of one or more ophthalmic lens embodiments of the present disclosure, the bipolar amplification value may be at least 50, at least 75, at least 125 or at least 150.

To demonstrate the workings of one or more ophthalmic lens embodiments of the present disclosure, the leak of neuron model may be set to a value of at least 0.25, at least 0.5, at least 1 or at least 1.25. To demonstrate the workings of one or more ophthalmic lens embodiments of the present disclosure, the neuronal noise may be set to at least 10, at least 25 or at least 50.

To demonstrate the workings of one or more ophthalmic lens embodiments of the present disclosure, the firing threshold of the neurons may be set to at least 1.2, at least 2.4 or at least 3.6.

In various other example embodiments used to describe the workings for the contact lens and spectacle lens embodiments of the present disclosure, various configurations may be contemplated with varying degrees of complexities, as described in Equations 1 to 9, as described herein. The specific configuration settings used for each of the simulations of contact lens embodiments of Examples 1 to 7 are described in the below sections.

### Non-Refractive Features of Disclosed Embodiments

Due to the arrangement of the retinal pathways into the on- and off-channels, in the temporal domain, the retinal neurons respond predominantly to rapidly increasing luminance (on-cells) or decreasing luminance (off-cells) within a visual scene. In the spatial domain, the retinal receptive fields are arranged in a circular pattern into a centre-on and surround-off region or vice versa. Such an arrangement of the retinal cells allows for optimised utilisation of the retinal circuitry to achieve the desired visual processing while maintaining adequate spatial and/or temporal resolution.

A definitive lack of spatial and/or temporal variation within the visual scenes captured at the retinal plane results in poor excitation of retinal ganglion cells and poor retinal activity, or an inactive retina, or insufficiently active retina, is postulated to trigger eye growth. Certain embodiments of the present disclosure are directed towards a person at a risk of developing myopia or progressive myopia. One or more of the embodiments of the present disclosure rely on the hypothesis that a definitive lack of distinct edges, temporally varying distinct edges, or spatial luminous contrast profiles, or temporally varying spatial luminous contrast profiles, across the retina, may contribute towards retinal ganglion cell activity that is akin to its baseline state, in other words a substantially inactive retina.

The output of all receptive fields is integrated, reflecting the relative on- and off-input strengths for a visual environment. The relative difference in the temporal integration of on- and off-receptive field activity is postulated to determine further eye growth. The present disclosure postulates that an inactive retina triggers eye growth and an active retina inhibits the growth or triggers a stop signal.

The present disclosure further contemplates that standard single vision ophthalmic lenses of the prior art and/or a spatially homogenous visual imagery contributes to a homogeneous and substantially spatially edge-less visual imagery leaving the retina in a baseline state (i.e. baseline or constant firing pattern of the retinal ganglion cells) and thus promoting further eye growth leading to more myopia.

One or more of the following advantages are found in one or more of the disclosed optical devices, and/or methods of ophthalmic lens designs disclosed herein. An ophthalmic lens or method providing a stop-signal to retard the rate of eye growth or stop the rate of eye growth, or increase in the state of refractive error, of the wearer's eye based on an increase in retinal activity by employing a plurality of nonrefractive features and artificially introducing edges or enhanced luminous spatial contrast profiles or enhanced temporal contrast profiles into the retinal imagery produced through configuration of contemplated design features on the ophthalmic lens.

The on-eye movement of the contact lens can further augment the strength of the treatment effect by providing a spatially and temporally varying stop signal for increasing the effectivity of managing progressive myopia.

Certain other embodiments are directed to a contact lens device or method that is not solely based on optical manipulation of defocus, astigmatism, or positive spherical aberration, all of which may suffer from the potential visual performance degradation for the wearer. The following exemplary embodiment is directed to methods of modifying the incoming light through an ophthalmic lens that can utilise the selective effects of on and off-visual pathways on eye growth and myopia progression.

The following exemplary embodiment is directed towards methods of modifying the incoming light through an ophthalmic lens that offers increased retinal ganglion activity by stimulation of the on-pathways on the retina by artificially introducing inhomogeneity into the visual imagery and by creating, or increasing, the luminous contrast profiles (i.e. artificial edges) at the retinal plane of the corrected eye. This may be achieved by using substantially opaque borders of a plurality of apertures, zones, segments, or regions within an otherwise single vision optical zone of the ophthalmic lens.

In short, the use of the contemplated multiple apertures, non-refractive regions or non-refractive zones within the optical zone of an otherwise single vision contact lens or spectacle lens, may provide an increase of the activity of the retinal ganglion cells by stimulating the on- and/or off-pathways that are excited by the artificially introduced spatial edge profiles, when the light passes through the contact lens or spectacle lens.

Further, this use of excitatory zones, non-refractive regions, or a plurality of apertures within a single vision contact lens or spectacle lens may offer a variation in temporal contrast supplemented by eye movements and/or blink action of the lids using contact lens and spectacle lens embodiments disclosed herein.

### Schematic Eyes & Simulated Retinal Images

Advanced schematic model eyes may be used for computing wide-field simulated retinal images and wide-field optical performance of one or more of the exemplary embodiments disclosed herein.

The generic prescription of the schematic model eye used for obtaining the retinal images serving as input to the virtual retinal platform used to simulate the workings for embodiments of the present disclosure is provided in the following Table 1. The described parameters in Table 1 are not necessary to demonstrate the described effects obtained with the embodiments of the present disclosure. This should be considered as one of many methods of obtaining retinal images to facilitate the emulation of the retinal processing performed by the virtual retina platform described herein. For example, in other exemplary embodiments, other model eyes of the literature may be used instead of the model eye described in Table 1. The generic parameters of the schematic model eyes used are based on the prescription tabulated in the Table 1. In this example, the generic prescription of Table 1 offers the schematic model eye with a distance refractive error with 1 D of myopia without any astigmatism (Rx: −1 D), configured in its unaccommodated state, wherein the distance prescription of the model eye was defined at a 6 mm pupil diameter and a primary wavelength of 589 nm.

TABLE 1
Prescription of a schematic myopic model eye with a distance
refraction prescription of −1 D.

| | | | Thick- | | Semi | |
|---|---|---|---|---|---|---|
| Surface | | Radius | ness | Refractive- | Diameter | Conic |
| Type | Notes | (mm) | (mm) | Index | (mm) | Const |
| Standard | | Infinity | Infinity | | 0 | 0 |
| Standard | Start | Infinity | 5 | | 4 | 0 |
| Biconic | Anterior | 7.75 | 0.55 | 1.376 | 5.75 | −0.25 |
| | Cornea Y | 7.75 | | | | −0.25 |
| | Anterior | | | | | |
| | Comnea X | | | | | |
| Standard | Posterior | 6.4 | 3 | 1.334 | 5.5 | −0.4 |
| | Cornea | | | | | |
| Standard | Pupil | Infinity | 0.45 | 1.334 | 5 | 0 |
| Standard | Anterior | 10.8 | 3.8 | 1.423 | 4.5 | −3.139 |
| | Lens | | | | | |
| Standard | Posterior | −6.25 | 16.85 | 1.334 | 4.5 | −4.101 |
| | Lens | | | | | |
| Standard | Retina | −12 | 0 | | 10 | 0 |

In the various other example embodiments disclosed herein, various modifications may be considered to evaluate the performance of other ophthalmic lens embodiments described herein. Furthermore, the individual parameters of the schematic model eyes, for example, anterior cornea, posterior cornea, corneal thickness, anterior lens, posterior lens, lens thickness, refractive index of the ocular media, retinal curvature, or combination thereof, may be altered to demonstrate the workings of the present disclosure in various levels of myopia with or without astigmatism, and for the modelling of various myopic eyes in their relaxed and accommodative states.

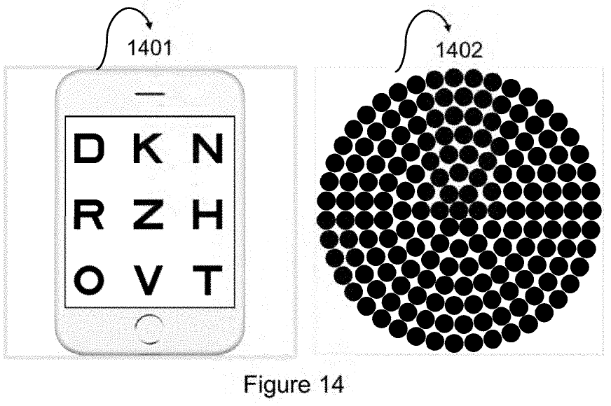

To obtain the wide-field simulated retinal images using the schematic model eyes when fitted with various embodiments of the present disclosure, the source image files were convolved with an array of point spread functions spanning a desired field of view, considering the non-linear projection of the visual scene into a wide-angle schematic eye, as disclosed herein. The three source image files used in one or more of the embodiments are showcased in FIGS. 14, 15 and 16. The first source image showcased on the left portion of the FIG. 14 is a source image file of a mobile phone screen display against a white background screen, wherein the mobile phone screen display is configured with some legible characters and the angular subtense of the source scene was configured to capture a 15 degree visual field at a 50 cm viewing distance.

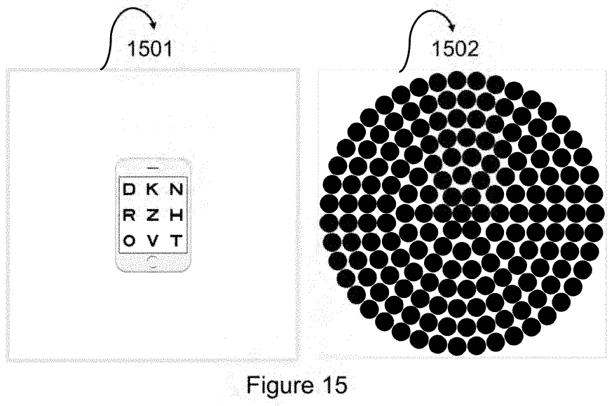

FIG. 14 represents the source image file of a wide-field visual scene (1401) projected onto the retina of a wide-angle schematic eye using non-linear projection routines; wherein the virtual retina is modelled with neuron bundles arranged in a circular pattern (1402). The wide-field visual scene of a mobile phone against a white background (1401) and the frame representing the virtual retina (1402) both subtend approximately 5, 15 or 20° of the retinal field in various embodiments. The second source image illustrated on the left portion of the FIG. 15 is a source image file of another mobile phone screen display against a white background screen, wherein the mobile phone screen display is configured with some legible characters and the angular subtense of the source scene was configured to capture 15° of the visual field at a 1 meter viewing distance.

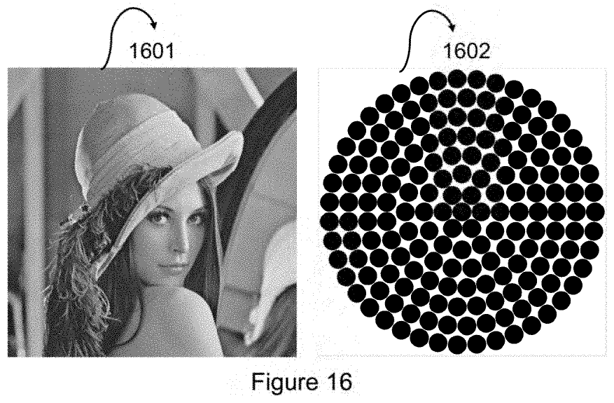

FIG. 15 represents the source image file of a wide-field visual scene (1501) projected onto the retina of a wide-angle schematic eye using non-linear projection routines; wherein the virtual retina is modelled with neuron bundles arranged in a circular pattern (1502). The wide-field visual scene of a mobile phone against a white background (1501) and the frame representing the virtual retina (1502) both subtend approximately 5, 15 or 20° of the retinal field in various embodiments. The third source image file showcased on the left portion of the FIG. 16 is a source image file of a 8-bit grayscale Lenna image; wherein the Lenna image could be configured into two variations, subtending either a 5 degree or a 15 degree or a 20 degree visual field at 6 meters viewing distance.

FIG. 16 represents the source image file of a wide-field visual scene (1601) projected onto the retina of a wide-angle schematic eye using non-linear projection routines; wherein the virtual retina is modelled with neuron bundles arranged in a circular pattern (1602). The wide-field visual scene of the standard Lenna test image presented in 8-bit grayscale (1601) and the frame representing the virtual retina (1602) both subtend approximately 5, 15 or 20° of the retinal field in various embodiments.

The array of point spread function is interpolated for every pixel in the modified image file. At each pixel, the effective point spread function was convolved with the modified source image file.

To compute the point spread function at the desired field, Huygens's principle was adapted in the present disclosure, as the modelled effects of the relatively small non-refractive features may be compromised by the Fourier estimations that are often used for increased computational efficiency.

The computation of the array of point spread functions across the desired field of view includes the effects of diffraction and aberrations. The resulting simulated retinal image is scaled and stretched to account for the detected distortion levels. The brightness of the simulated retinal image is determined by normalizing the intermediate output image to have the same peak brightness as the input source image considered for the convolution operation disclosed herein.

In various embodiments of the present disclosure, the settings of various parameters needed for simulation of virtual retinal images were altered to capture various real-life scenarios that may be experienced by an individual.

In certain embodiments, as the accuracy of the retinal image simulation is limited by the resolution of the input source image, due care was borne to at least maintain a input image resolution of 512×512 pixels to avoid apparent pixel discretisation of the output image often manifested by aliasing effects, and further, wherever necessary oversampling of the input source was considered to minimise such effects at the expense of relatively longer computation time.

### Contact Lens Embodiments

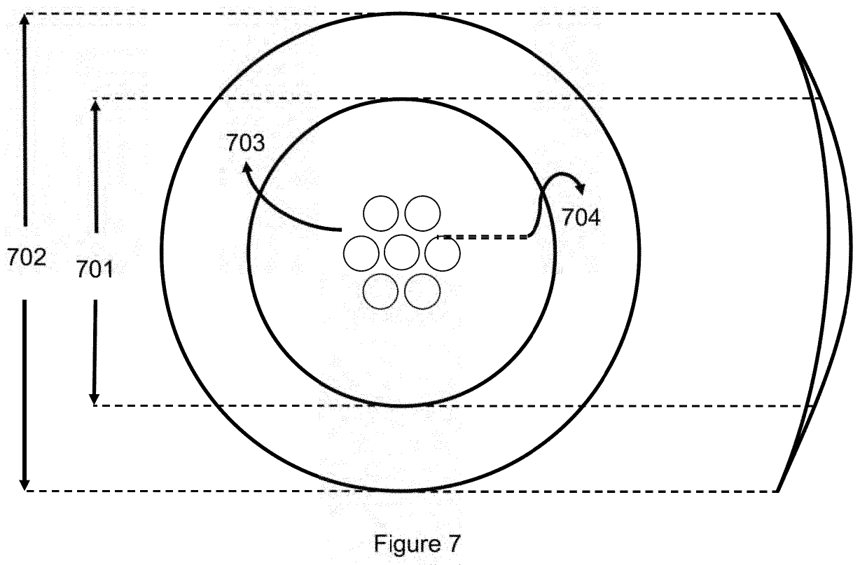

FIG. 7 shows the frontal and cross-sectional view of an exemplary contact lens embodiment, not to scale. The frontal view of the exemplary contact lens embodiment further illustrates an optic zone (701), a lens diameter (702) and a plurality of non-refractive features of the contemplated design (703).

In this exemplary example, the lens diameter is approximately 14 mm, the optic zone is designed substantially with single vision refractive power and is approximately 8 mm in diameter, and the non-refractive features are arranged in the form of borders of the multiple circular apertures within the optic zone and are approximately 1 mm in diameter each. The borders of these non-refractive features (703) arranged in the form of multiple circular apertures may be configured to be between completely opaque and substantially opaque. For example, the transmission properties of the non-refractive features, the borders of the multiple circular apertures in this example, may be configured such that the >95% of the light incident on the non-refractive feature or border is absorbed or not transmitted.

The width of the border of the multiple circular apertures, i.e. non-refractive features, contemplated in FIG. 7 is approximately 50 μm (704). It is magnified relative to the size of the contact lens described herein to demonstrate and improve legibility of the feature. The remainder of the optic zone devoid of the contemplated non-refractive features, including the transparent area within the plurality of apertures, contains the single vision design matching the basic prescription of the wearer.

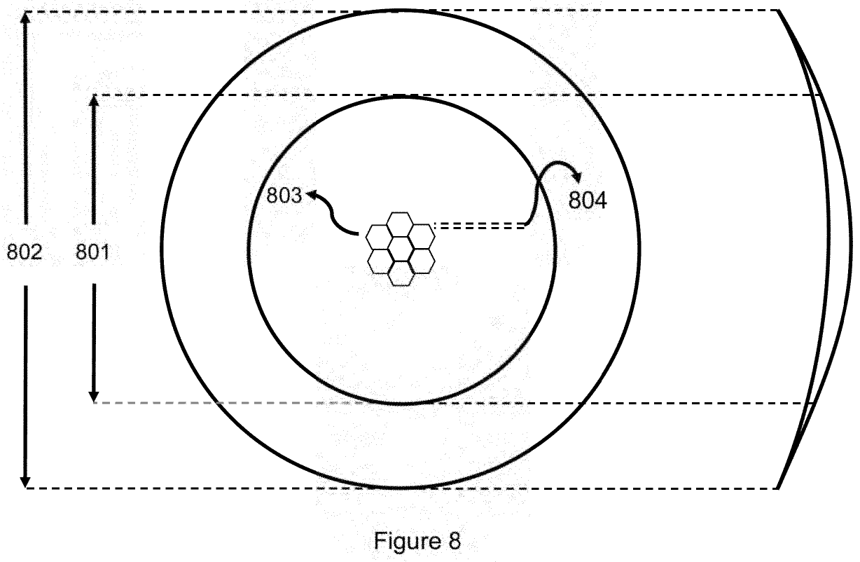

FIG. 8 shows the frontal view and cross-sectional view of another exemplary contact lens embodiment, not to scale. The frontal view of the exemplary contact lens embodiment further illustrates an optic zone (801), a lens diameter (802) and a plurality of conjoined hexagonal shaped non-refractive features (803) of the contemplated design. In this exemplary example, the lens diameter is approximately 14.2 mm, the optic zone designed substantially with single vision refractive power is approximately 9 mm in diameter, and the non-refractive features arranged in the form of the borders of multiple hexagonal apertures within the optic zone are approximately 1 mm in maximum diameter each.

The borders of these non-refractive features arranged in the form of multiple hexagonal apertures (803) may be configured to be between completely opaque or translucent. For example, the transmission properties may be configured such that the >90% of the light incident on the non-refractive feature or border is absorbed or not transmitted.

The width of the border of the multiple hexagonal apertures, i.e. non-refractive features, contemplated in FIG. 8 is approximately 25 μm (804). It is magnified relative to the size of the contact lens described herein to demonstrate and improve legibility of the feature. The remainder of the optic zone devoid of the contemplated non-refractive features, including the transparent area within the plurality of apertures, contains the single vision design matching the basic prescription of the wearer.

In yet another contact lens embodiment, the plurality of non-refractive features may be arranged, as the borders of a plurality of circular, semi-circular, elliptical, or hexagonal, or any other polygon shaped, apertures; wherein the plurality includes at least 2, 3, 5, 7, 9, 12 or 15 non-refractive features.

In some other contact lens embodiment, the number of non-refractive design features arranged in form of the borders of the plurality of polygonal shaped apertures may be between 4 and 7, or between 3 and 9, or between 2 and 12, or between 3 and 15. In some embodiments, the non-refractive design features arranged in form of the borders of the plurality of apertures may be separated, while they may be adjoined, or conjoined, in other embodiments.

In yet another contact lens embodiment, the non-refractive features configured as the borders of multiple apertures or multiple regions, or multiple zones, or multiple segments may be arranged within the central 1, 2, 3, 4, 5 or 6 mm of the optic zone of the contact lens. In yet another contact lens embodiment, the non-refractive features configured as the borders of multiple apertures or multiple regions, or multiple zones, or multiple segments may be arranged between the central 1 mm and 3 mm, or the central 2 mm to 4 mm, or the central 3 mm to 5 mm or the central 2 mm to 6 mm of the optic zone of the contact lens, as disclosed herein.

In certain contact lens embodiments, the width of the completely opaque, substantially opaque, or translucent, border of the contemplated non-refractive design features within the optic zone of the contact lens may be at least 5 μm, 10 μm, 20 μm, 30 μm, 40 μm or 50 μm in width. In certain contact lens embodiments, the width of the opaque border of the contemplated design features within the optic zone of the contact lens may be between 5 and 15 μm, 15 and 25 μm, or 10 and 50 μm in width.

In some other embodiment, the border of the contemplated design features within the optic zone of the contact lens may be opaque and yet in some other embodiments, the border of the contemplated design features may be translucent. In some embodiments, the width of the border or design feature may not be constant across multiple apertures. The shapes of the multiple apertures may also be different within one embodiment of the present disclosure.

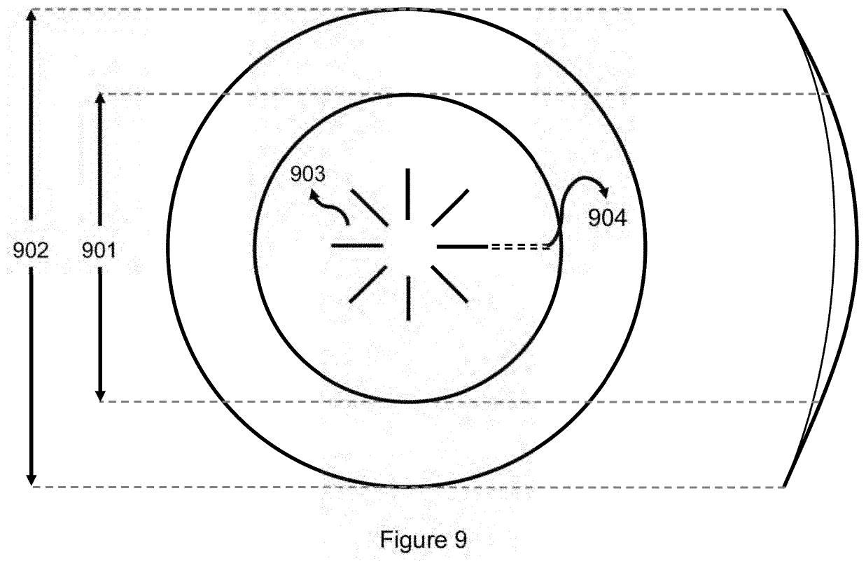

FIG. 9 shows the frontal view and cross-sectional view of another exemplary contact lens embodiment, not to scale. The frontal view of the exemplary contact lens embodiment further illustrates an optic zone (901), a lens diameter (902) and a plurality of non-refractive features (903) of the contemplated design.

In this exemplary example, the lens diameter is approximately 14.5 mm, the optic zone designed substantially with single vision refractive power is approximately 8 mm in diameter, and the non-refractive features, configured as line segments, or striae, are approximately 2 mm in length. These non-refractive features (903) may be substantially opaque; wherein 95% of the light incident on the non-refractive features is not transmitted or absorbed.

The width of the non-refractive features (904) contemplated in FIG. 9 is approximately between 25 μm and 50 μm, it is only magnified in the figure to demonstrate the feature relative to the size of the contact lens described herein. In a preferred embodiment, the maximum width of the non-refractive features is not to exceed 100 μm, 150 μm, or 200 μm to avoid unwarranted consequential effects on the resolution characteristics. The remainder of the optic zone devoid of the contemplated non-refractive features, including the transparent area within the plurality of apertures, contains the single vision design matching the basic prescription of the wearer.

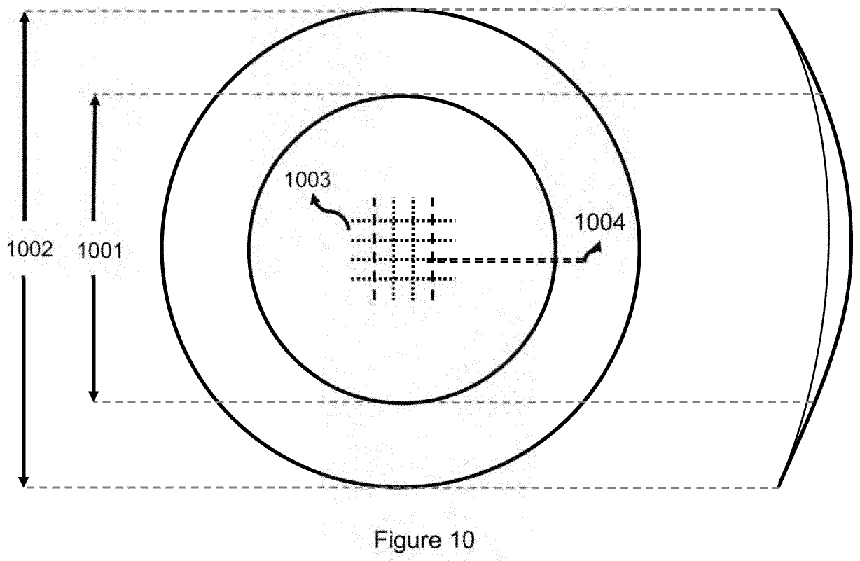

FIG. 10 shows the frontal view and cross-sectional view of another exemplary contact lens embodiment, not to scale. The frontal view of the exemplary contact lens embodiment further illustrates an optic zone (1001), a lens diameter (1002) and the non-refractive feature (1003).

In this example, the lens diameter is approximately 14 mm in diameter, the optic zone designed substantially with single vision refractive power and is approximately 8 mm in diameter. The contemplated design feature of this embodiment is a grid pattern, positioned in the centre of the contact lens spanning about 3 mm in height and width. The borders of these grid lines (1003) may be configured completely opaque or substantially opaque. The width of the non-refractive features (1004) contemplated in FIG. 10 is approximately between 50 μm and 100 μm, it is only magnified in the figure to demonstrate the feature relative to the size of the contact lens described herein.

The embodiment of FIG. 10 may also be configured in other variations, for example, the contemplated non-refractive design features within the optic zone may be at least 5 μm, 10 μm, 20 μm, 30 μm, 40 μm or 50 μm in width. The embodiment of FIG. 10 may also be configured in other variations, for example, the contemplated non-refractive design features within the optic zone may be between 5 and 15 μm, 15 and 25 μm, or 10 and 50 μm in width. In a preferred variation of the embodiment of FIG. 10, the maximum width of the non-refractive features, i.e. width of the lines forming a grid pattern, does not to exceed 150 μm, 200 μm or 250 μm to avoid unwarranted consequential effects on the resolution characteristics of the eye.

In other embodiments, the contemplated non-refractive design feature may be positioned in the located in periphery of the optic zone. In yet another contact lens embodiment, the number of fine lines, or striae, forming the grid pattern, may be at least 5, 9, 15 or 25. In some other contact lens embodiment, the number of design features, lines or striae, forming the grid pattern, may be between 5 and 9, or between 9 and 15, or between 9 and 15, or between 5 and 25. In one another embodiment, only one long substantially unbroken curvilinear line or a zig-zag line may be contemplated to run through the optical zone with a length of at least 3 mm, 6 mm, 9 mm, or 12 mm.

In yet another contact lens embodiment, the one or more striations may be arranged in a symmetric or random fashion, they may be centric with the optical axis or decentred. Striations may also consist of straight or curved lines, they may touch or cross each other, or all be placed in isolation, or a combination thereof. Striations may vary in width and length. There may be different patterns applied to lenses worn in the left and right eye.

In yet another contact lens embodiment the contemplated design features (i.e. a plurality of striations or moiré pattern) within the optic zone of the contact lens may be separated from each other. In yet another embodiment, the contemplated plurality of non-refractive features may be configured adjacent to or interlaced with each other.

Due to the natural blink facilitated by the combined action of the upper and lower eyelids the contact lens may freely move with respect to the pupil of the wearer. This may result in a temporally varying stimulus, which may further boost the inhomogeneity artificially introduced into the visual imagery, to reduce the rate of progression in a myopic wearer.

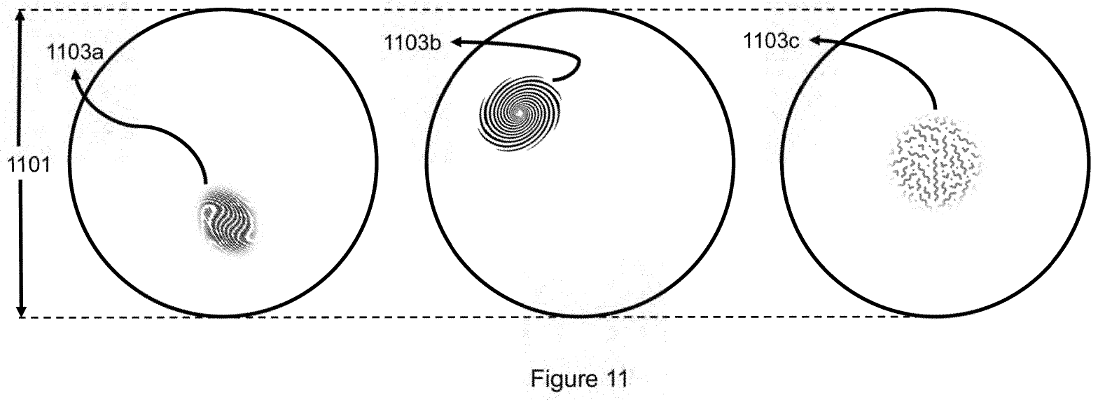

FIG. 11 shows the frontal view of three additional exemplary contact lens embodiments, not to scale. The frontal view of the exemplary contact lens embodiment illustrates only the zoomed-in view of the optic zone (1101), and three contemplated non-refractive design features (1103a, 1103b, and 1103c). In this example, the non-refractive design feature (1103a) is a representative example of the contemplated moiré pattern, which is configured away from the centre of the contact lens embodiment.

The non-refractive design feature (1103b) illustrates another representation of the contemplated curvilinear pattern across the optic zone; which assumes a spiral pattern. The non-refractive design features (1103c) illustrate a Memphis pattern centred about the optical centre of the contact lens. The optic zone is designed substantially with single vision refractive power and is approximately 8 mm in diameter. The width of the design features range between 5 to 100 μm, the substantially opaque features in the figure are highlighted to demonstrate the features relative to the size of the contact lens described herein.

In yet another contact lens embodiment, the designed feature (i.e. a plurality of non-refractive striations or moiré pattern) may be contained within the central 1, 2, 3, 4, 5 or 6 mm of the optic zone of the contact lens. In yet another contact lens embodiment, the design feature (i.e. plurality of non-refractive striations or moiré pattern) may be contained between the central 1 mm and 3 mm, or the central 2 mm to 4 mm, or the central 3 mm to 5 mm or the central 2 mm to 6 mm of the optic zone of the contact lens. In yet another contact lens embodiment the contemplated design features (i.e. a plurality of striations or moiré pattern) within the optic zone of the contact lens may be separated from each other. In yet another embodiment, the contemplated plurality of non-refractive features may be configured adjacent to or interlaced with each other. In certain contact lens embodiments, the width of the contemplated design features (i.e. a plurality of striations or moiré pattern) within the optic zone of the contact lens may be at least 5 μm, 10 μm, 20 μm, 30 μm, 40 μm or 50 μm in width.

In certain contact lens embodiments, the width of the contemplated design features within the optic zone of the contact lens may be between 5 and 15 μm, 15 and 25 μm, or 10 and 50 μm in width. In some other embodiment, the border of the contemplated design features within the optic zone of the contact lens may be opaque and yet in some other embodiments, the border of the contemplated design features may be translucent. In some embodiments, the width of the design feature may not be constant across a plurality of non-refractive features.

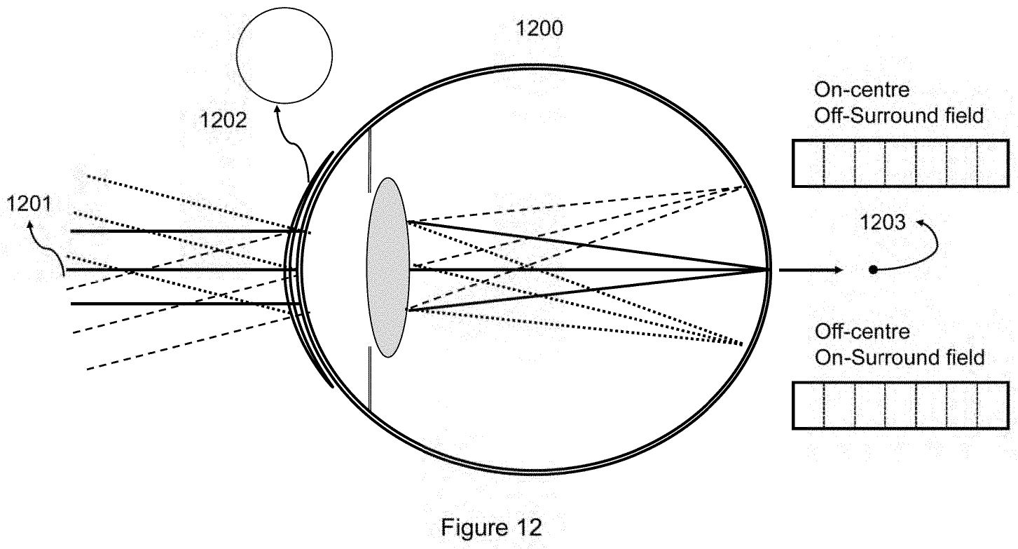

FIG. 12 shows the schematic diagram depicting incoming light, of a visible wavelength, for example, 555 nm, with a vergence 0 D, from a wide-angle field of view (1201) entering a 2 D myopic model eye (1200), which is corrected with a standard single vision lens of prior art (1202).

The retinal ganglion cell activity recorded by the on-centre/off-surround and off-centre/on-surround circuitry (1203), when the standard single vision lens of prior art (1202) moves on the anterior surface of the eye either due to natural blink action, or due to habitual eye movements, or combination thereof, demonstrating or showing minimal retinal activity or retinal activity at a basal rate. The relative difference in the temporal integration of on- and off-receptive field activity determines further eye growth.

The present disclosure postulates that an inactive retina triggers eye growth and an active retina reduces the growth or triggers a stop signal. The present disclosure further contemplates that a standard single vision contact lens or a spectacle lens of the prior art and/or a spatially homogenous visual imagery contributes to a homogeneous and substantially edge-less visual imagery leaving the retina in a baseline state (i.e. baseline or constant firing pattern of the retinal ganglion cells) and thus promoting further eye growth leading to more myopia.

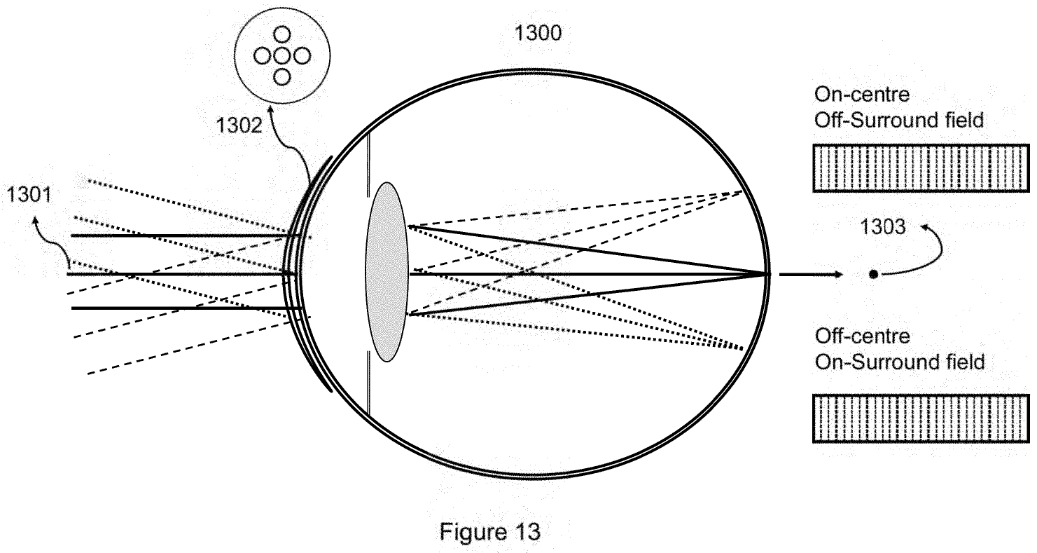

FIG. 13 shows the schematic diagram depicting an incoming bundle of light, of a visible wavelength, for example 555 nm, with a vergence 0 D, from a wide-angle field of view (1301) entering a 2 D myopic model eye (1300), corrected with one of the exemplary embodiments (1302) disclosed herein. The retinal ganglion cell activity recorded by the on-centre/off-surround and off-centre/on-surround circuitry (1303), when the exemplary embodiment (1302) moves on the anterior surface of the eye either due to natural blink action, or due to habitual eye movements, or combination thereof, demonstrating or showing increased activity at the retina compared to the baseline state.

A simple model eye was chosen for illustrative purposes in FIGS. 12 and 13, However, in other embodiments, schematic raytracing model eyes like Liou-Brennan, Escudero-Navarro and others may be used instead. The examples provided herein have used a 2 D myopic model eye to disclose the present invention, however the same disclosure can be extended to other degrees of myopia, i.e. −1 D, −3 D, −5 D or −6 D. Further, it is understood that one can draw extensions to eyes with varying degrees of myopia in conjunction with astigmatism. In the embodiments, reference was made to a specific wavelength of 555 nm, however it is understood that one can draw extensions to other visible wavelengths between 420 nm and 760 nm.

Modelling of various exemplary contact lens embodiments (D1 to D7) demonstrate that the contemplated non-refractive features used in conjunction with the single vision optical profiles provide an increase in the retinal ganglion cell activity, gauged by the increase in the average retinal spike rate obtained using the virtual retinal platform disclosed herein.

In other embodiments, various other surrogate measures of retinal ganglion cell activity may be considered, for example, inspection of the spike train analysis for the neuronal bundle of choice.

To demonstrate the workings for the contact lens embodiments in accordance with the invention, advanced optical modelling experiments were conducted using two different types of contact lenses for each test case (i.e. Examples 1 to 7) as described herein. The first type included single vision control contact lenses (C1 to C7) that were matched to the base prescription of the schematic model eyes, to provide correction of the refractive error, emulating the standard of care. The second type included various exemplary contact lens embodiments (D1 to D7), which are essentially the same single vision, standard of care, control contact lens (C1 to C7) which are further configured with additional non-refractive features, designed in accordance with the invention.

To demonstrate the workings of the invention, the control (C1 to C7) and exemplary embodiment contact lenses (D1 to D7) were fitted, tested/evaluated, one at a time, on the modified schematic model eyes described in Examples 1 to 7, respectively. For the purpose of demonstrating the workings of these Examples 1 to 7, only the optic zone (8 mm) of the contact lens was modelled. In other examples, the entire contact lens including the peripheral zone and edge may be modelled as desirable.

The surface transmission properties of the front surface of the contact lens were modified to design the features of Examples 1 to 7. The transmission is computed as a fraction of 100%, with 100% meaning that all of the light is transmitted as if there were no absorption, reflection, or vignetting losses. In certain embodiments of the present disclosure, surface transmittance was defined as the relative arbitrary fraction of intensity that the ray transmits through the surface. In some other embodiments of the disclosure, the relative arbitrary fraction of intensity may be configured to be wavelength dependent. In certain other embodiments of the disclosure, the arbitrary fraction of intensity may be configured to be polarisation sensitive.

To evaluate the simulated retinal ganglion cell activity, the contact lens was slid on the anterior corneal surface in various decentration positions mimicking on-eye lens movements with blink in the vertical direction and/or the relative lens movement that may be caused with saccadic eye movements in the horizontal direction). The contact lens movements with respect to the centre of the anterior corneal surface were contained between +/−1 mm in both horizontal and vertical directions. To emulate the on-eye movement of the contact lens, both decentration and tilt functions were used in the modelling apparatus.

At each of the decentred lens positions, a wide-field retinal image simulation was performed. Forty-eight (48) such simulated retinal images constituted the input stream for the virtual retinal platform to yield retinal ganglion cell activity. In this example, each of the 48 image frames were configured to be 50 milliseconds accounting for 2.4 seconds of real time stimulation presentation for the virtual retinal model. Each frame of the input stream was configured over 512×512 pixels, wherein each frame was configured to cover the entire diameter of the circular neuron region, encompassing a region approximately, 5°×5° (foveal) or 15°×15° (macular) of the retina of the virtual retinal platform. The bit depth for each pixel in the input stream was digitised to range from 0 to 255 (i.e. 8-bit). Specific retinal settings and configurations, described in Equations 1 to 9, used to demonstrate the workings for the contact lens embodiments of the present disclosure are discussed in the following section.

In all the Examples 1 to 7, the outer plexiform layer was configured to have a centre region subtending approximately 1.5° (i.e. σC of Equation 2) and a surround region subtending approximately 4.75° (i.e. σS of Equation 3). The centre and surround temporal scales of the outer plexiform layer were set to approximately 1 milli-seconds, represented by variables τC and τS of Equations 2 and 3, respectively. The variables governing the integration centre-surround signals, as described in the Equation 1 herein, were chosen to be w_{OPL}=1 and λ_{OPL}=10. The static nonlinearity coefficients of the bipolar and ganglion cell synapses were fixed across all the Examples 1 to 7. The bipolar linear threshold was set to 0, the linear threshold value was held constant at 80, and the bipolar amplification value held at 100.

The values for the neuronal model were maintained across all Examples 1 to 7, wherein a leak of 0.75, neuronal noise of 20, membrane capacitance of 150 and firing threshold of 2.4 were used for the simulations of Examples 1 to 7. The post-synaptic pooling variable Sigma was ignored. The options for a contrast gain control mechanism, utility of the supplementary high-pass filter of the outer plexiform layer and the utility of lateral connectivity of the amacrine cells were kept variable across the Examples 1 to 7. Further details of the specific setting used are disclosed herein.

### Example 1—Control (C1) and Exemplary Embodiment (D1) Designs

In this example, the following parameters of the schematic model eye of Table 1 were altered to configure a 1D myopic eye (i.e. base prescription Rx of −1 D) in its 2D accommodated state; (1) the anterior lens radius of curvature (R=8.22 mm); and (2) the anterior lens conic constant (Q=−2.314). The model was configured to focus on a near object approximately 50 cm away from the eye. The modified myopic schematic model eye was corrected, one at a time, with the control (C1) and exemplary embodiment (D1) contact lenses. The control contact lens C1 was modelled using a front surface radius (R=7.936 mm, Q=−0.221), a centre thickness (0.135 mm), a back surface radius (R=7.75 mm, Q=−0.25) and refractive index of 1.42. The control contact lens C1 is free/devoid of any non-refractive features contemplated in this present disclosure.

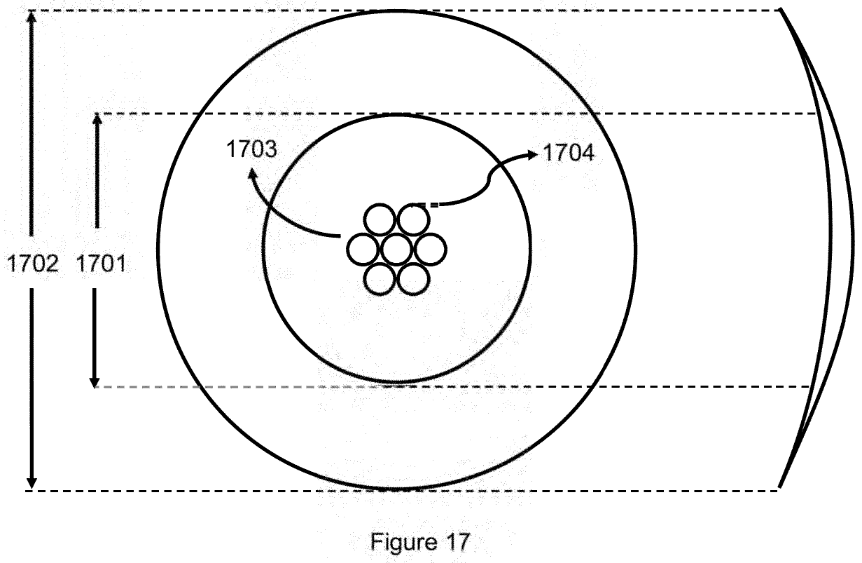

The exemplary embodiment (D1) contact lens is a single vision contact lens with same optical design as control (C1) which was further configured with additional non-refractive features as disclosed in FIG. 17.

FIG. 17 shows the frontal view and cross-sectional view of the exemplary contact lens embodiment D1, not to scale. The frontal view of the exemplary contact lens embodiment further illustrates an optic zone (1701), a lens diameter (1702) and a plurality of non-refractive features (1703) comprising conjoined circular shaped non-refractive features of the contemplated design (D1). The total number of circular apertures is 7. The total dimension of the non-refractive feature comprising multiple apertures is approximately 3.75 mm in diameter. The dimensions of each aperture is approximately 1.25 mm in diameter. The width of the border of each of the aperture is approximately 100 μm (1704).

The non-refractive features are magnified relative to other features of the contact lens for identification and legibility. The remainder of the portion of the optic zone (1701) devoid of the non-refractive features of the exemplary embodiment D1 is configured with the underlying single vision prescription parameters matching the base prescription of the eye.

In this exemplary example D1, the lens diameter is approximately 14.2 mm, the optic zone designed substantially with single vision refractive power is approximately 8 mm in diameter, and the non-refractive features arranged in form of multiple circular apertures within the optic zone are approximately 1 mm in diameter each. The simulated retinal images were computed and analysed with the control C1 and embodiment D1 contact lens designs fitted, one at a time, on the schematic model eye of Example 1, following the steps disclosed in paragraphs [00271] to [00273].

In this Example 1, additional variables of the virtual retinal platform were contemplated with the following settings; the option of a contrast gain control mechanism, described in Equations 1, 5 and 6, were used with the following input parameter values: (i) an outer plexiform amplification λ_{OPL} value of 150 Hz per normalised luminance unit; (ii) bipolar inert leaks g_{A}⁰ of 5 Hz; (iii) the feedback amplification λ_{A} of 100 in Hz; (iv) spatial scale σA of 2.5°; and (v) temporal scale τA of 0.01 milli-seconds. The arrangement of the neuronal bundle (1402) was in a circular arrangement spanning across a 15°×15° field of view.

A sparse lateral connectivity mode of the virtual retina was used with 10 pre-synaptic neurons with 10% of positive weight and a weight variance of 0.01. Further, the supplementary high-pass filter option of outer plexiform layer, described in Equations 2 and 3, was not used. The post-synaptic pooling option was muted.

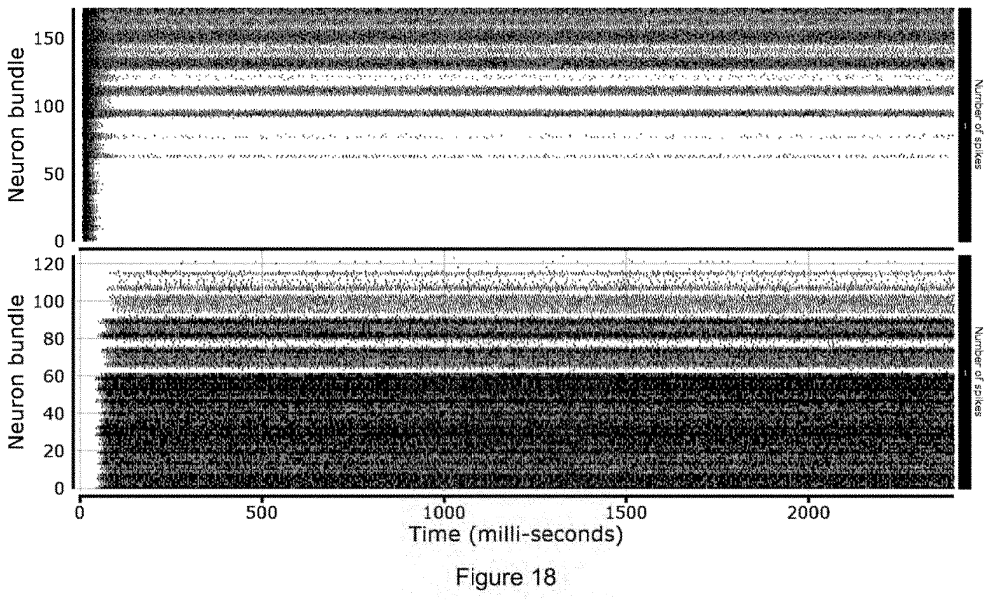

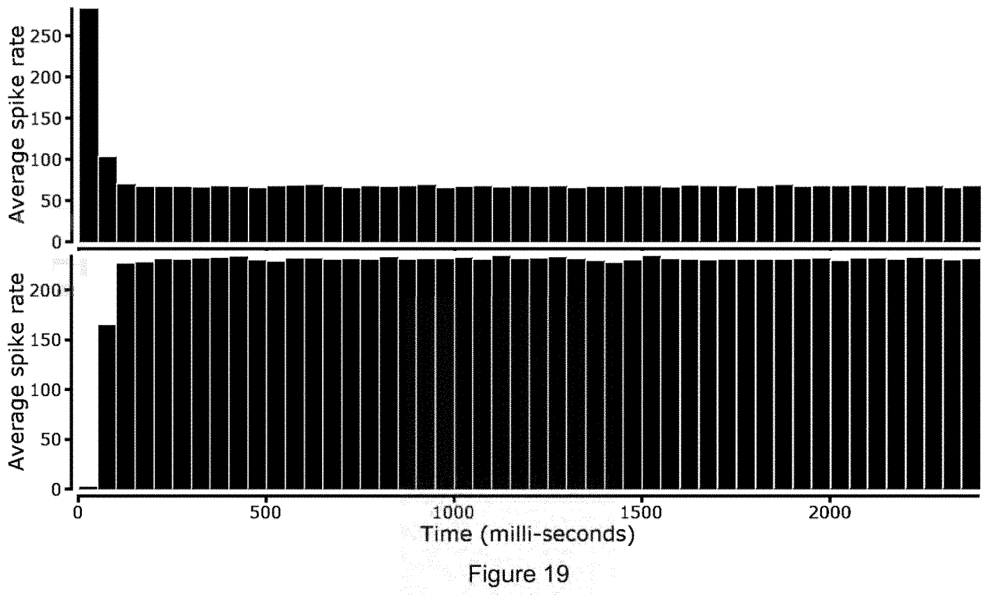

The postprocessing of the computed simulated retinal images of control (C1) contact lens design of Example 1, using the virtual retinal platform, as discussed herein, results in a spike train as a function of time (FIG. 18) and pen-stimulus histogram highlighting the average spike rate as a function of time (FIG. 19), for cells with both on- and off-polarities. The top and bottom sub-graphs of FIG. 18 and FIG. 19 represent data for on- and off-cells, respectively.

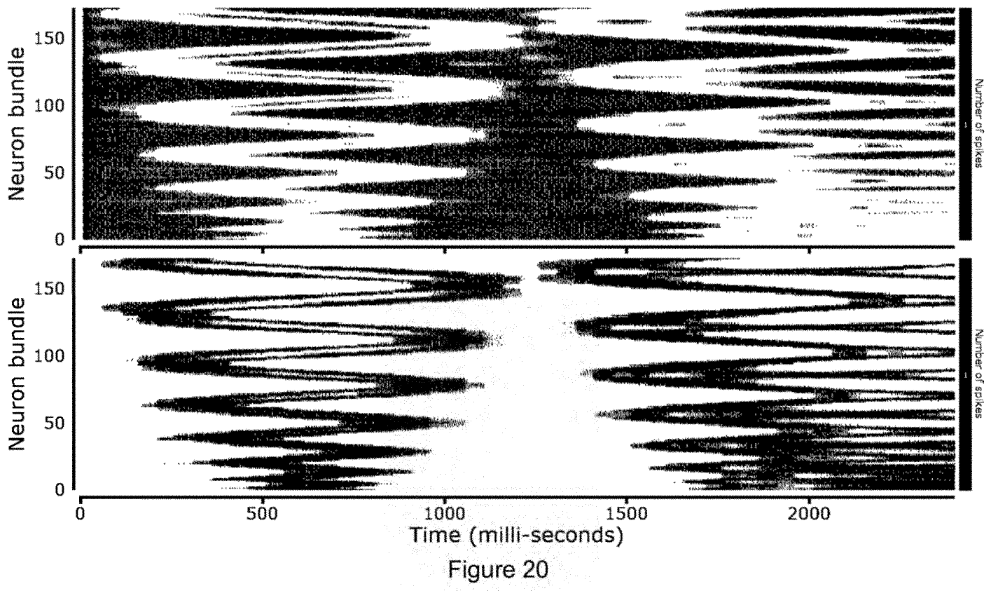

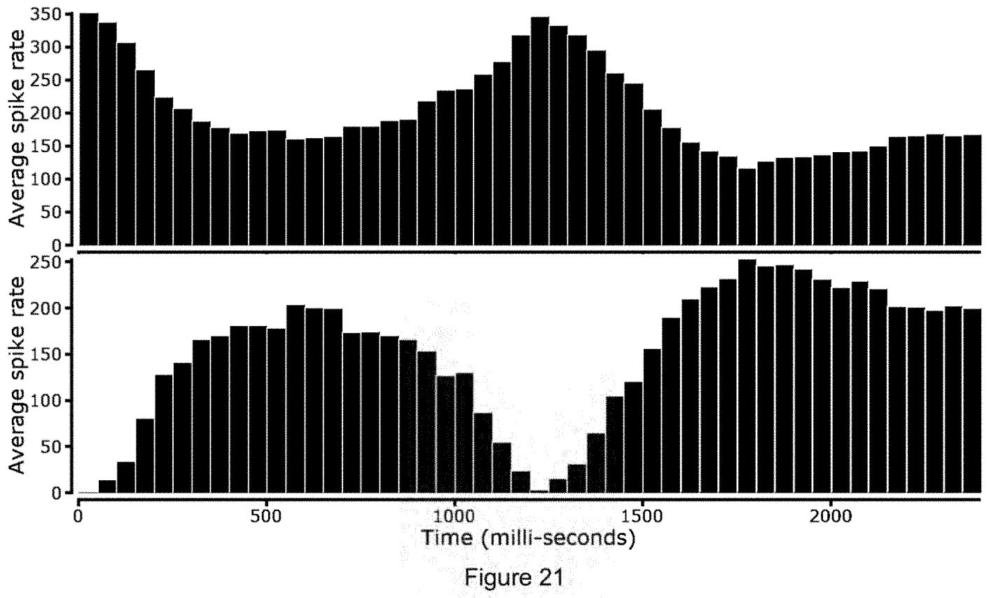

The postprocessing of the computed simulated retinal images of embodiment (D1) contact lens design of Example 1, using the virtual retinal platform, as discussed herein, results in a spike train as a function of time (FIG. 20) and peri-stimulus histogram highlighting the average spike rate as a function of time (FIG. 21), for cells with both on- and off-polarities. The top and bottom sub-graphs of FIG. 20 and FIG. 21 represent data for on- and off-cells, respectively.

The neuronal activity with control (C1) contact lens, depicted as spike trains of FIG. 18, is time invariant or monotonous as a function of time, for cells with both type of polarities, on- and off.

On the other hand, the neuronal activity with embodiment (D1) contact lens, depicted as spike trains of FIG. 20, is time variant, or non-monotonous as a function of time, for cells with both type of polarities, on- and off.

In the Example 1, the neuronal activity with control (C1) contact lens, depicted as average spike rate of FIG. 19, follows a monotonous profile following the initial 100 milliseconds signifying a stabilisation of the signal. This observed pattern is similar for cells with both type of polarities, on- and off. In the Example 1, the average spike rate with control (C1) contact lens, following the initial 100 milli-second stabilisation period, for on-type cells is approximately a quarter (¼th) in magnitude to that obtained with off-type cells, as disclosed herein (FIG. 19). On the other hand, the neuronal activity with embodiment (D1) contact lens, depicted as average spike rate of FIG. 21, is time variant or non-monotonous as a function of time.

In this Example 1, the average spike rate for on-type cells obtained with embodiment (D1) contact lens is generally at least 3 to 4 times that obtained for on-type cells with the control (C1) contact lens. In this example, the average spike rate as function of time, described in FIG. 21, obtained with embodiment (D1) contact lens follows a quasi-sinusoidal pattern for both on-type and off-type cells.

The non-stationarity and non-linearity in the spiking responses obtained with the embodiment lens is attributed to the artificial edges, or luminous contrast profiles, in the retinal imagery, or temporal variation of the artificial edges.

In this Example 1, the on- and off-axis evaluation of the optical performance, was modelled in polychromatic mode, spanning 470 nm to 650 nm, using a luminosity function describing average spectral sensitivity of human visual perception of brightness in photopic condition at 4 mm pupil analysis diameter.

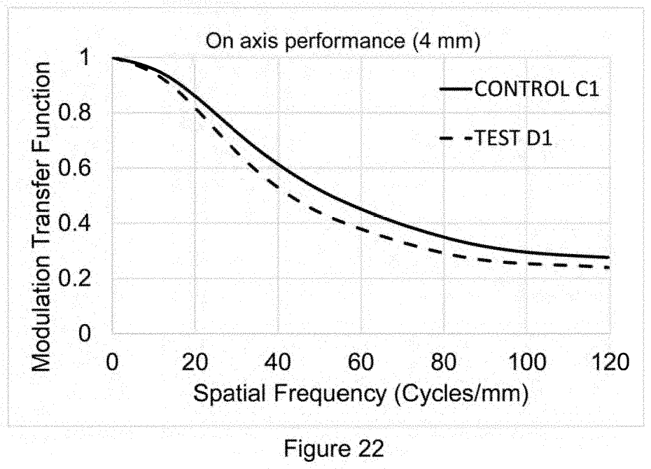

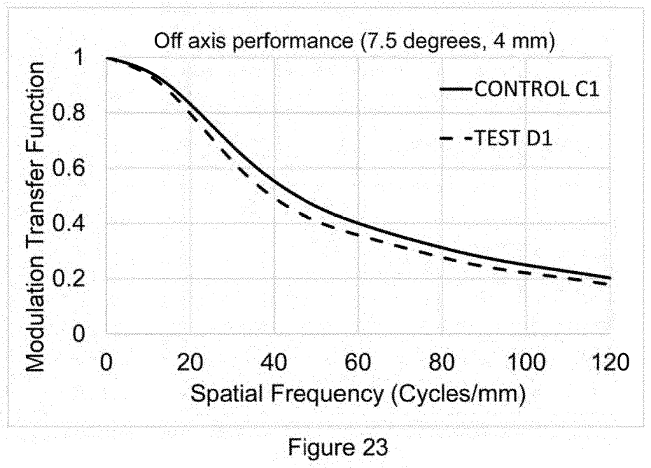

As described herein FIGS. 22 and 23, the wide-field optical performance, as gauged using modulation transfer function as function of spatial frequencies at 4 mm pupil diameter, between the control (C1) and exemplary embodiment (D1) contact lenses is significantly similar, i.e. with less than 5% variation in the area under the curves represented by the solid black line and dashed black lines. For the off-axis performance, in Example 1, the field of view considered for evaluation of performance was 15°, which is. ±7.5° from the centre.

### Example 2—Control (C2) and Exemplary Embodiment (D2) Designs

In this example, the following parameters of the schematic model eye of Table 1 were altered to represent a 2 D myopic eye with 1 DC astigmatism (i.e. base prescription Rx of −2D/−1DC), in its 2 D accommodated state; (i) the anterior corneal radius (Rₓ=7.829 mm) along the X-axis; (ii) the anterior corneal conic constant (Qₓ=−0.604) along the X-axis; (iii) the vitreous chamber depth of 17.339 mm; (iv) the anterior lens radii (R=8.22 mm); and (v) anterior lens conic constant (Q=−2.314). The model was configured to focus on a near object approximately 50 cm away from the eye. The modified myopic schematic model eye of was corrected, one at a time, with the control (C2) and exemplary embodiment (D2) contact lenses.

The control (C2) contact lens represents a single vision toric modelled using the following parameters: a front surface (R=8.226 mm, Q=−0.392), a centre thickness (0.135 mm), a toric back surface (R_{y}=7.75 mm, Q_{y}=−0.25; Rₓ=7.829 mm, Qₓ=−0.604) and a refractive index of 1.38. The control contact lens C2 is free/devoid of any non-refractive features contemplated in this present disclosure.

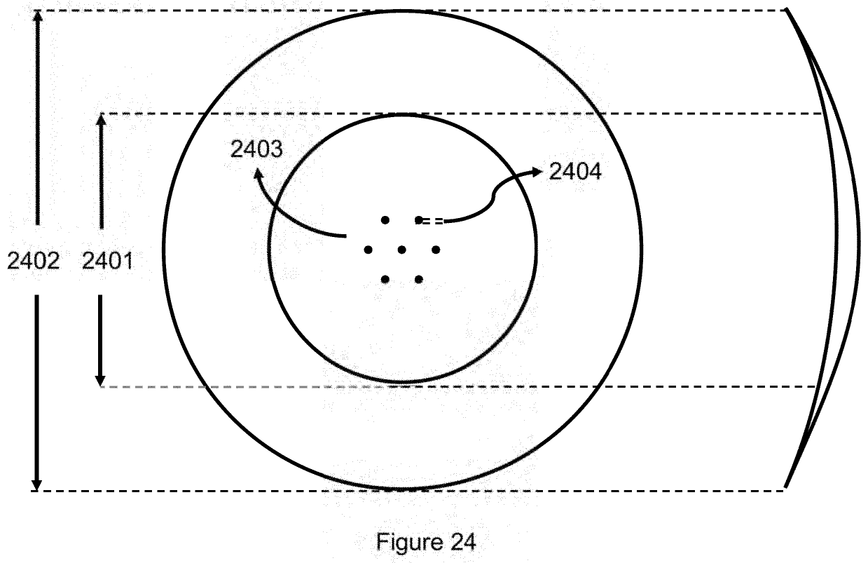

The exemplary embodiment contact lens (D2) is a single vision toric with same optical design as control C2 that was further configured with additional non-refractive features disclosed in FIG. 24.

The non-refractive features of the exemplary embodiment example D2 comprises a pattern of dots (2403), including a plurality of dots arranged in a hexagonal arrangement. This random pattern (2403) is positioned within the optical zone (2401) about the optical centre of the contact lens (2402). The total number of dots is 7. The total dimension of the dot pattern is approximately 3.5 mm in diameter. The dimensions of each dot in the dot pattern is approximately 125 μm in diameter (2404).

The non-refractive features are magnified relative to other features of the contact lens for identification and legibility. The remainder of the portion of the optic zone (2401) devoid of the non-refractive features of the exemplary embodiment D2 is configured with the underlying single vision prescription parameters matching the base prescription of the eye.

The simulated retinal images were computed and analysed with control C2 and embodiment D2 contact lens designs when fitted on the schematic model eye of Example 2, following the steps disclosed in paragraphs [00271] to [00273].

In this Example 2, additional variables of the virtual retinal platform were contemplated with the following settings; the option of a contrast gain control mechanism, described in Equations 1, 5 and 6, were used with the following input parameter values: (i) an outer plexiform amplification λ_{OPL} value of 150 Hz per normalised luminance unit; (ii) bipolar inert leaks g_{A}⁰ of 5 Hz; (iii) the feedback amplification λ_{A} of 100 in Hz; (iv) spatial scale σA of 2.5°; and (v) temporal scale τA of 0.01 milli-seconds. The arrangement of the neuronal bundle (1402) was in a circular arrangement spanning 15°×15° field of view.

A sparse lateral connectivity mode of the virtual retina was used with 10 pre-synaptic neurons with 10% of positive weight and a weight variance of 0.01. Further, the supplementary high-pass filter option of outer plexiform layer, described in Equations 2 and 3, was used with the following parameters values: a temporal scale of 0.2 milliseconds and spatial scale of 0.5°. The post-synaptic pooling option was muted.

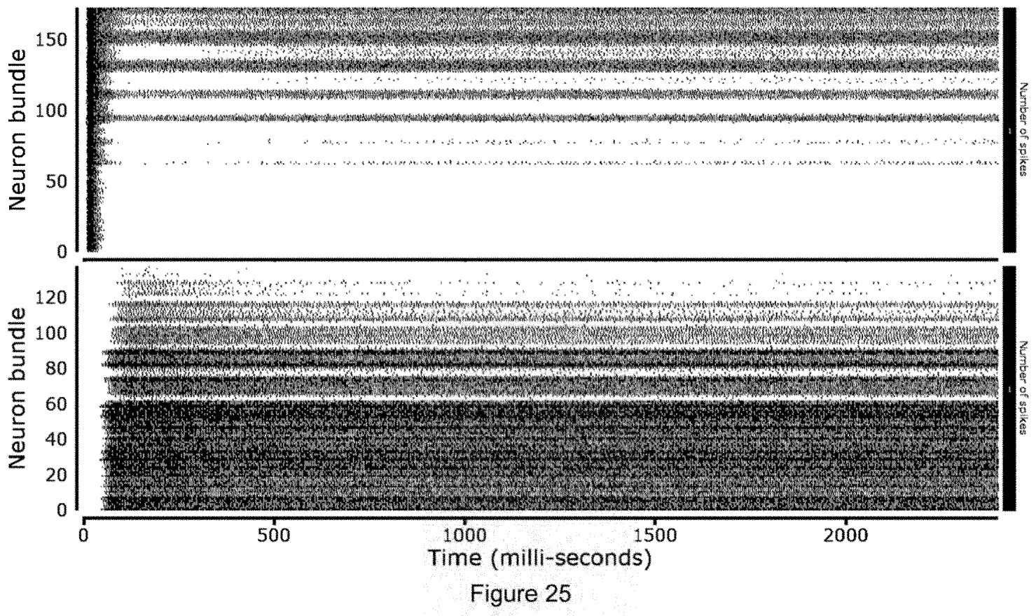

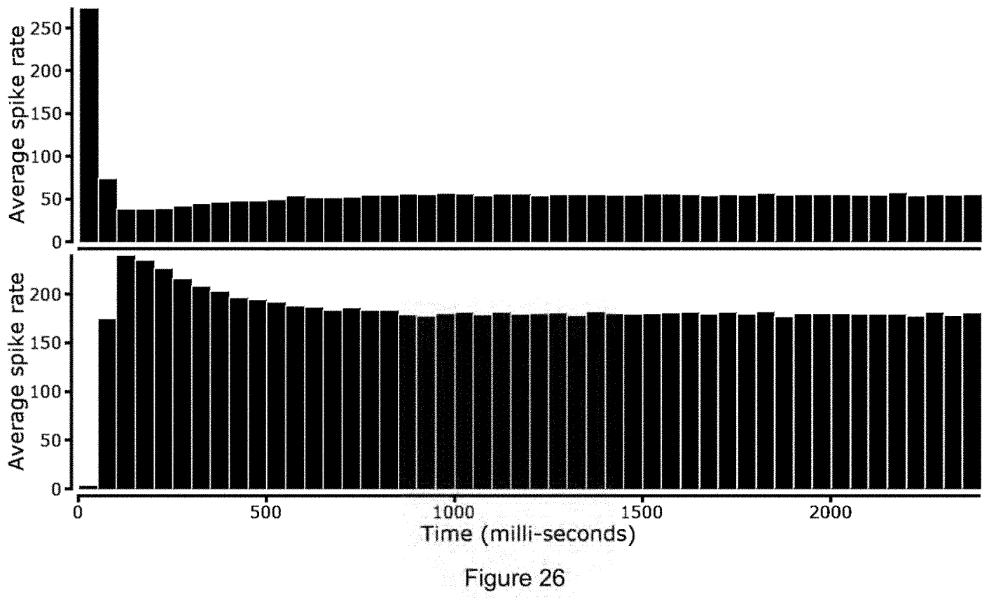

The postprocessing of the computed simulated retinal images of control (C2) contact lens design of Example 2, using the virtual retinal platform, as discussed herein, results in a spike train as a function of time (FIG. 25) and peri-stimulus histogram highlighting the average spike rate as a function of time (FIG. 26), for cells with both on- and off-polarities. The top and bottom sub-graphs of FIG. 25 and FIG. 26 represent data for on- and off-cells, respectively.

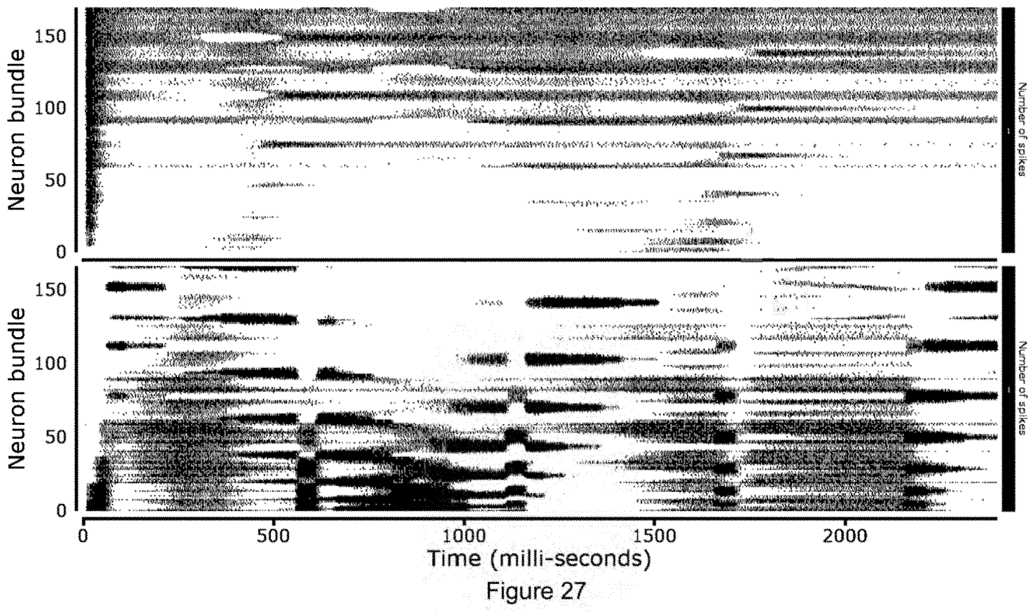

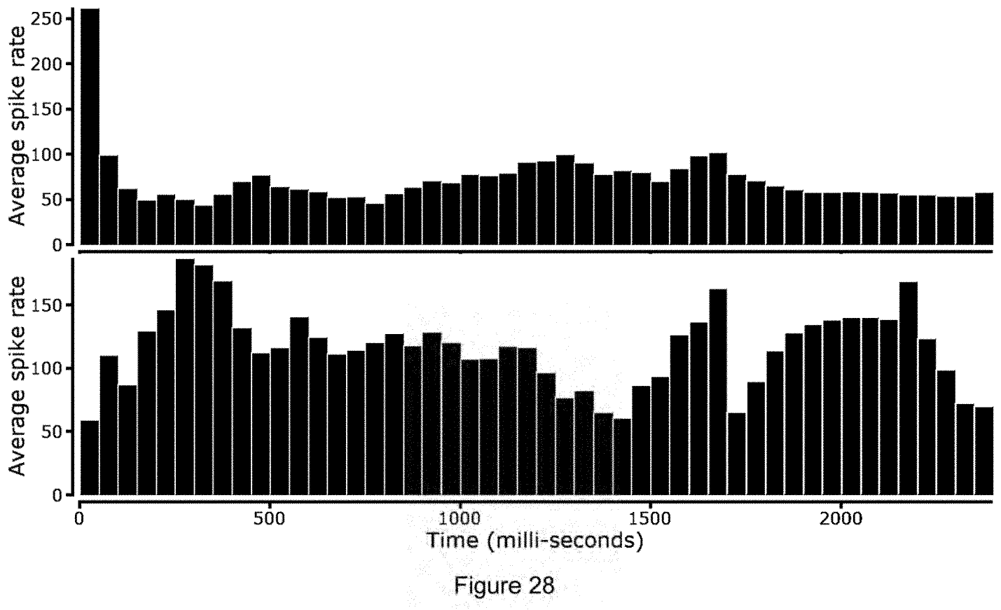

The postprocessing of the computed simulated retinal images of embodiment (D2) contact lens design of Example 2, using the virtual retinal platform, as discussed herein, results in a spike train as a function of time (FIG. 27) and peri-stimulus histogram highlighting the average spike rate as a function of time (FIG. 28), for cells with both on- and off-polarities. The top and bottom sub-graphs of FIG. 27 and FIG. 28 represent data for on- and off-cells, respectively.

The neuronal activity with control (C2) contact lens, depicted as spike trains of FIG. 25, is time invariant or monotonous as a function of time, for cells with both type of polarities, on- and off. On the other hand, the neuronal activity with embodiment (D1) contact lens, depicted as spike trains of FIG. 26, is time variant or non-monotonous as a function of time.

In the Example 2, the neuronal activity with control (C2) contact lens, depicted as average spike rate of FIG. 26, follows a linear profile bar the data of initial 150 milliseconds signifying stabilisation the signal. This observed pattern is similar for cells with both type of polarities, on- and off.

In the Example 2, the average spike rate, following the first 150 milli-second stabilisation period, for on-type cells is approximately a third ⅓^{rd} to ¼^{th} in magnitude to that obtained with off-type cells, as disclosed herein.

On the other hand, the neuronal activity with embodiment (D1) contact lens, depicted as average spike rate of FIG. 28, is time variant or non-monotonous as a function of time. However, the variation within spike rate as function of time obtained with embodiment D2 of Example 2 is lower in both amplitude and frequency when compared to the results obtained with embodiment D1 of Example 1.

In this Example 2, the average spike rate for on-type cells obtained with embodiment (D2) contact lens is generally at least 1.5 times that obtained for on-type cells obtained with control (D2) contact lens. In this example, the average spike rate as function of time, described in FIG. 28, obtained with embodiment (D2) contact lens follows a time-variant pattern for both on-type and off-type cells. The non-stationarity and non-linearity in the spiking responses obtained with the embodiment lens is attributed to the artificial edges, or luminous contrast profiles, in the retinal imagery, or temporal variation of the artificial edges.

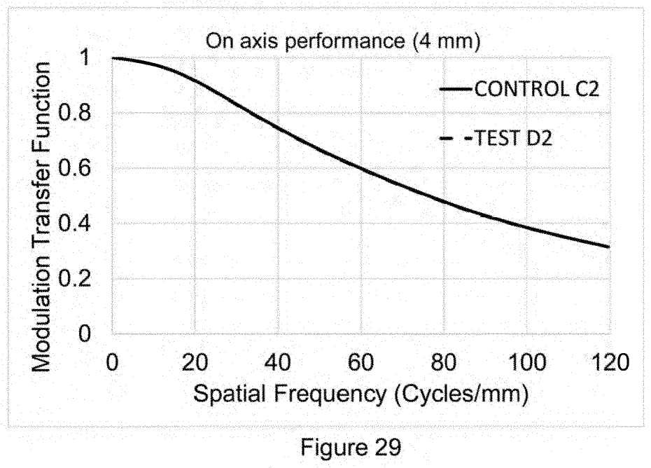

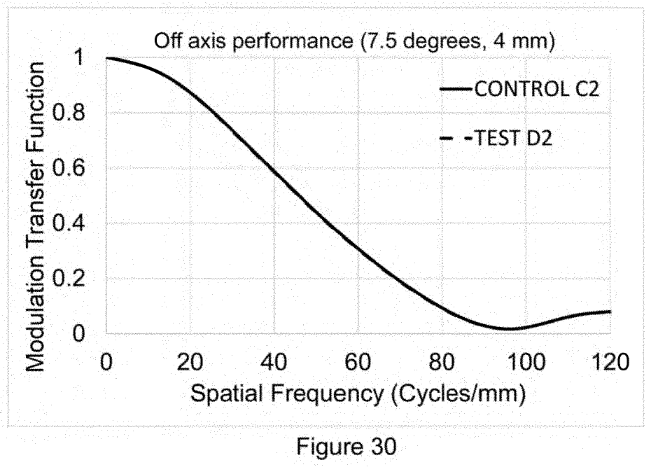

In this Example 2, the on- and off-axis evaluation of the optical performance, was modelled in monochromatic mode (589 nm) and at 4 mm pupil analysis diameter. As described herein FIGS. 29 and 30, the wide-field optical performance, as gauged using modulation transfer function as function of spatial frequencies at 4 mm pupil diameter, between the control (C2) and exemplary embodiment (D2) contact lenses is virtually indistinguishable. For the off-axis performance, in Example 2, the field of view considered for evaluation of performance was 15°, which is ±7.5° from the centre.

### Example 3—Control C3 and Exemplary Embodiment Design D3

In this Example 3, the following parameters of the schematic model eye of Table 1 were altered to represent a 3D myopic eye (i.e. base prescription Rx of −3D) in its unaccommodated state; (i) a vitreous chamber depth of 17.65 mm, and (ii) the retinal radius of curvature to 13.5 mm.

The model was configured to focus on a distant object approximately at optical infinity from the eye. The modified myopic schematic model eye of was corrected, one at a time, with the control (C3) and exemplary embodiment (D3) contact lenses. The control (C3) contact lens represents a single vision lens modelled using the following parameters: front surface (R=8.262 mm, Q=−0.137), a centre thickness (0.135 mm), a back surface (R=7.75 mm, Q=−0.25) and refractive index of 1.42. The control contact lens C3 is free/devoid of any non-refractive features contemplated in this present disclosure.

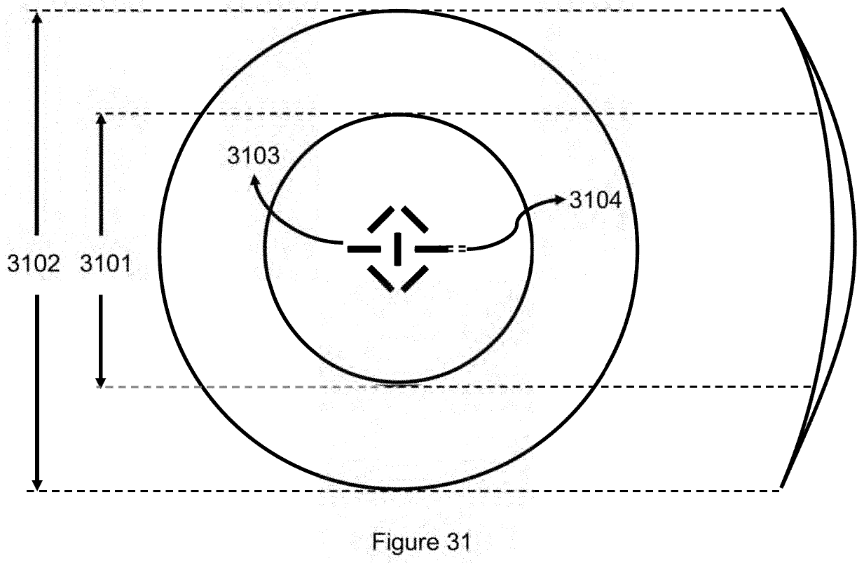

The second lens D3 represents the exemplary embodiment which is also a single vision contact lens with same parameters as control C3, that was further configured with non-refractive features disclosed in FIG. 31.

The non-refractive features of the exemplary embodiment example D3 (FIG. 31) comprises a random pattern of bars or thickened lines (3103), including a plurality of bars. This random pattern is positioned within the optical about the optical centre of the optic zone (3101) of the contact lens (3102). The total number of bars is 7. The total dimension of the grid pattern is approximately 4 mm in diameter. The dimensions of each bar in the random bar pattern is approximately between 50 μm×1.25 mm (3104).

The non-refractive features are magnified relative to other features of the contact lens for identification and legibility. The remainder of the portion of the optic zone (3101) devoid of the non-refractive features of the exemplary embodiment D3 is configured with the underlying single vision prescription parameters matching the base prescription of the eye.

The simulated retinal images were computed and analysed with control C3 and embodiment D3 contact lens designs when fitted on the schematic model eye of Example 3, following the steps disclosed in paragraphs [00271] to [00273].

In this Example 3, additional variables of the virtual retinal platform were contemplated with the following settings; the option of a contrast gain control mechanism, described in Equations 1, 5 and 6, were used with the following input parameter values: (i) an outer plexiform amplification λ_{OPL} value of 150 Hz per normalised luminance unit; (ii) bipolar inert leaks g_{A}⁰ of 5 Hz; (iii) the feedback amplification λ_{A} of 100 in Hz; (iv) spatial scale σA of 2.5°; and (v) temporal scale τA of 0.01 milli-seconds. The arrangement of the neuronal bundle (1602) was in a circular arrangement spanning 5°×5° field of view. A sparse lateral connectivity mode of the virtual retina was not used. Further, the supplementary high-pass filter option of outer plexiform layer, described in Equations 2 and 3, was used with the following parameters values: a temporal scale of 0.2 milliseconds and spatial scale of 0.5°. The post-synaptic pooling option was muted.

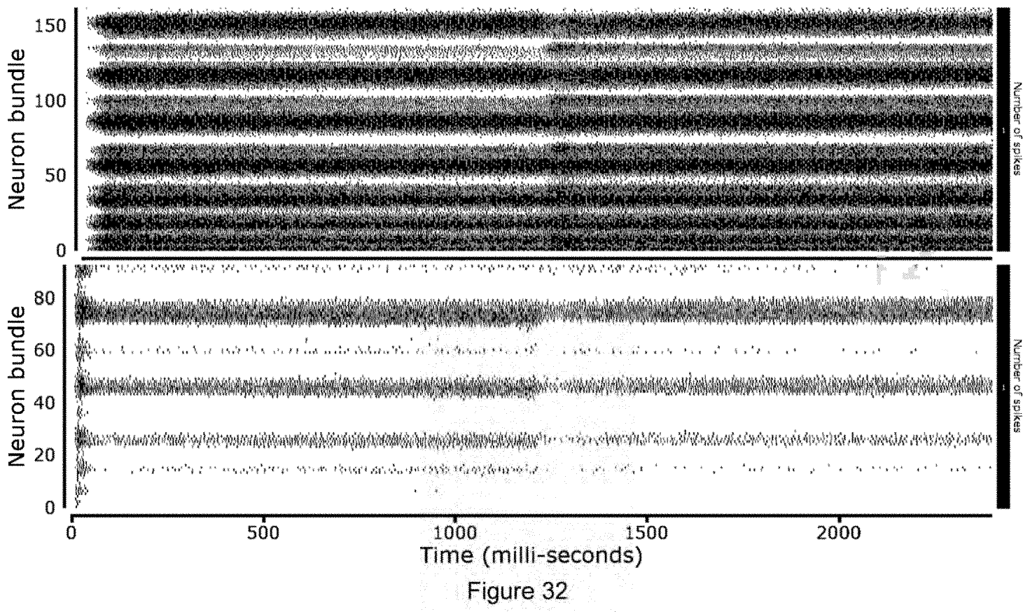

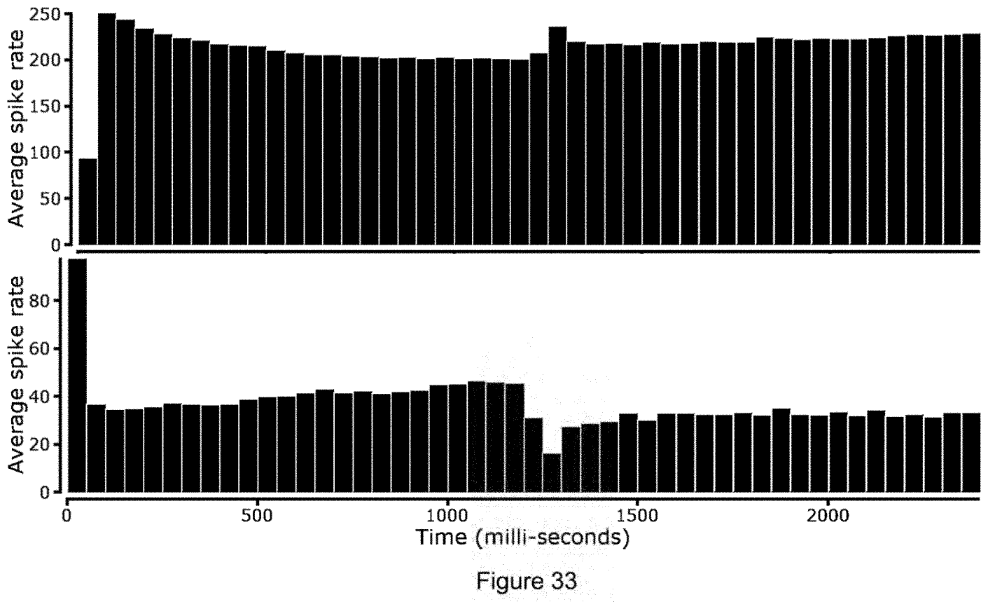

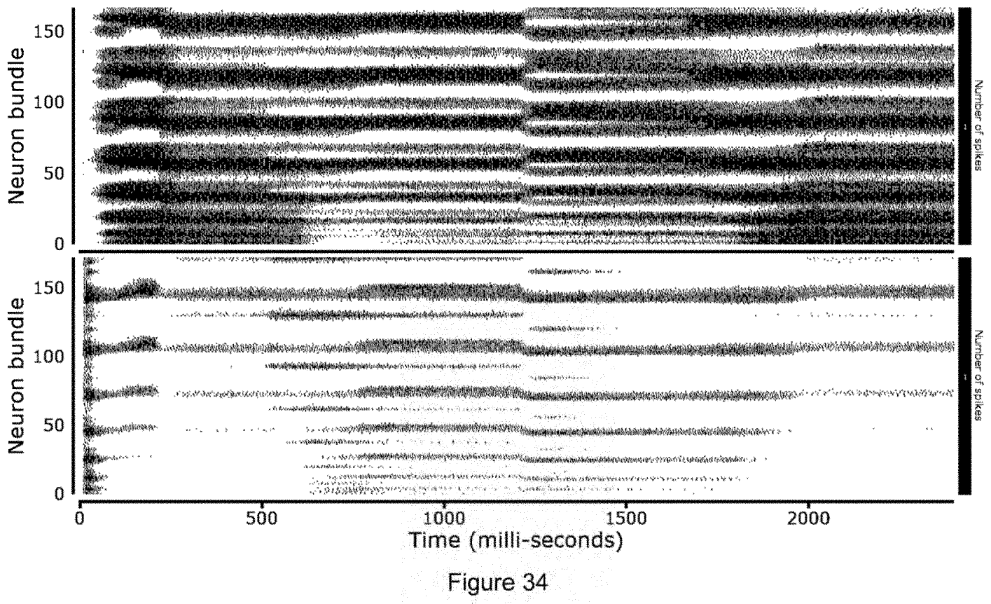

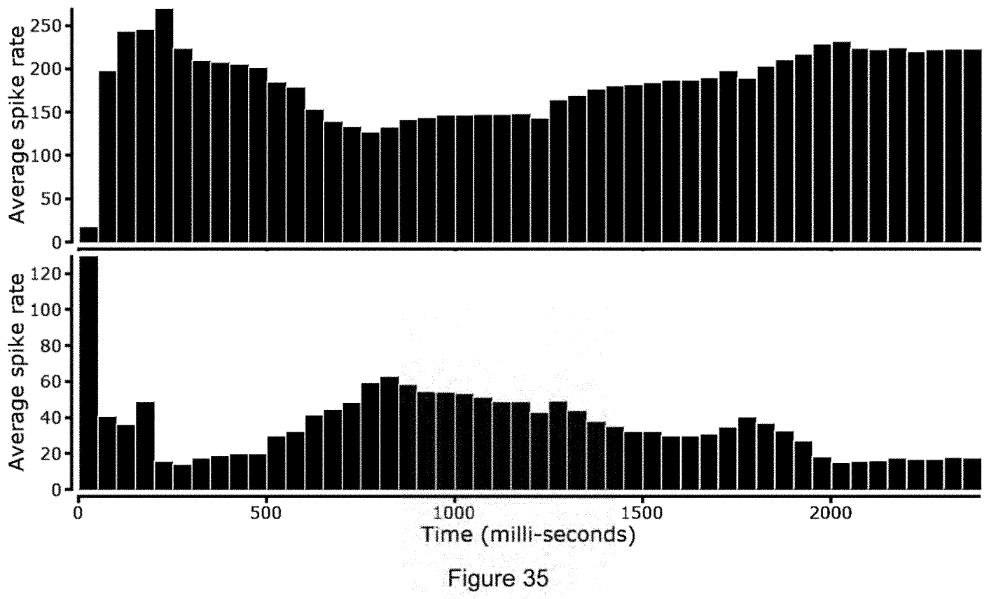

The postprocessing of the computed simulated retinal images of control (C3) contact lens design of Example 3, using the virtual retinal platform, as discussed herein, results in a spike train as a function of time (FIG. 32) and pen-stimulus histogram highlighting the average spike rate as a function of time (FIG. 33), for cells with both on- and off-polarities. The top and bottom sub-graphs of FIG. 32 and FIG. 33 represent data for on- and off-cells, respectively. The postprocessing of the computed simulated retinal images of embodiment (D3) contact lens design of Example 3, using the virtual retinal platform, as discussed herein, results in a spike train as a function of time (FIG. 34) and pen-stimulus histogram highlighting the average spike rate as a function of time (FIG. 35), for cells with both on- and off-polarities. The top and bottom sub-graphs of FIG. 34 and FIG. 35 represent data for on- and off-cells, respectively.

The neuronal activity with control (C3) contact lens, depicted as spike trains of FIG. 32, is relatively time invariant or with minimal variation or fluctuation as a function of time, for cells with both type of polarities, on- and off. On the other hand, the neuronal activity with embodiment (D1) contact lens, depicted as spike trains of FIG. 34, is relatively time variant or with greater variation or fluctuation as a function of time.

In the Example 3, the neuronal activity with control (C3) contact lens, depicted as average spike rate of FIG. 33, follows a relatively monotonous profile following the initial 100 milliseconds signifying stabilisation of the signal. This observed pattern is similar for cells with both type of polarities, on- and off. In the Example 3, the average spike rate with the control (C3) contact lens, discarding the first 100 milli-second stabilisation period, for on-type cells is approximately four times in magnitude to that obtained with off-type cells, as disclosed herein.

On the other hand, the neuronal activity with embodiment (D3) contact lens, depicted as average spike rate of FIG. 34, is time variant or non-monotonous as a function of time. In this Example 3, the cumulative average spike rate as function of time obtained with embodiment D3 is lower compared to the results obtained with control C3 of Example 3 for both on-type an off-type cells.

The non-stationarity and non-linearity in the spiking responses obtained with the embodiment lens is attributed to the artificial edges, or luminous contrast profiles, in the retinal imagery, or temporal variation of the artificial edges.

In this Example, the average spike rate as function of time, described in FIG. 28, obtained with embodiment (D2) contact lens follows a time-variant pattern for both on-type and off-type cells. While the control (C3) contact lens in this Example 3 displays some time-variance in both on-type and off-type average spike rates as depicted in FIG. 33, the temporal variation observed within the average spike rate obtained with embodiment (D3) contact lens is much greater than the control (C3) contact lens.

In this Example 3, the on- and off-axis evaluation of the optical performance, was modelled in polychromatic mode, spanning 470 nm to 650 nm, using a luminosity function describing average spectral sensitivity of human visual perception of brightness in photopic condition at 6 mm pupil analysis diameter.

In this example, the photoreceptor density as a function retinal eccentricity was kept constant for simplicity, however other variations of retinal models involving a change in photoreceptor density may be contemplated.

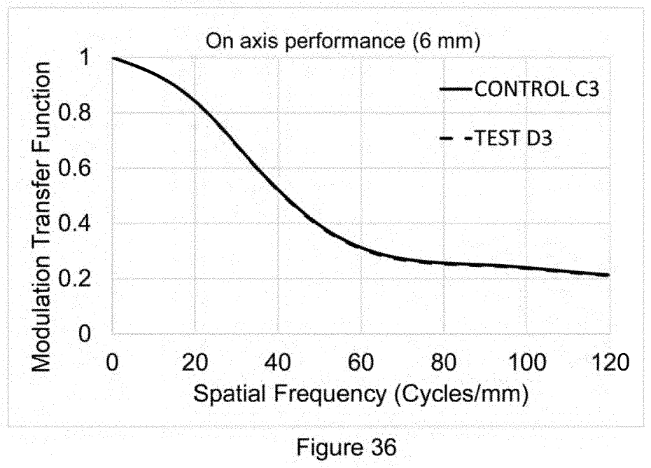

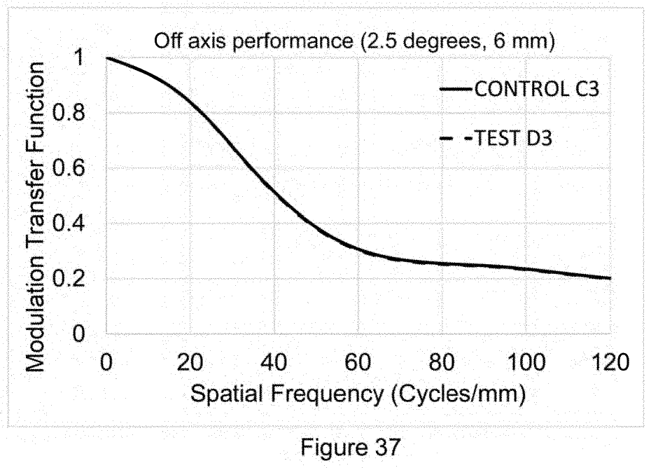

As described herein FIGS. 36 and 37, the wide-field optical performance, as gauged using modulation transfer function as function of spatial frequencies at 6 mm pupil diameter, between the control (C3) and exemplary embodiment (D3) contact lenses is virtually indistinguishable. For the off-axis performance, in Example 3, the field of view considered for evaluation of performance was 5°, which is ±2.5° from the centre.

### Example 4—Control C4 and Exemplary Embodiment Design D4

In this Example 4, the following parameters of the schematic model eye of Table 1 were altered to represent a 3 D myopic eye (i.e. base prescription Rx of −3 D) in its unaccommodated state; (i) a vitreous chamber depth of 17.65 mm, and (ii) the retinal radius of curvature to 13.5 mm. The model was configured to focus on an object approximately at optical infinity from the eye.

The modified myopic schematic model eye of was corrected, one at a time, with the control (C4) and exemplary embodiment (D4) contact lenses. The control (C4) contact lens represents a single vision lens modelled using the following parameters: front surface (R=8.262 mm, Q=−0.137), a centre thickness (0.135 mm), a back surface (R=7.75 mm, Q=−0.25) and refractive index of 1.42. The control contact lens C4 is free/devoid of any non-refractive features contemplated in this present disclosure.

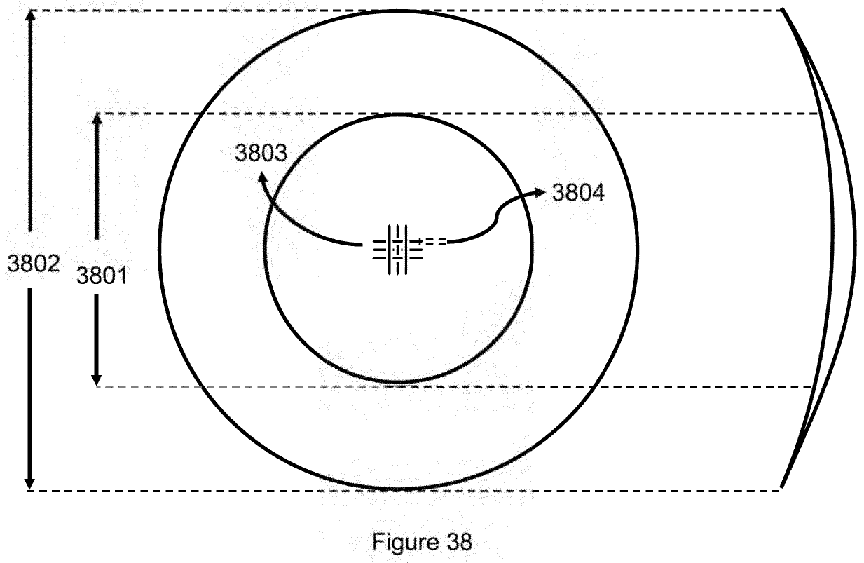

The second lens D4 represents the exemplary embodiment which is also a single vision contact lens with same parameters as control C4, that was further configured with non-refractive features disclosed in FIG. 38.

The non-refractive features of the exemplary embodiment example D4 comprises a grid pattern (3803), including a plurality of line, or striae, features. This grid pattern (3803) is positioned within the optical about the optical centre of the optic zone (3801) of the contact lens (3802). The total number of lines, or striae, -like features is 6, three in horizontal direction and three in vertical direction. The total dimension of the grid pattern is approximately 3 mm in diameter. The dimensions of each line, or striae, in the grid pattern is approximately between 75 μm×1 mm (3804). The non-refractive features are magnified relative to other features of the contact lens for identification and legibility. The remainder of the portion of the optic zone (3801) devoid of the non-refractive features of the exemplary embodiment D4 is configured with the underlying single vision prescription parameters matching the base prescription of the eye.

The simulated retinal images were computed and analysed with control C4 and embodiment D4 contact lens designs when fitted on the schematic model eye of Example 4, following the steps disclosed in paragraphs [00271] to [00273].

In this Example 4, additional variables of the virtual retinal platform were contemplated with the following settings: the option of a contrast gain control mechanism, described in Equations 1, 5 and 6, was muted. The arrangement of the neuronal bundle (1602) was in a circular arrangement spanning 15°×15° field of view. A sparse lateral connectivity mode of the virtual retina was not used.

Further, the supplementary high-pass filter option of outer plexiform layer, described in Equations 2 and 3, was muted. The post-synaptic pooling option was also muted.

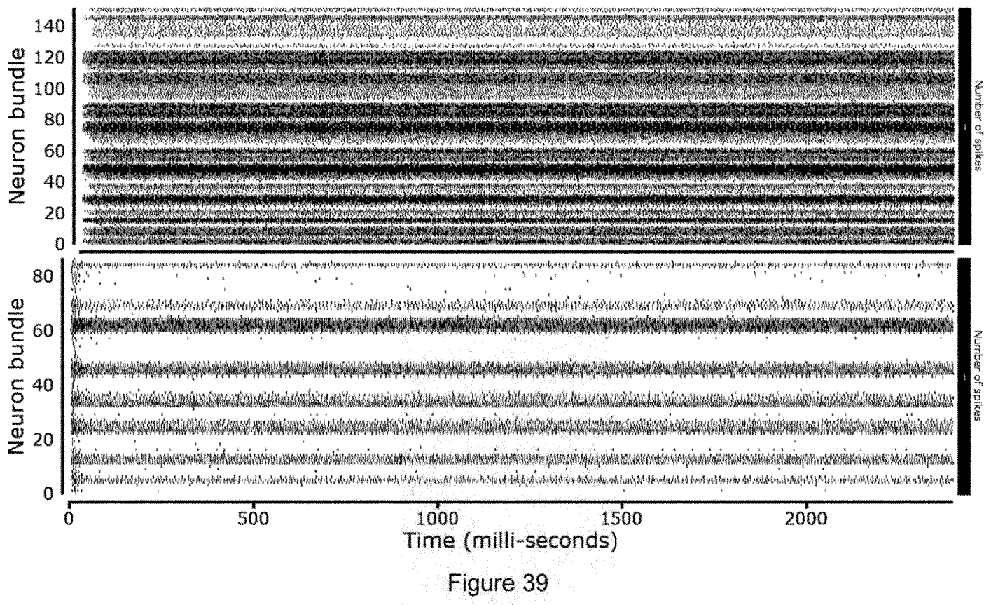

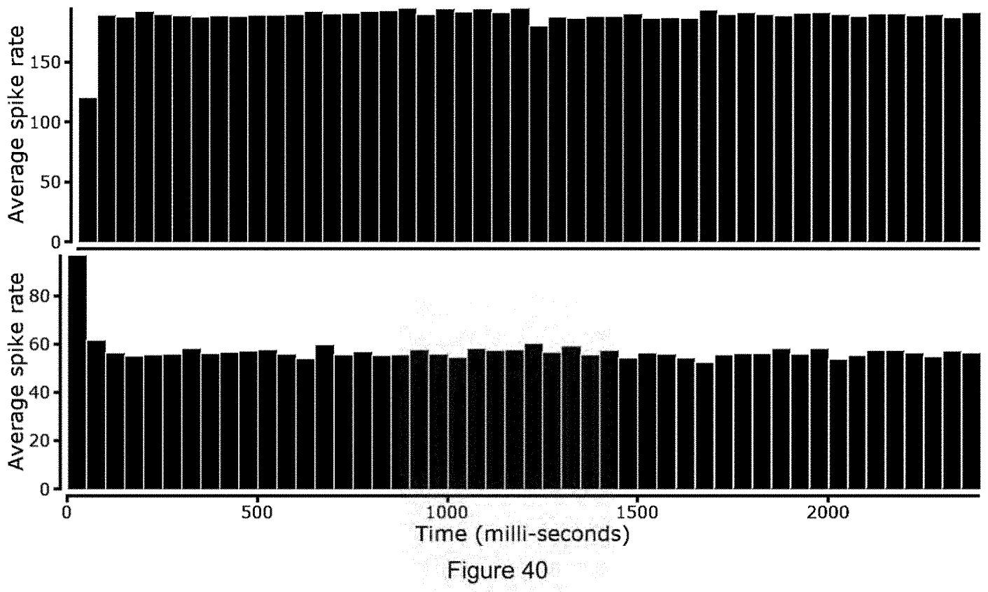

The postprocessing of the computed simulated retinal images of control (C4) contact lens design of Example 4, using the virtual retinal platform, as discussed herein, results in a spike train as a function of time (FIG. 39) and pen-stimulus histogram highlighting the average spike rate as a function of time (FIG. 40), for cells with both on- and off-polarities. The top and bottom sub-graphs of FIG. 39 and FIG. 40 represent data for on- and off-cells, respectively.

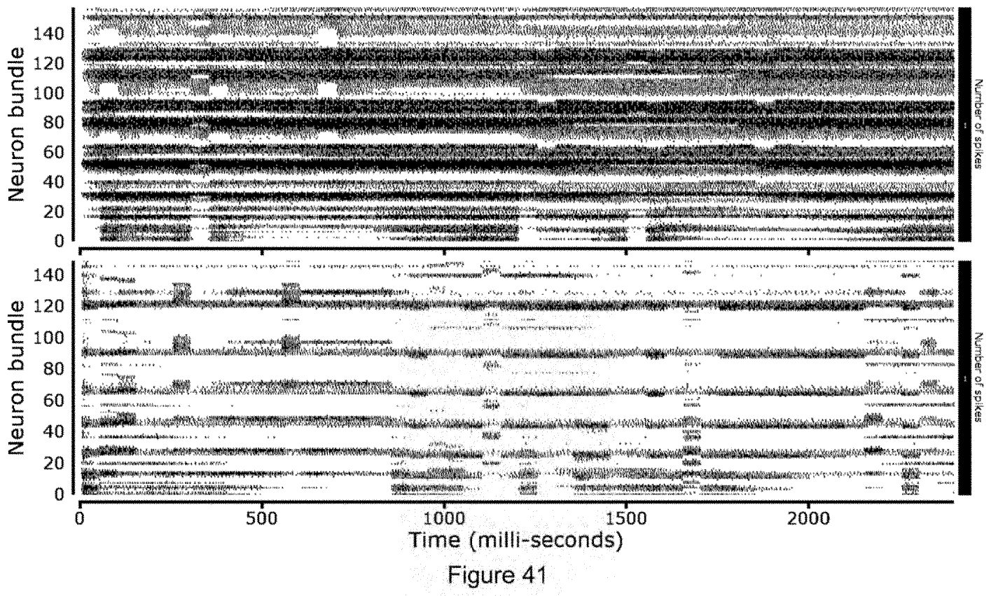

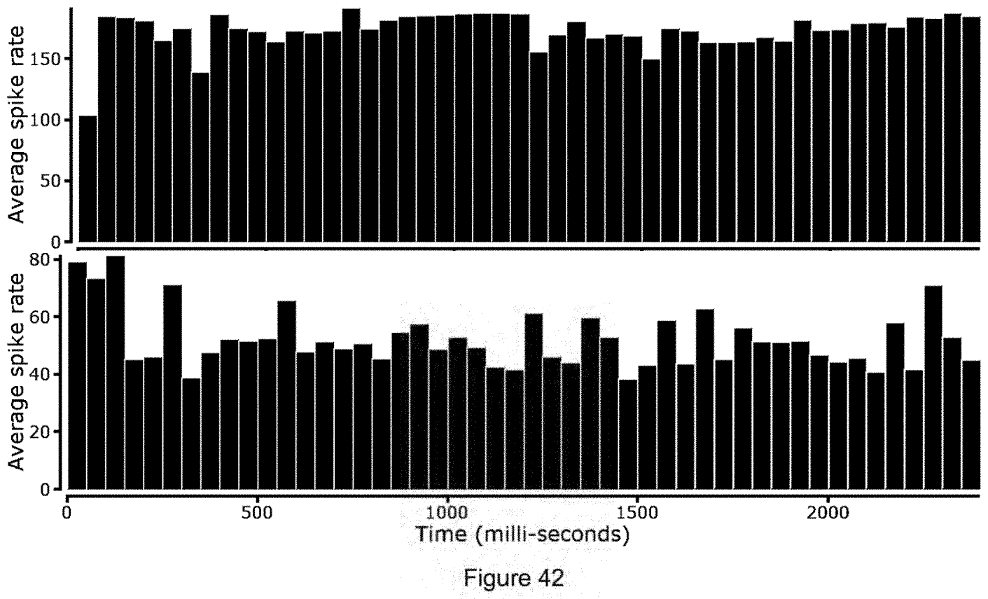

The postprocessing of the computed simulated retinal images of embodiment (D4) contact lens design of Example 4, using the virtual retinal platform, as discussed herein, results in a spike train as a function of time (FIG. 41) and peri-stimulus histogram highlighting the average spike rate as a function of time (FIG. 42), for cells with both on- and off-polarities. The top and bottom sub-graphs of FIG. 41 and FIG. 42 represent data for on- and off-cells, respectively.

The neuronal activity with control (C4) contact lens, depicted as spike trains of FIG. 39, is relatively time invariant or with minimal variation or fluctuation as a function of time, for cells with both type of polarities, on- and off. On the other hand, the neuronal activity with embodiment (D1) contact lens, depicted as spike trains of FIG. 41, is relatively time variant or with greater variation or fluctuation as a function of time.

In the Example 4, the neuronal activity with control (C4) contact lens, depicted as average spike rate of FIG. 40, follows a relatively monotonous profile following the initial 100 milliseconds signifying stabilisation of the signal. This observed pattern is similar for cells with both type of polarities, on- and off.

In the Example 4, the average spike rate with the control (C4) contact lens, discarding the first 100 milli-second stabilisation period, for on-type cells is approximately two times in magnitude to that obtained with off-type cells, as disclosed herein.

On the other hand, the neuronal activity with embodiment (D4) contact lens, depicted as average spike rate of FIG. 41, is time variant as a function of time.

In this Example, the average spike rate as function of time, described in FIG. 42, obtained with embodiment (D4) contact lens follows a time-variant for both on-type and off-type cells. The amplitude or magnitude of the temporal variation observed within the average spike rate obtained with embodiment (D4) contact lens is smaller than other embodiment contact lenses of the disclosure.

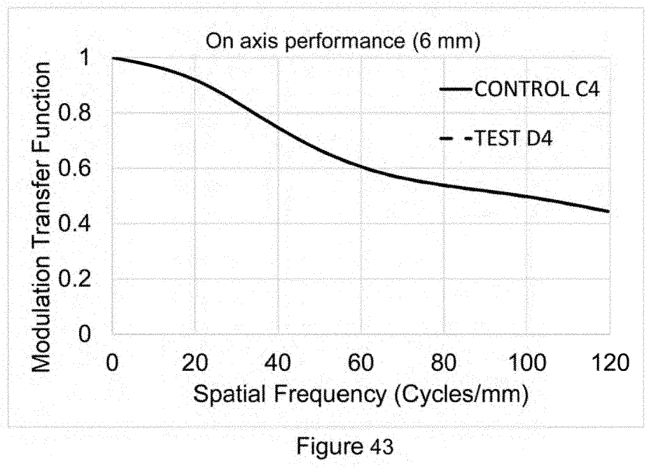

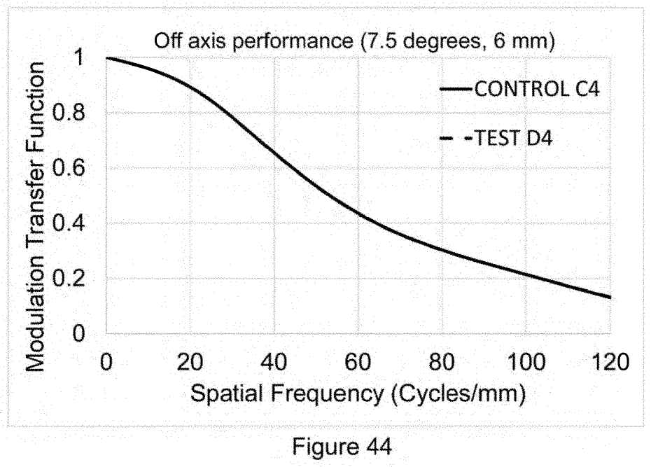

In this Example 4, the on- and off-axis evaluation of the optical performance, was modelled in monochromatic mode (589 nm) and at 4 mm pupil analysis diameter. As described herein FIGS. 43 and 44, the wide-field optical performance, as gauged using modulation transfer function as function of spatial frequencies at 6 mm pupil diameter, between the control (C4) and exemplary embodiment (D4) contact lenses is virtually indistinguishable. For the off-axis performance, in Example 4, the field of view considered for evaluation of performance was 15°, which ±7.5°.

### Example 5—Control C5 and Exemplary Embodiment Design D5

In this Example 5, the following parameters of the schematic model eye of Table 1 were altered to represent a 3D myopic eye (Rx: −3 D) in its 1 D accommodative state; (i) the vitreous chamber depth of 17.65 mm; (ii) the retinal radius of curvature to 13.5 mm; and (iii) the anterior lens radius (R=9.081 mm) and conic constant (Q=−4.123)

The model was configured to focus on a near object approximately at 1 metre from the eye. The modified myopic schematic model eye of was corrected, one at time, with the control (C5) and exemplary embodiment (D5) contact lenses.

The control (C5) contact lens represents a single vision lens modelled using the following parameters: front surface (R=8.262 mm, Q=−0.137), a centre thickness (0.135 mm), a back surface (R=7.75 mm, Q=−0.25) and refractive index of 1.42. The control contact lens C5 is free/devoid of any non-refractive features contemplated in this present disclosure.

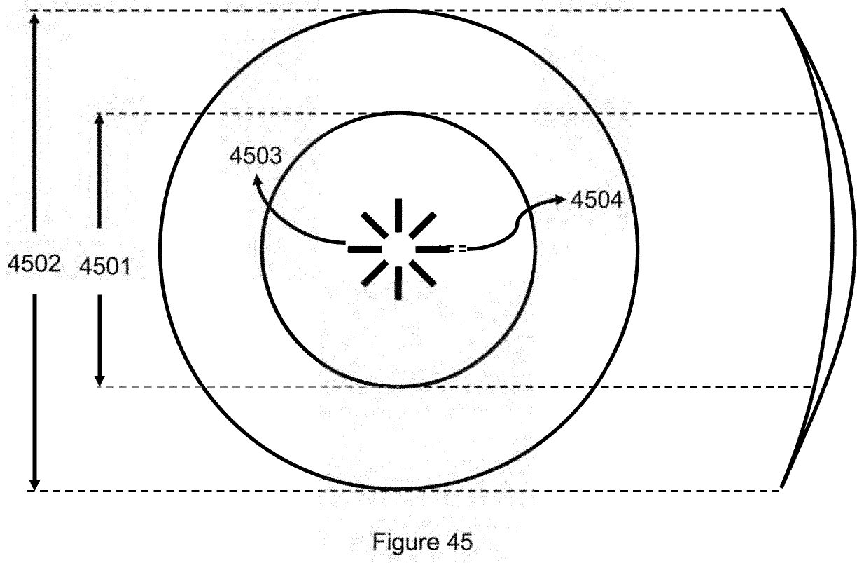

The second lens D5 represents the exemplary embodiment which is also a single vision contact lens with same parameters as control C5, that was further configured with non-refractive features disclosed in FIG. 45.

The non-refractive features of the exemplary embodiment example D5 (FIG. 45) comprises a spoke pattern (4503), including a plurality of line features. This spoke pattern (4503) is positioned within the optical zone (4501) of the contact lens (4502). The total number of spoke features is 8. The total dimension of the spoke pattern is approximately 4 mm in diameter. The dimensions of each line in the spoke pattern is approximately between 100 μm×1 mm (4504).

The non-refractive features are magnified relative to other features of the contact lens for identification and legibility. The remainder of the portion of the optic zone (4501) devoid of the non-refractive features of the exemplary embodiment D5 is configured with the underlying single vision prescription parameters matching the base prescription of the eye.

The simulated retinal images were computed and analysed with control C5 and embodiment D5 contact lens designs when fitted on the schematic model eye of Example 5, following the steps disclosed in paragraphs [00271] to [00273].

In this Example 5, additional variables of the virtual retinal platform were contemplated with the following settings; the option of a contrast gain control mechanism, described in Equations 1, 5 and 6, were used with the following input parameter values: (i) an outer plexiform amplification λ_{OPL} value of 150 Hz per normalised luminance unit; (ii) bipolar inert leaks g_{A}⁰ of 5 Hz; (iii) the feedback amplification λ_{A} of 100 in Hz; (iv) spatial scale σA of 2.5°; and (v) temporal scale τA of 0.01 milliseconds. The arrangement of the neuronal bundle (1602) was in a circular arrangement spanning 5°×5° field of view. A sparse lateral connectivity mode of the virtual retina was not used. Further, the supplementary high-pass filter option of outer plexiform layer, described in Equations 2 and 3, was muted. The post-synaptic pooling option was also muted.

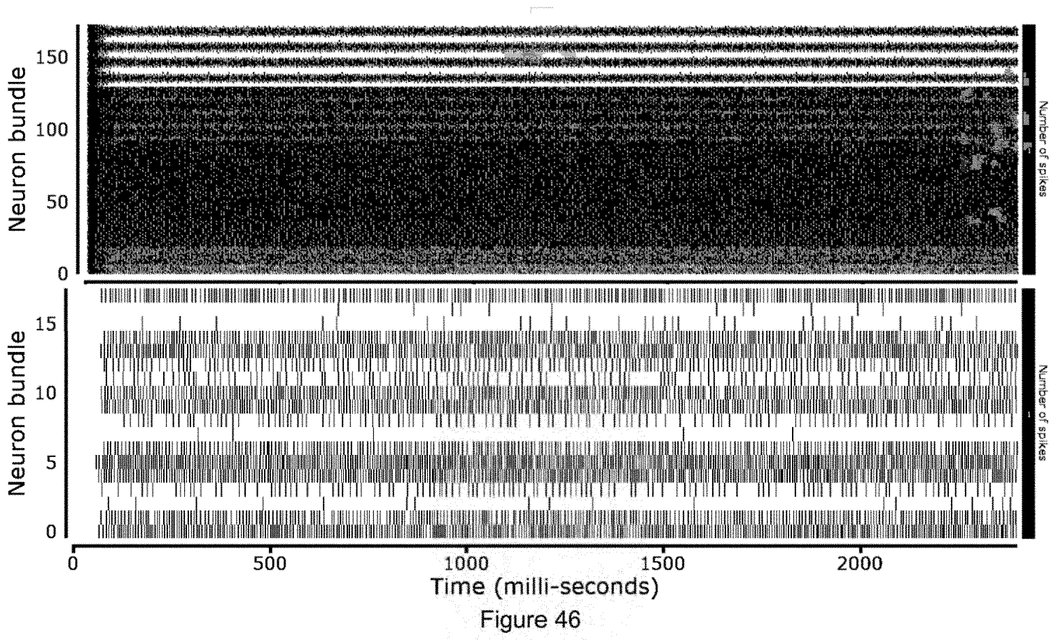

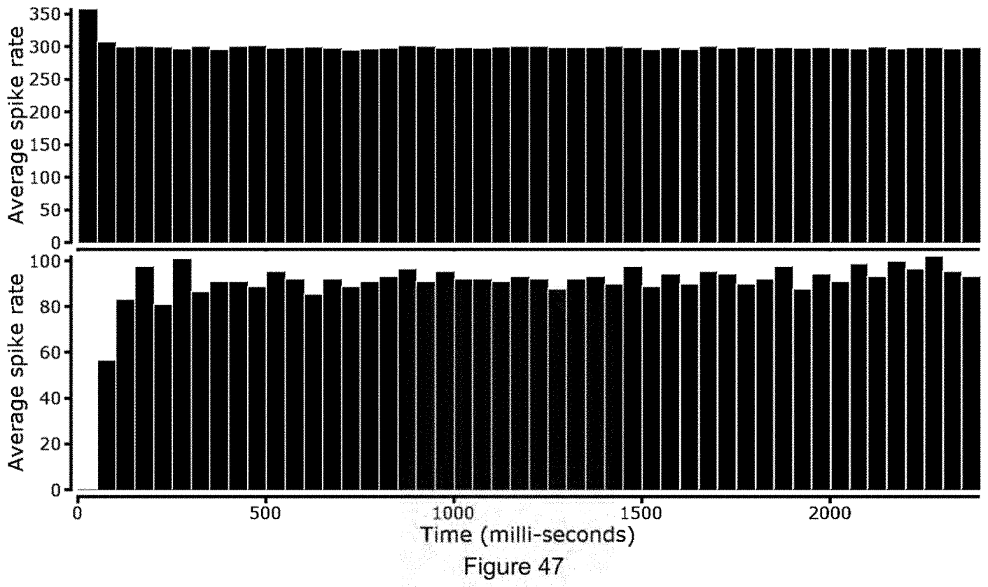

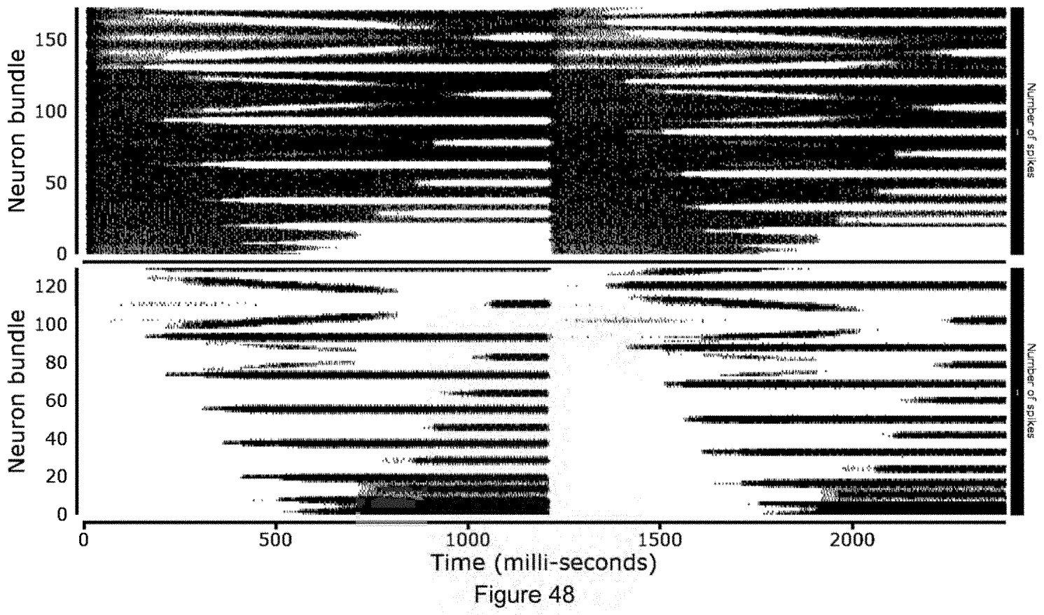

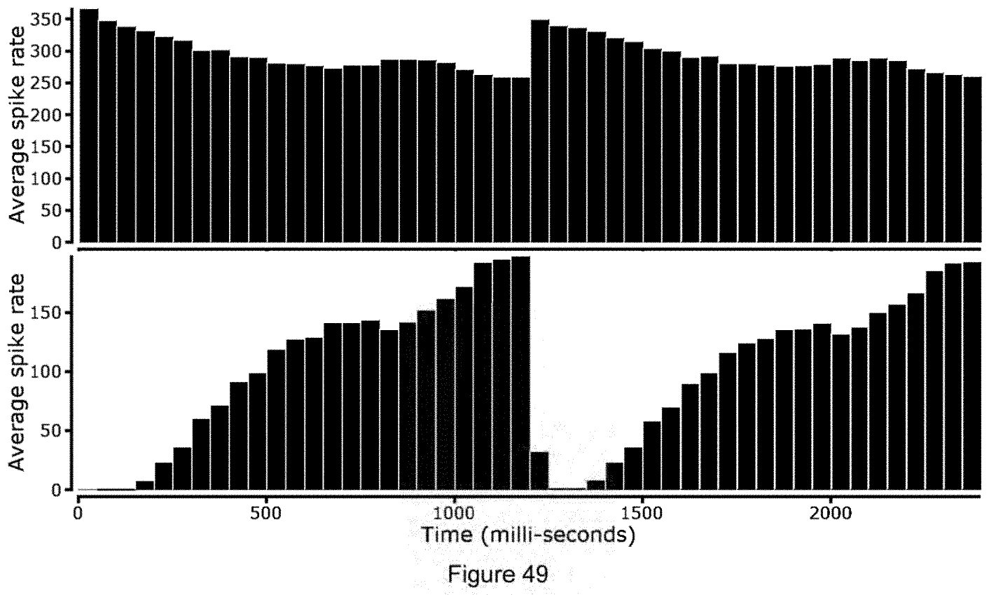

The postprocessing of the computed simulated retinal images of control (C5) contact lens design of Example 5, using the virtual retinal platform, as discussed herein, results in a spike train as a function of time (FIG. 46) and pen-stimulus histogram highlighting the average spike rate as a function of time (FIG. 47), for cells with both on- and off-polarities. The top and bottom sub-graphs of FIG. 46 and FIG. 47 represent data for on- and off-cells, respectively. The postprocessing of the computed simulated retinal images of embodiment (D5) contact lens design of Example 5, using the virtual retinal platform, as discussed herein, results in a spike train as a function of time (FIG. 48) and pen-stimulus histogram highlighting the average spike rate as a function of time (FIG. 49), for cells with both on- and off-polarities. The top and bottom sub-graphs of FIG. 48 and FIG. 49 represent data for on- and off-cells, respectively. The neuronal activity with control (C5) contact lens, depicted as spike trains of FIG. 46, is relatively time invariant or with minimal variation or fluctuation as a function of time, for cells with both type of polarities, on and off. On the other hand, the neuronal activity with embodiment (D5) contact lens, depicted as spike trains of FIG. 48, is relatively time variant and monotonically decreasing or increasing as a function of time.

In the Example 5, the neuronal activity with control (C5) contact lens, depicted as average spike rate of FIG. 47, follows a relatively monotonous profile following the initial 100 milliseconds signifying stabilisation of the signal. This observed pattern is similar for cells with both type of polarities, on and off. In the Example 5, the average spike rate with control (C5) contact lenses, discarding the first 100 millisecond stabilisation period, for on-type cells is approximately three times in magnitude to that obtained with off-type cells, as disclosed herein.

On the other hand, the neuronal activity with embodiment (D5) contact lens, depicted as average spike rate of FIG. 49, is time variant as a function of time. In this Example, the average spike rate as function of time, described in FIG. 49, obtained with embodiment (D5) contact lens follows a time-variant for both on-type and off-type cells. The non-stationarity and non-linearity in the spiking responses obtained with the embodiment lens is attributed to the artificial edges, or luminous contrast profiles, in the retinal imagery, or temporal variation of the artificial edges.

In this Example 5, the on- and off-axis evaluation of the optical performance, was modelled in polychromatic mode using a luminosity function describing average spectral sensitivity of human visual perception of brightness in photopic condition at 5 mm pupil analysis diameter.

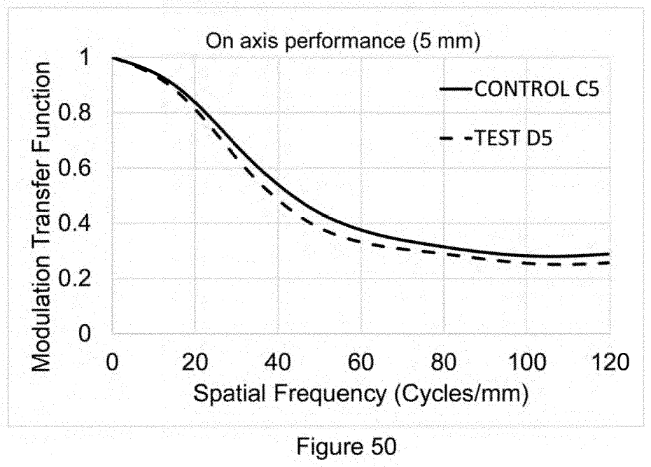

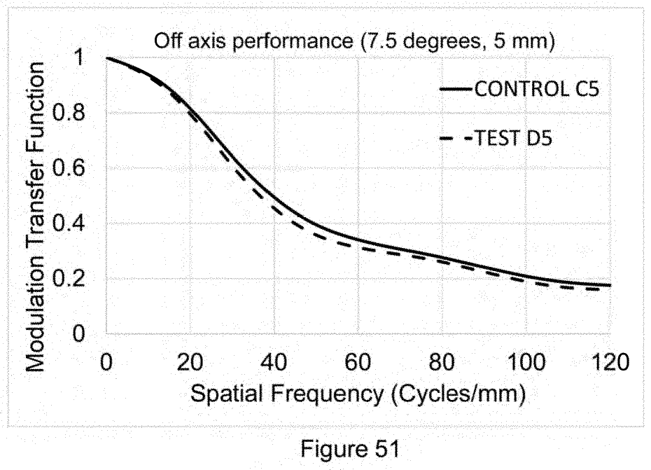

As described herein FIGS. 50 and 51, the wide-field optical performance, as gauged using modulation transfer function as function of spatial frequencies at 5 mm pupil diameter, between the control (C5) and exemplary embodiment (D5) contact lenses is significantly similar, i.e. with less than 5% variation in the area under the curves represented by the solid black line and dashed black lines. For the off-axis performance, in Example 5, the field of view considered for evaluation of performance was 15°, which ±7.5°.

### Example 6—Control C6 and Exemplary Embodiment Design D6

In this Example 6, the following parameters of the schematic model eye of Table 1 were altered to represent a 4 D myopic eye (i.e. base prescription Rx of −4 D) in its 2 D accommodated state; (i) vitreous chamber depth of 18.04 mm; (ii) retinal radius of curvature of 13.5 mm; and (iii) anterior lens radius (R=7.794 mm) and conic constant (Q=−3.959) parameters.

The model was configured to focus on a near object approximately at 50 cm from the eye. The modified myopic schematic model eye of was corrected, one at time, with the control (C6) and exemplary embodiment (D6) contact lenses. The control (C6) contact lens represents a single vision lens modelled using the following parameters: front surface (R=8.41 mm, Q=−0.112), a centre thickness (0.135 mm), a back surface (R=7.75 mm, Q=−0.25) and refractive index of 1.42. The control contact lens C6 is free/devoid of any non-refractive features contemplated in this present disclosure.

The second lens D6 represents the exemplary embodiment which is also a single vision contact lens with same parameters as control C6, that was further configured with non-refractive features disclosed in FIG. 45.

The non-refractive features of the exemplary embodiment example D6 comprises a random pattern (5203), including a plurality of elliptical dot-like features, marginally elongated in the horizontal direction. This random pattern is positioned within the optical zone (5201) about the optical centre of the exemplary embodiment contact lens (5202). The total number of elliptical dot-like features in (5202) is 18. The total dimension of the random pattern is approximately 3 mm in diameter. The dimensions of each elliptical dot-like feature is approximately between 125 μm×200 μm (5204).

The non-refractive features are magnified relative to other features of the contact lens for identification and legibility. The remainder of the portion of the optic zone (5201) devoid of the non-refractive features of the exemplary embodiment D8 is configured with the underlying single vision prescription parameters matching the base prescription of the eye.

The simulated retinal images were computed and analysed with control C6 and embodiment D6 contact lens designs when fitted on the schematic model eye of Example 6, following the steps disclosed in paragraphs [00271] to [00273]. In this Example 6, additional variables of the virtual retinal platform were contemplated with the following settings: the option of a contrast gain control mechanism, described in Equations 1, 5 and 6, was muted. The arrangement of the neuronal bundle (1602) was in a circular arrangement spanning 15°×15° field of view.

A sparse lateral connectivity mode of the virtual retina was used with 10 pre-synaptic neurons with 10% of positive weight and a weight variance of 0.01. The supplementary high-pass filter option of outer plexiform layer, described in Equations 2 and 3, was muted. The post-synaptic pooling option was also muted.

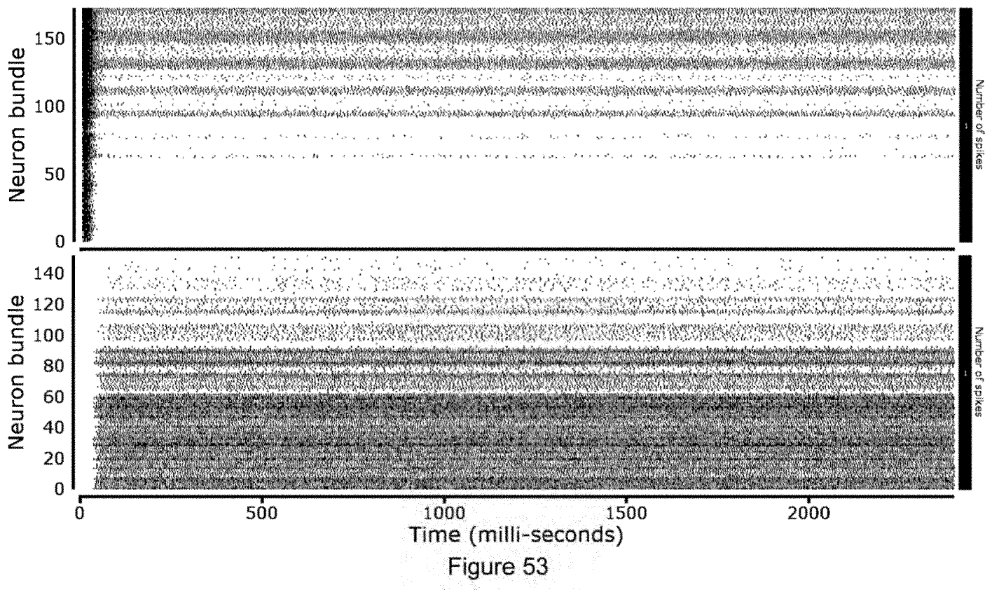

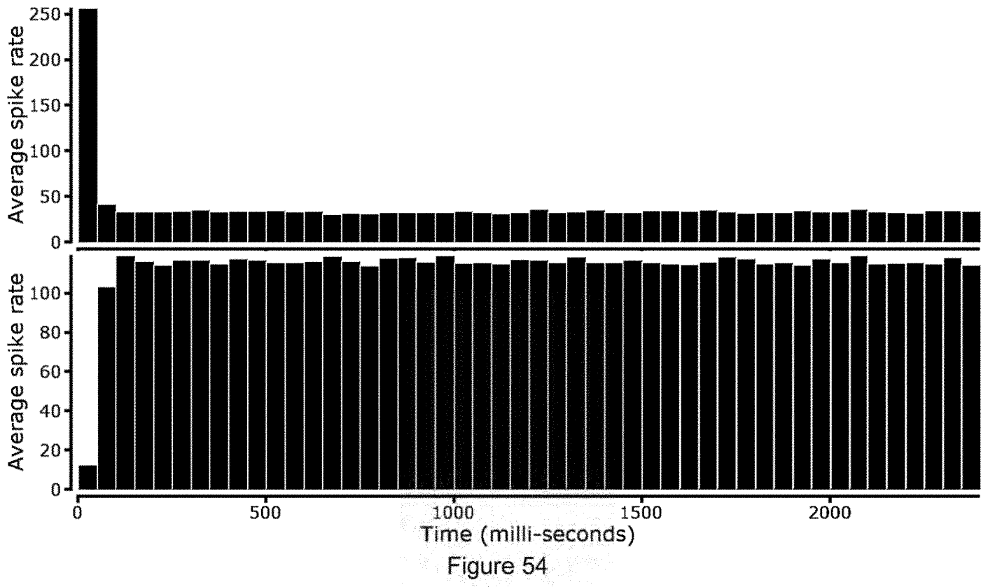

The postprocessing of the computed simulated retinal images of control (C6) contact lens design of Example 6, using the virtual retinal platform, as discussed herein, results in a spike train as a function of time (FIG. 53) and pen-stimulus histogram highlighting the average spike rate as a function of time (FIG. 54), for cells with both on- and off-polarities. The top and bottom sub-graphs of FIG. 53 and FIG. 54 represent data for on- and off-cells, respectively.

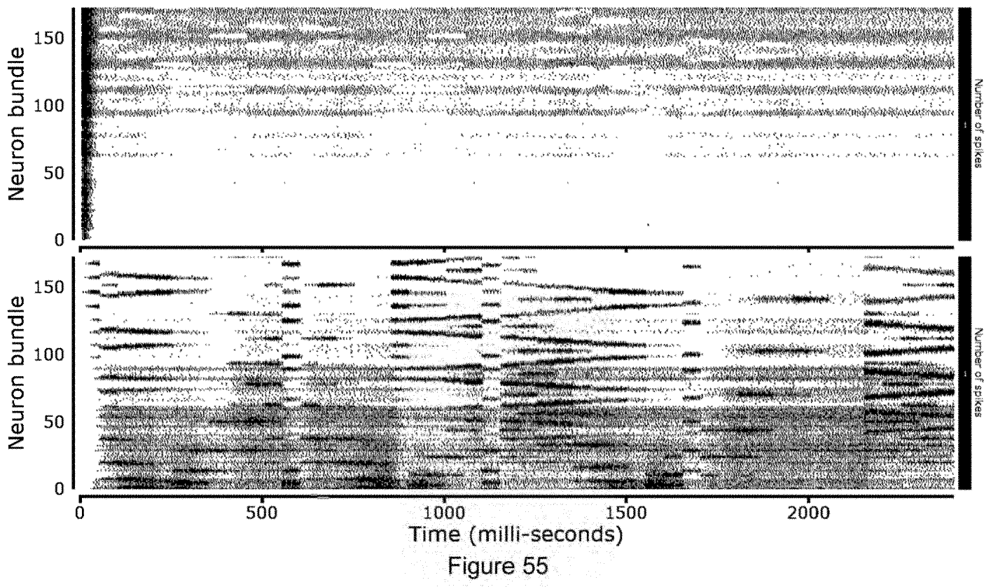

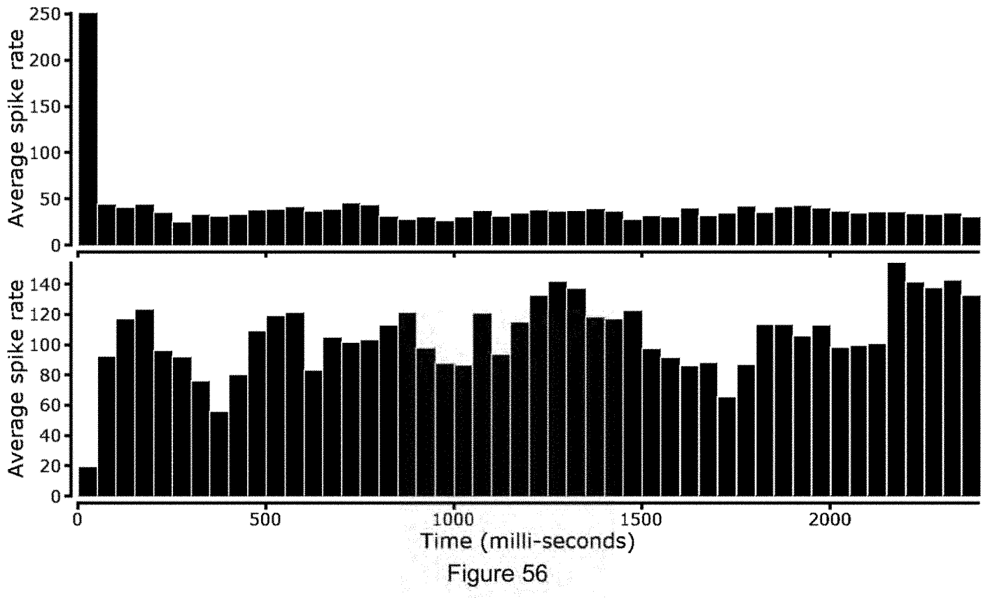

The postprocessing of the computed simulated retinal images of embodiment (D6) contact lens design of Example 6, using the virtual retinal platform, as discussed herein, results in a spike train as a function of time (FIG. 55) and peri-stimulus histogram highlighting the average spike rate as a function of time (FIG. 56 for cells with both on- and off-polarities. The top and bottom sub-graphs of FIG. 55 and FIG. 56 represent data for on- and off-cells, respectively.

The neuronal activity with control (C6) contact lens, depicted as spike trains of FIG. 53, is relatively time invariant or with minimal variation or fluctuation as a function of time, for cells with both type of polarities, on- and off. On the other hand, the neuronal activity with embodiment (D6) contact lens, depicted as spike trains of FIG. 55, is relatively time variant and monotonically decreasing or increasing as a function of time. The non-stationarity and non-linearity in the spiking responses obtained with the embodiment lens is attributed to the artificial edges, or luminous contrast profiles, in the retinal imagery, or temporal variation of the artificial edges.

In the Example 6, the neuronal activity with control (C6) contact lens, depicted as average spike rate of FIG. 54, follows a relatively monotonous profile following the initial 100 milliseconds signifying stabilisation of the signal. This observed pattern is similar for cells with both type of polarities, on- and off. In the Example 6, the average spike rate with the control (C6) contact lens, discarding the first 100 milli-second stabilisation period, for on-type cells is approximately three times in magnitude to that obtained with off-type cells, as disclosed herein.

On the other hand, the neuronal activity with embodiment (D6) contact lens, depicted as average spike rate of FIG. 56, is time variant as a function of time. In this Example, the average spike rate as function of time, described in FIG. 56, obtained with embodiment (D6) contact lens follows a time-variant pattern for both on-type and off-type cells.

In this Example 6, the on- and off-axis evaluation of the optical performance, was modelled in monochromatic mode (589 nm) and at 4 mm pupil analysis diameter.

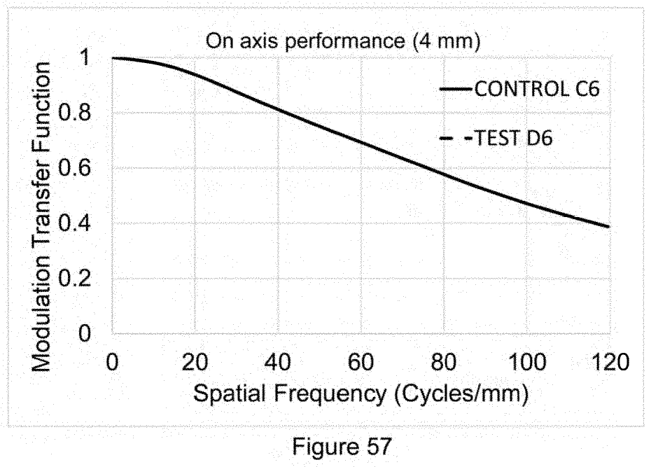

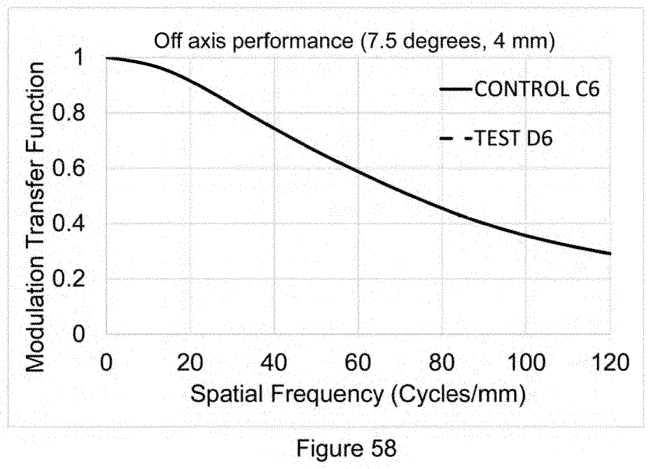

As described herein FIGS. 57 and 58, the wide-field optical performance, as gauged using modulation transfer function as function of spatial frequencies at 4 mm pupil diameter, between the control (C6) and exemplary embodiment (D6) contact lenses are virtually indistinguishable represented by the solid black line and dashed black lines. For the off-axis performance, in Example 6, the field of view considered for evaluation of performance was 15°, which ±7.5°.

### Example 7—Control C7 and Exemplary Embodiment Design D7

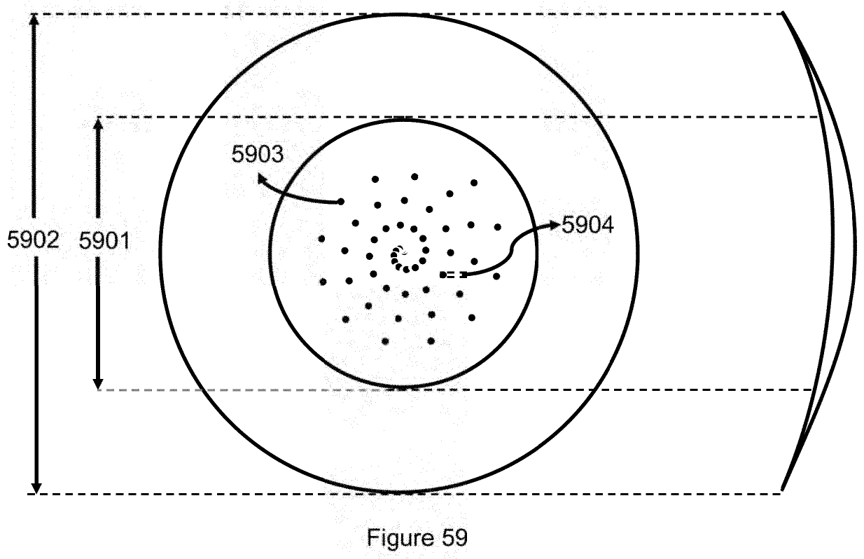

In this Example 7, the following parameters of the schematic model eye of Table 1 were altered to represent a 4 D myopic eye (i.e. a base prescription Rx of −4 D) in its unaccommodated state: (i) a vitreous chamber depth of the eye of 18.04 mm, and (ii) retinal radius of curvature to 13.5 mm. The model was configured to focus on a distant object at optical infinity from the eye. The modified myopic schematic model eye of was corrected, one at time, with the control (C7) and exemplary embodiment (D7) contact lenses. The control (C7) contact lens represents a single vision lens modelled using the following parameters: front surface (R=8.41 mm, Q=−0.112), a centre thickness (0.135 mm), a back surface (R=7.75 mm, Q=−0.25) and refractive index of 1.42. The control contact lens C7 is free/devoid of any non-refractive features contemplated in this present disclosure. The second lens D7 represents the exemplary embodiment which is also a single vision contact lens with same parameters as control C7, that was further configured with non-refractive features disclosed in FIG. 59.

The non-refractive features of the exemplary embodiment example D7 comprises a spiral pattern (5903), including a plurality of dot-like features. The spiral pattern is positioned within the optical zone (5901) of the contact lens (5902). The total number of dot-like features in each arm is 49. The total dimension of the spiral pattern is approximately 6 mm in diameter. The width of each dot-like feature is approximately between 125 μm (5904). The non-refractive features are magnified relative to other features of the contact lens for identification and legibility. The remainder of the portion of the optic zone (5901) devoid of the non-refractive features of the exemplary embodiment D7 is configured with the underlying single vision prescription parameters matching the base prescription of the eye.

The simulated retinal images were computed and analysed with control C7 and embodiment D7 contact lens designs when fitted on the schematic model eye of Example 7, following the steps disclosed in paragraphs [00271] to [00273].

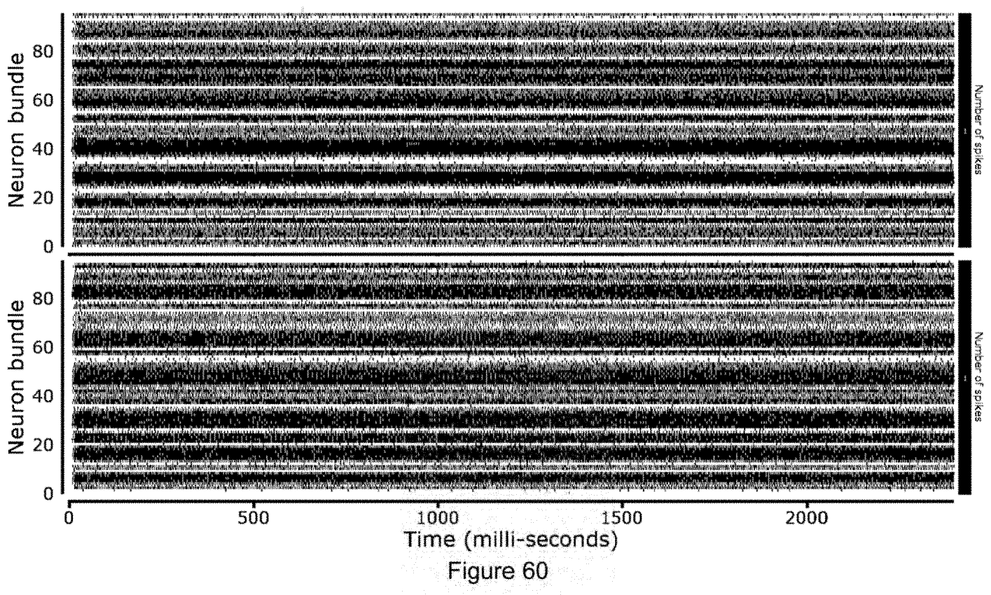

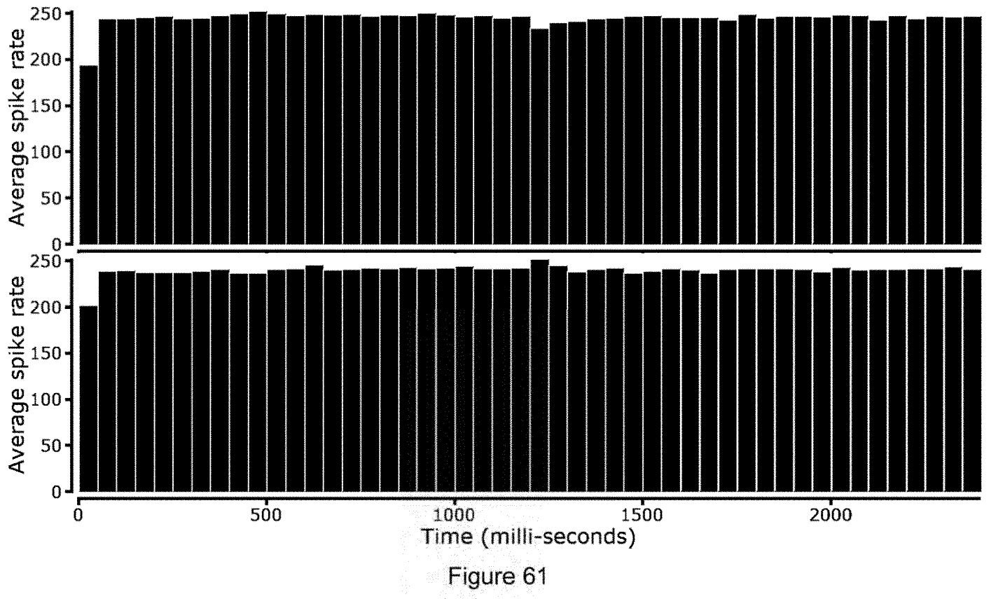

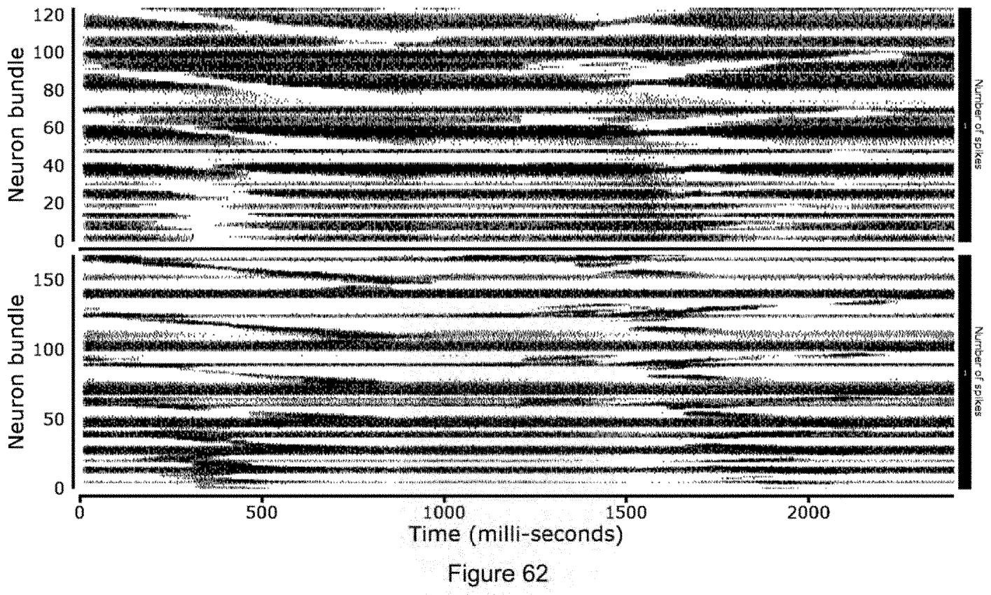

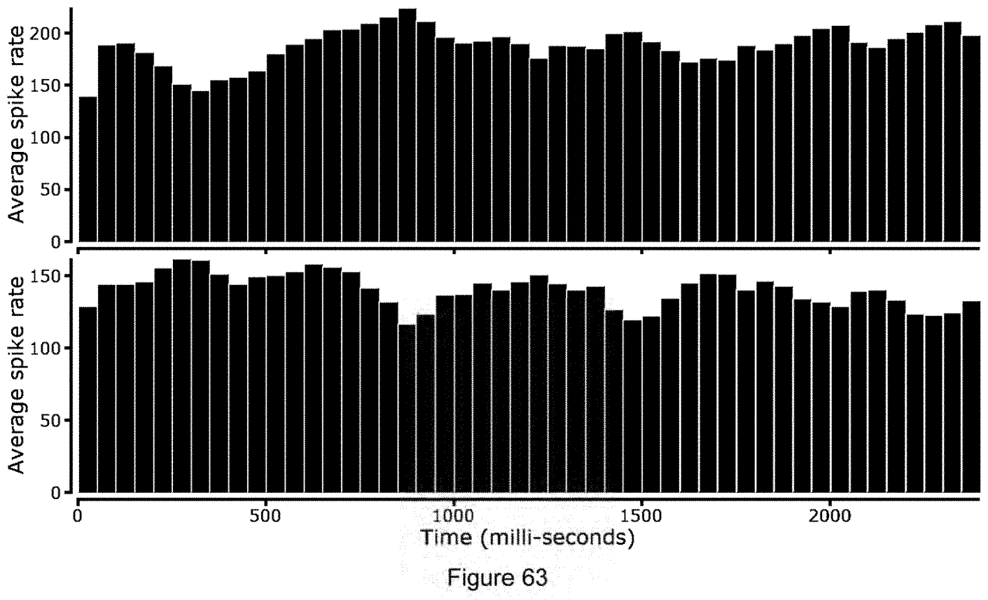

In this Example 7, additional variables of the virtual retinal platform were contemplated with the following settings; the option of a contrast gain control mechanism, described in Equations 1, 5 and 6, were used with the following input parameter values: (i) an outer plexiform amplification λ_{OPL} value of 150 Hz per normalised luminance unit; (ii) bipolar inert leaks g_{A}⁰ of 5 Hz; (iii) the feedback amplification λ_{A} of 100 in Hz; (iv) spatial scale σA of 2.5°; and (v) temporal scale τA of 0.01 milli-seconds. The arrangement of the neuronal bundle (1602) was in a circular arrangement spanning 5°×5° field of view. A sparse lateral connectivity mode of the virtual retina was muted. The supplementary high-pass filter option of outer plexiform layer, described in Equations 2 and 3, was muted. The post-synaptic pooling option was also muted. The postprocessing of the computed simulated retinal images of control (C7) contact lens design of Example 7, using the virtual retinal platform, as discussed herein, results in a spike train as a function of time (FIG. 60) and peri-stimulus histogram highlighting the average spike rate as a function of time (FIG. 61), for cells with both on- and off-polarities. The top and bottom sub-graphs of FIG. 60 and FIG. 61 represent data for on- and off-cells, respectively. The postprocessing of the computed simulated retinal images of embodiment (D7) contact lens design of Example 7, using the virtual retinal platform, as discussed herein, results in a spike train as a function of time (FIG. 62) and pen-stimulus histogram highlighting the average spike rate as a function of time (FIG. 63 for cells with both on- and off-polarities. The top and bottom sub-graphs of FIG. 62 and FIG. 63 represent data for on- and off-cells, respectively.

The neuronal activity with control (C7) contact lens, depicted as spike trains of FIG. 60, is relatively time invariant or with minimal variation or fluctuation as a function of time, for cells with both type of polarities, on- and off. On the other hand, the neuronal activity with embodiment (D7) contact lens, depicted as spike trains of FIG. 62, is relatively time variant and fluctuating with varying periodicity as a function of time.

In the Example 7, the neuronal activity with control (C7) contact lens, depicted as average spike rate of FIG. 61, follows a relatively monotonous profile following the initial 100 milliseconds, signifying stabilisation of the signal. This observed pattern is similar for cells with both type of polarities, on- and off.

On the other hand, the neuronal activity with embodiment (D6) contact lens, depicted as average spike rate of FIG. 62, is variant as a function of time. In this Example, the average spike rate as function of time, described in FIG. 63, obtained with embodiment (D7) contact lens follows a time-variant pattern for both on-type and off-type cells.

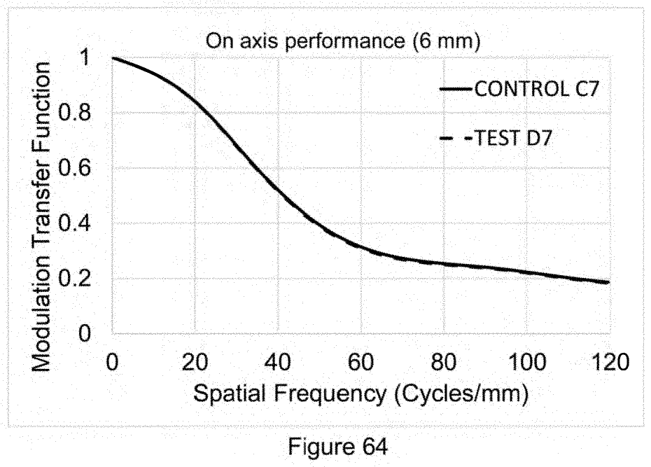

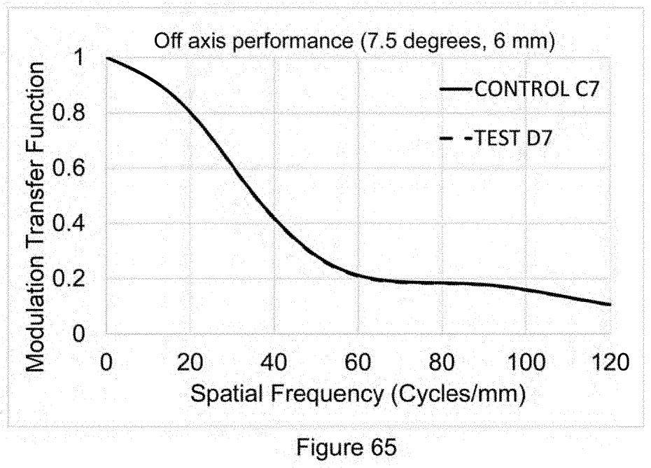

In this Example 7, the on- and off-axis evaluation of the optical performance, was modelled in monochromatic mode (589 nm) and at 6 mm pupil analysis diameter. As described herein FIGS. 64 and 65, the wide-field optical performance, as gauged using modulation transfer function as function of spatial frequencies at 6 mm pupil diameter, between the control (C7) and exemplary embodiment (D7) contact lenses are virtually indistinguishable represented by the solid black line and dashed black lines.

For the off-axis performance, in Example 7, the field of view considered for evaluation of performance was 15°, which ±7.5°. The simulation techniques described herein are one of many methods to demonstrate that the single vision contact lenses with contemplated non-refractive features disclosed herein provide an increase in the retinal ganglion cell activity compared to the standard of care single vision contact lens.

### Spectacle Lens Embodiments

Various spectacle lens embodiments are modelled to demonstrate that the non-refractive features, used in conjunction with the single vision optical profiles, provides an increase in the retinal ganglion cell activity, gauged by the surrogate measures of an increase in the average retinal ganglion cell spike rate with the virtual retinal platform, emulating the performance in the eye of the wearer.

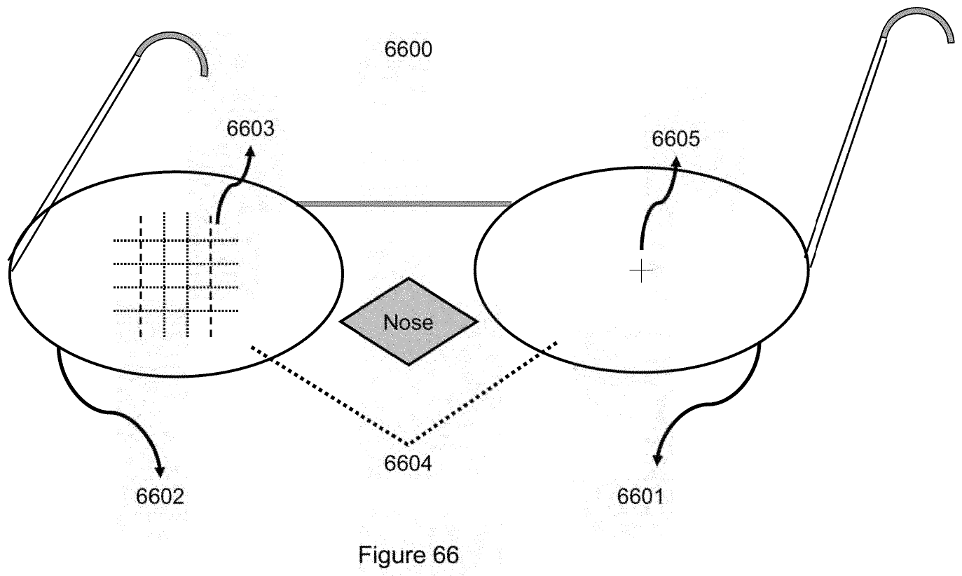

FIG. 66 shows the frontal view of a spectacle lens of prior art (6601) and an exemplary spectacle lens (6602) embodiment, not to scale. The dimensions of the spectacle lenses are approximately 40 mm×50 mm. In both cases, the entire spectacle lens area constitutes its optic zone. The spectacle lens embodiment (6602) is configured with non-refractive feature (6603) comprising a grid pattern, including 4 horizontal lines, or striae and 4 vertical lines, or striae. The optic zone designed substantially about the optic centre (6605) with single vision refractive power matching the base prescription of the eye. The grid pattern positioned in the centre of the spectacle lens embodiment spanning about 25 mm in height and width. The borders of these grid lines (6603) configured completely opaque or substantially opaque. The width of the non-refractive features (6604) is approximately between 50 μm and 100 μm, it is only magnified in the figure to demonstrate the feature relative to the size of the contact lens described herein. The embodiment of FIG. 66 may also be configured in other variations, for example, the width of contemplated non-refractive design features within the optic zone may be at least 125 μm, 150 μm, 175 μm, 200 μm, or 250 μm, in width. The embodiment of FIG. 66 may also be configured in other variations, for example, the contemplated non-refractive design features may include a random pattern, a plurality of circular, elliptical, triangular, rectangular, hexagonal, regular polygon or irregular polygons; wherein the width of the border defining the plurality of apertures may be between 50 and 125 μm, 150 and 250 μm, or 100 and 300 μm in width. In a preferred variation of the embodiment of FIG. 66, the maximum width of the non-refractive features, i.e. width of the lines forming a grid pattern, or any other pattern, does not to exceed 150 μm, 200 μm or 250 μm to avoid unwarranted consequential effects on the resolution characteristics of the wearer's eye. In other embodiments, the contemplated non-refractive design feature may be positioned in the located in periphery of the optic zone of the spectacle design. In yet another spectacle lens embodiment, the number of fine lines, or striae, forming the grid pattern, may be at least 5, 9, 15 or 25. In some other spectacle lens embodiment, the number of design features, lines or striae, forming the grid pattern, may be between 5 and 9, or between 5 and 15, or between 9 and 15, or between 5 and 25. In one another embodiment, only one long substantially unbroken curvilinear line or a zig-zag line may be contemplated to run through the optical zone with a length of at least 3 mm, 6 mm, 9 mm, or 12 mm.

In yet another spectacle lens embodiment, the one or more striations may be arranged in a symmetric or random fashion, they may be positioned concentric with the optical axis or decentred with respect to the optical centre. Striations may also consist of straight or curved lines, they may touch or cross each other, or all be configured in isolation, or a combination thereof. Striations may vary in width and length. There may be different patterns applied to lenses worn in the left and right eye.

In yet another spectacle lens embodiment the contemplated design features (i.e. a plurality of striations or moiré pattern) within the spectacle lens may be separated from each other. In yet another embodiment, the contemplated plurality of non-refractive features may be configured adjacent to or interlaced with each other.

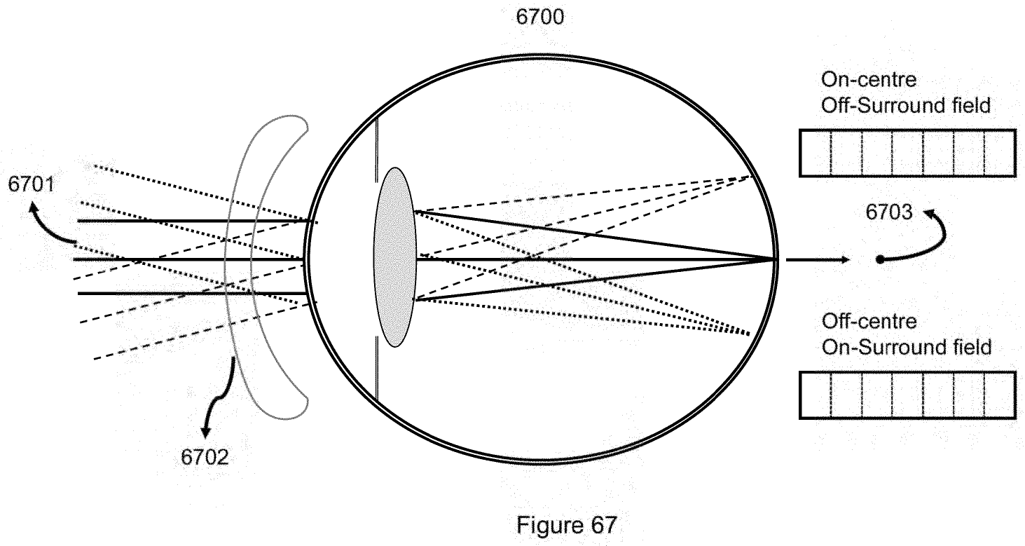

The natural saccadic eye movements the spectacle wearer may result in a temporally varying stimulus, which may further boost the inhomogeneity artificially introduced into the visual imagery, which may in-turn boost the potency of the therapeutic benefits for the wearer, for example, a greater reduction in the rate of myopia progression for a wearer. FIG. 67 shows the schematic diagram depicting incoming light, of a visible wavelength, for example, 555 nm, with a vergence 0 D, from a wide-angle field of view (6701) entering a 2 D myopic model eye (6700), which is corrected with a standard single vision lens of prior art (6702). The retinal ganglion cell activity recorded by the on-centre/off-surround and off-centre/on-surround circuitry (6703). The retinal ganglion cell activity with the standard single vision lens of prior art (6702) was captured at the retinal plane facilitated by simulated habitual saccadic eye movements, demonstrating minimal retinal activity or retinal activity at a basal rate or minimal temporal variation of the retinal activity. The relative differences in the temporal integration of on- and off-receptive field activity determines further eye growth.

The present disclosure postulates that an inactive retina triggers eye growth and an active retina reduces the growth or triggers a stop signal. The present disclosure further contemplates that a standard single vision or a spectacle lens of the prior art and/or a spatially homogenous visual imagery contributes to a homogeneous and substantially edge-less visual imagery leaving the retina in a baseline state (i.e. baseline or constant firing pattern of the retinal ganglion cells) and thus promoting further eye growth leading to more myopia.

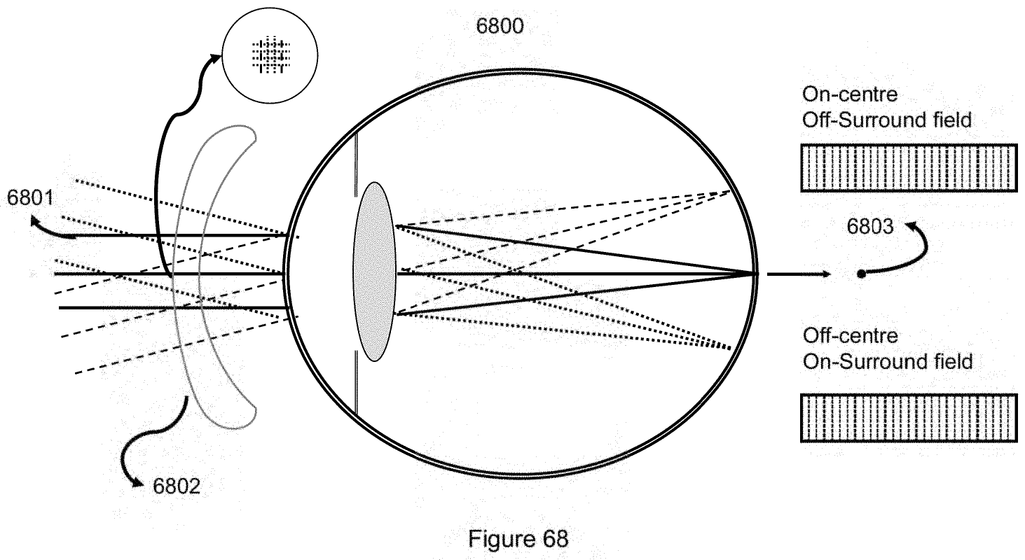

FIG. 68 shows the schematic diagram depicting incoming light, of a visible wavelength, for example, 555 nm, with a vergence 0 D, from a wide-angle field of view (6801) entering a 2 D myopic model eye (6800), which is corrected with a spectacle embodiment (6802). The retinal ganglion cell activity recorded by the on-centre/off-surround and off-centre/on-surround circuitry (6803) with the standard spectacle embodiment (6802) was captured at the retinal plane facilitated by simulated habitual saccadic eye movements, demonstrating or showing increased activity at the retina compared to the baseline state.

A simple model eye was chosen for illustrative purposes in FIGS. 67 and 68, However, in other embodiments, schematic raytracing model eyes like Liou-Brennan, Escudero-Navarro and others may be used instead. The examples provided herein have used a 2 D myopic model eye to disclose the present disclosure, however the same disclosure can be extended to other degrees of myopia, i.e. −1 D, −3 D, −5 D or −6 D. Further, it is understood that one can draw extensions to eyes with varying degrees of myopia in conjunction with astigmatism. In the embodiments, reference was made to a specific wavelength of 555 nm, however it is understood that one can draw extensions to other visible wavelengths between 420 nm and 760 nm.

Modelling of various exemplary spectacle lens embodiments (D8 to D10) demonstrate that the contemplated non-refractive features used in conjunction with the single vision designs provide an increase in the retinal ganglion cell activity, gauged by the increase in the average retinal spike rate obtained using the virtual retinal platform disclosed herein. In other embodiments, various other surrogate measures of retinal ganglion cell activity may be considered, for example, inspection of the spike train analysis for the neuronal bundle of choice.

To demonstrate the workings for the spectacle lens embodiments in accordance with the disclosure, advanced optical modelling experiments were conducted using two different types of spectacle for each test case (i.e. Examples 8 to 10) as described herein.

The first type included single vision spectacle lenses (C8 to C10) that were matched to the base prescription of the schematic model eyes, to provide correction of the refractive error, emulating the standard of care.

The second type included various exemplary spectacle lens embodiments (D8 to D10), which are essentially the same single vision, standard of care, control spectacle lenses (C8 to C10) which are further configured with additional non-refractive features, designed in accordance with the invention. To demonstrate the workings of the invention, the control (C8 to C10) and exemplary embodiment spectacle lenses (D8 to D10) were fitted, tested/evaluated, one at a time, on the modified schematic model eyes described in Examples 8 to 10, respectively. The surface transmission properties of the front surface of the spectacle lens was modified to design the features of Examples 8 to 10. The transmission is computed as a fraction of 100%, with 100% meaning that all of the light is transmitted as if there were no absorption, reflection, or vignetting losses. In certain embodiments of the present disclosure, surface transmittance was defined as the relative arbitrary fraction of intensity that the ray transmits through the surface. In some other embodiments of the disclosure, the relative arbitrary fraction of intensity may be configured to be wavelength dependent. In certain other embodiments of the disclosure, the arbitrary fraction of intensity may be configured to be polarisation sensitive. To evaluate the simulated retinal ganglion cell activity, the spectacle lens was horizontally decentred with respect to the optical axis of the model eye in various decentration positions mimicking saccadic eye movements. The spectacle lens movements with respect to the optical centre of the model eye were contained between ±5 mm in horizontal directions. At each of the decentred spectacle positions, a wide-field retinal image simulation was performed. One-hundred and one (101) such simulated retinal images constituted the input stream for the virtual retinal platform to yield retinal ganglion cell activity. In this example, each of the 101 image frames were configured to be 50 milliseconds accounting for 5.05 seconds of real time stimulation presentation for the virtual retinal model. Each frame of the input stream was configured over 512×512 pixels, wherein each frame was configured to cover the entire diameter of the circular neuron region, encompassing a region approximately, 15°×15° (macular) or 20°×20° (para-macular) of the retina of the virtual retinal platform. The bit depth for each pixel in the input stream was digitised to range from 0 to 255 (i.e. 8-bit). Specific retinal settings and configurations, described in Equations 1 to 9, used to demonstrate the workings for the contact lens embodiments of the present disclosure are discussed in the following section.

In all the Examples 8 to 10, the outer plexiform layer was configured to have a centre region subtending approximately 1.5° (i.e. σC of Equation 2) and a surround region subtending approximately 4.75° (i.e. σS of Equation 3). The centre and surround temporal scales of the outer plexiform layer were set to approximately 1 milli-seconds, represented by variables τC and τS of Equations 2 and 3, respectively. The variables governing the integration centre-surround signals, as described in the Equation 1 herein, were chosen to be w_{OPL}=1 and λ_{OPL}=10. The static nonlinearity coefficients of the bipolar and ganglion cell synapses were fixed across all the Examples 8 to 10. The bipolar linear threshold was set to 0, the linear threshold value was held constant at 80, and the bipolar amplification value held at 100. The values for the neuronal model were maintained across all Examples 8 to 10, wherein a leak of 0.75, neuronal noise of 20, membrane capacitance of 150 and firing threshold of 2.4 were used for the simulations of Examples 8 to 10. The post-synaptic pooling variable Sigma was ignored. The options for a contrast gain control mechanism, utility of the supplementary high-pass filter of the outer plexiform layer and the utility of lateral connectivity of the amacrine cells were kept variable across the Examples 8 to 10. Further details of the specific setting used are disclosed herein.

Advanced optical modelling experiments were conducted using two types of spectacle lenses for each exemplary embodiment described herein: (1) a single vision spectacle lens matched with the base prescription of the schematic model eye to provide correction of the refractive error, which simulates the standard of care; (2) the same standard single vision spectacle lens described above with additional non-refractive features designed in accordance with the invention to provide an increase in the retinal ganglion cell activity compared to the standard of care single vision spectacle lens.

In certain spectacle lens embodiments, the opaque or translucent or absorbing border of the contemplated design features (i.e. aperture) within the optic zone of the spectacle lens may be at least 15 μm, 25 μm, 35 μm, 50 μm, 75 μm, 100 μm, 150 μm, 200 μm or 250 μm in width.

In certain spectacle lens embodiments, the opaque or translucent or absorbing border of the contemplated design features (i.e. aperture) within the optic zone of the spectacle lens may not be configured greater than 300 μm, 325 μm, 350 μm, 375 μm, or 400 μm in width to avoid the potential deterioration of resolution capabilities of the corrected eye and/or to maintain adequate amounts of transmission of light in all viewing conditions for example to accommodate for normal pupil changes between 2 and 7 mm, spanning dim, ambient and high-level of light conditions that may be experienced by the wearer.

Due to the cosmetic appearance of the spectacle lens, a translucent or absorbing/tinted border may be preferred as a design feature over an opaque border. In certain spectacle lens embodiments, the translucent border of the contemplated design features on the spectacle lens may be between 15 and 30 μm, 25 and 50 μm, or 30 and 75 μm, or 15 and 100 μm in width. In some embodiments, the width of the design feature may not be constant across multiple apertures.

In yet another spectacle embodiment the contemplated multiple apertures within the optic zone may be only used when the wearer is doing a particular near visual task, for example, reading a book, writing, playing a video game, using a mobile phone, using a tablet or using a computer.

With respect to the implementation of the contemplated design features in spectacle lenses, in certain embodiments the border of the multiple maybe introduced utilising material that may have polarisation selectivity. Use of such polarisation sensitive material may enhance cosmesis for the wearer yet providing the desirable edge effects to provide a stop signal. Selective test cases may be contemplated (use of liquid crystal display (LCD), light emitting diode display) when using the multiple apertures configure with polarisation sensitive material.

### Example 8—Control (C8) and Exemplary Embodiment (D8) Designs

In this Example 8, the following parameters of the schematic model eye of Table 1 were altered to represent a 3 D myopic eye (i.e. a base prescription Rx of −3 D) in its unaccommodated state: (i) a vitreous chamber depth of the eye of 17.63 mm, and (ii) retinal radius of curvature to 13.5 mm.

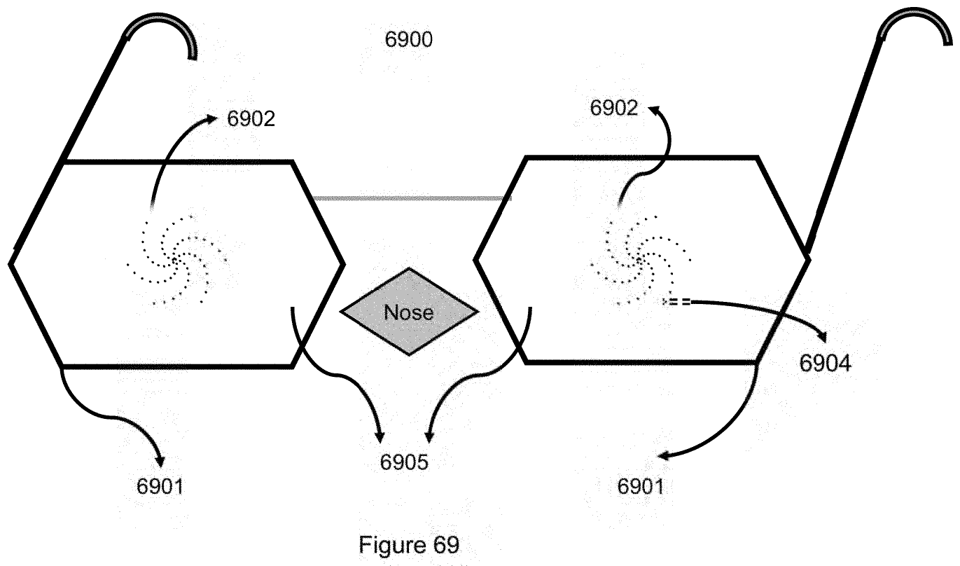

The model was configured to focus on a distant object at optical infinity from the eye. The modified myopic schematic model eye of was corrected, one at time, with the control (C8) and exemplary embodiment (D8) spectacle lenses. The control (C8) spectacle lens represents a single vision lens modelled using the following parameters: front surface (R=2000 mm), a centre thickness (1.5 mm), a back surface (R=144.2 mm) and refractive index of 1.5 with a total blank diameter of 50 mm. The control spectacle lens C8 is free/devoid of any non-refractive features contemplated in this present disclosure. The second lens D8 represents the exemplary embodiment which is also a single vision spectacle lens with same parameters as control C8, that was further configured with non-refractive features disclosed in FIG. 69. The non-refractive features of the exemplary embodiment example D8 (6900) comprises a swirl pattern with 6-arms (6902), each arm further comprises a plurality of dot-like features. The swirl pattern is positioned about the optical centre of the spectacle lens (6901). The total number of dot-like features in each arm (6902) is approximately 10. The total dimensions of the swirl pattern is approximately 5 mm in diameter. The width of the dot-like features is approximately between 75 μm (6904). The remainder of the portion (6905) of the exemplary embodiment D8 is configured with the single vision parameters matching the base prescription of the eye. The non-refractive features of the exemplary embodiment example D8 is configured to such that it absorbs at least 90% of light incident on the non-refractive feature. The simulated retinal images were computed and analysed with control C8 and embodiment D8 spectacle designs when fitted on the schematic model eye of Example 8, following the steps disclosed in paragraphs [00385] to [00387].

In this Example 8, additional variables of the virtual retinal platform were contemplated with the following settings; the option of a contrast gain control mechanism, described in Equations 1, 5 and 6, were used with the following input parameter values: (i) an outer plexiform amplification λ_{OPL} value of 150 Hz per normalised luminance unit; (ii) bipolar inert leaks g_{A}⁰ of 5 Hz; (iii) the feedback amplification λ_{A} of 100 in Hz; (iv) spatial scale σA of 2.5°; and (v) temporal scale τA of 0.01 milli-seconds. The arrangement of the neuronal bundle (1602) was in a circular arrangement spanning 15°×15° field of view.

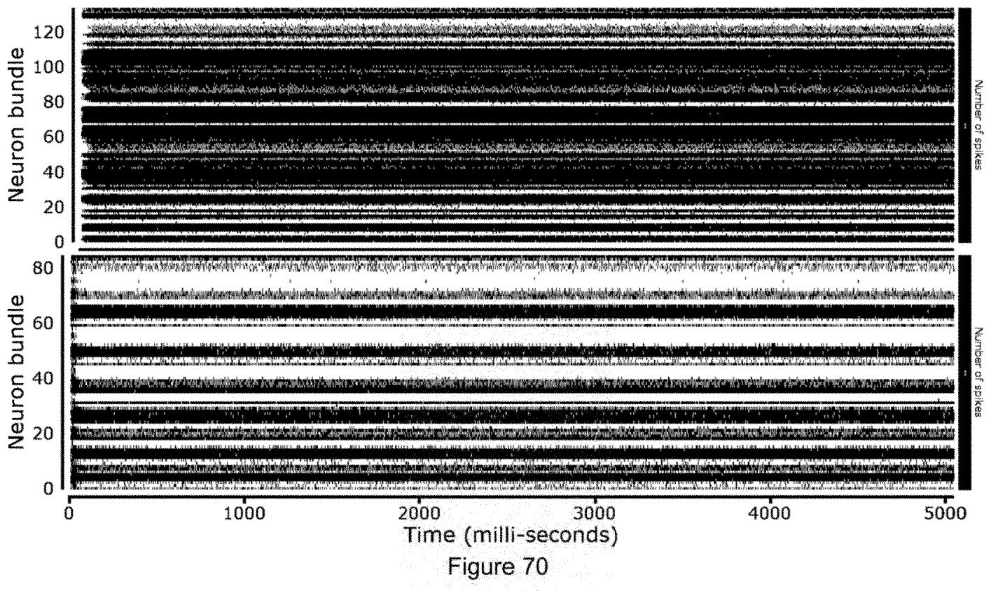

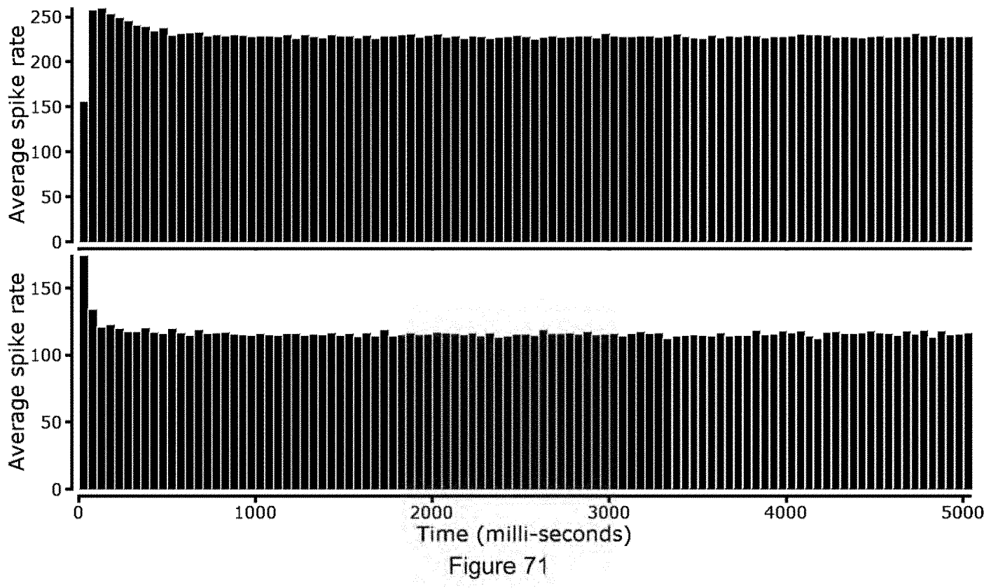

A sparse lateral connectivity mode of the virtual retina was used with 10 pre-synaptic neurons with 10% of positive weight and a weight variance of 0.01. Further, the supplementary high-pass filter option of outer plexiform layer, described in Equations 2 and 3, was used with the following parameters values: a temporal scale of 0.2 milliseconds and spatial scale of 0.5°. The post-synaptic pooling option was also muted. The postprocessing of the computed simulated retinal images of the control (C8) spectacle design of Example 8, using the virtual retinal platform, as discussed herein, results in a spike train as a function of time (FIG. 70) and pen-stimulus histogram highlighting the average spike rate as a function of time (FIG. 71), for cells with both on- and off-polarities. The top and bottom sub-graphs of FIG. 70 and FIG. 71 represent data for on- and off-cells, respectively.

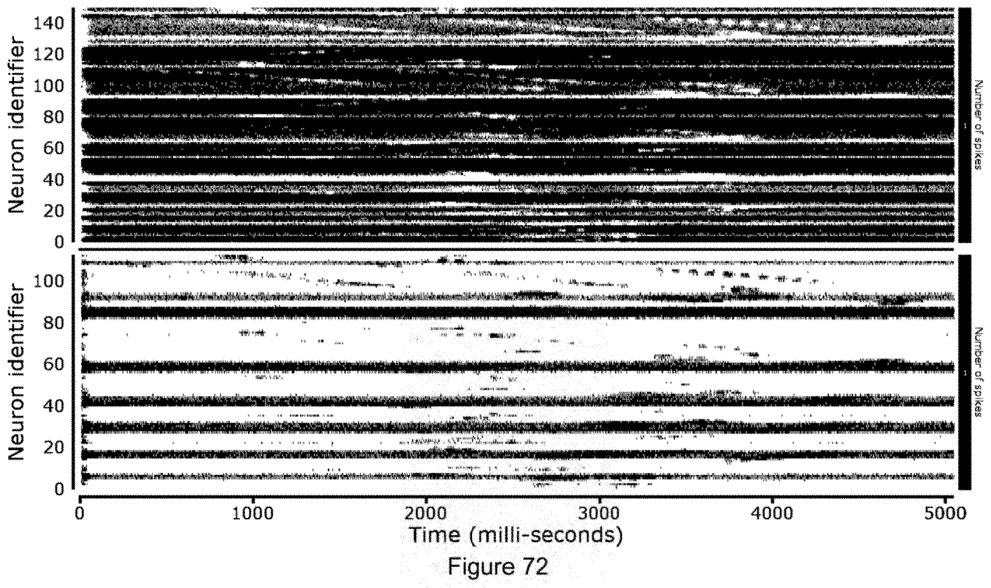

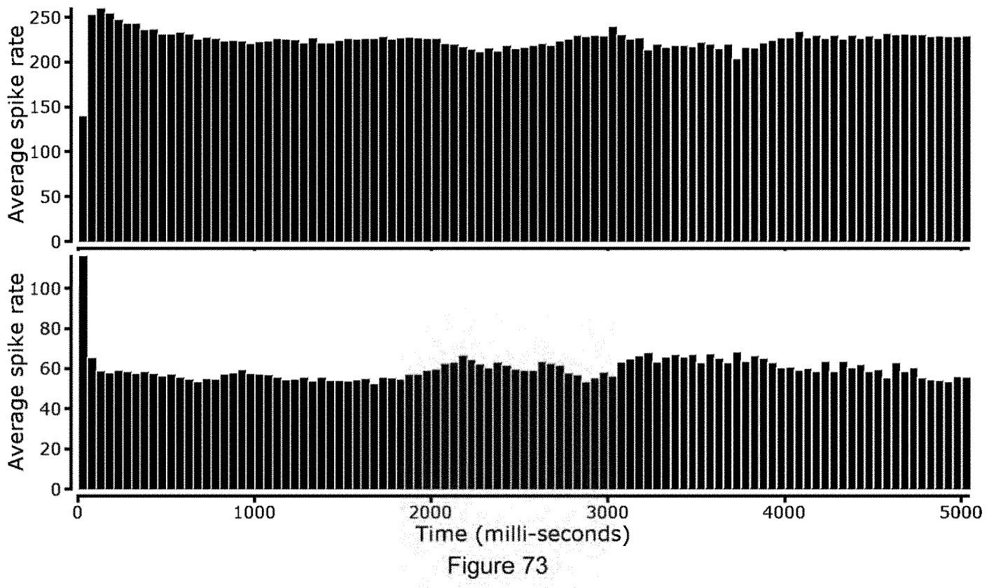

The postprocessing of the computed simulated retinal images of embodiment (D8) spectacle designs of Example 8, using the virtual retinal platform, as discussed herein, results in a spike train as a function of time (FIG. 72) and peri-stimulus histogram highlighting the average spike rate as a function of time (FIG. 73) for cells with both on- and off-polarities. The top and bottom sub-graphs of FIG. 72 and FIG. 73 represent data for on- and off-cells, respectively.

The neuronal activity with the control (C8) spectacle lens, depicted as spike trains of FIG. 70, is relatively time invariant, or with minimal variation, or no variation, or no fluctuation, as a function of time. This observation was similar for cells with both type of polarities, on-type and off-type. On the other hand, the neuronal activity with the embodiment (D8) spectacle lens, depicted as spike trains of FIG. 72, is relatively time variant demonstrating fluctuation with time. The observed fluctuations as a function of time is aperiodic with small amplitudes of the observed fluctuations. The non-stationarity and non-linearity in the spiking responses obtained with the embodiment lens is attributed to the artificial edges, or luminous contrast profiles, in the retinal imagery, or temporal variation of the artificial edges.

In the Example 8, the neuronal activity with the control (C8) spectacle lens, depicted as average spike rate of FIG. 71, follows a relatively monotonous profile following the initial 100 milliseconds, signifying stabilisation of the signal. This observed pattern is similar for cells with both type of polarities, on- and off.

On the other hand, the neuronal activity with embodiment (D8) spectacle lens, depicted as average spike rate of FIG. 73, follows a time-variant pattern for both on-type and off-type cells. In this Example 8, the on- and off-axis evaluation of the optical performance, was modelled in polychromatic mode, spanning 470 nm to 650 nm wavelengths, using a luminosity function to describe average spectral sensitivity of human visual perception of brightness in photopic condition at 6 mm pupil analysis diameter.

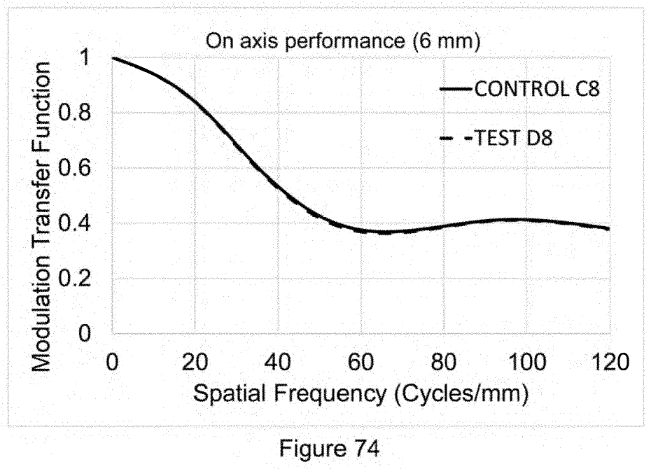

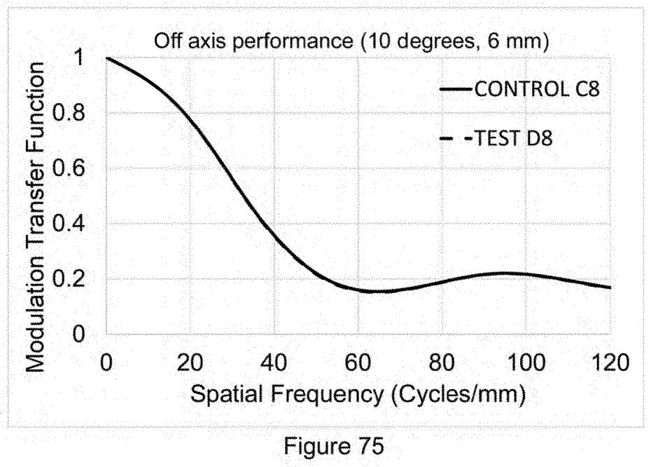

As described herein FIGS. 74 and 75, the wide-field optical performance, as gauged using modulation transfer function as function of spatial frequencies at 6 mm pupil diameter, between the control (C8) and exemplary embodiment (D8) spectacle lenses is virtually indistinguishable, represented by the solid black line and dashed black lines. For the off-axis performance, in Example 8, the field of view considered for evaluation of performance was 20°, which is ±10° from the centre.

### Example 9—Control (C9) and Exemplary Embodiment (D9) Designs

In this Example 9, the following parameters of the schematic model eye of Table 1 were altered to represent a 1 D myopic eye (i.e. a base prescription Rx of −3 D) in its 1D accommodated state: (i) a vitreous chamber depth of the eye of 16.92 mm, (ii) retinal radius of curvature to 12 mm; and (iii) anterior lens radius (R=9.34 mm) and conic constant (Q=−3.2) parameters.

The model was configured to focus on a distant object at 1 metre away the eye. The modified myopic schematic model eye of was corrected, one at time, with the control (C9) and exemplary embodiment (D9) spectacle lenses. The control (C8) spectacle lens represents a single vision lens modelled using the following parameters: front surface (R=2000 mm), a centre thickness (1.5 mm), a back surface (R=379.1 mm) and refractive index of 1.5 with a total blank diameter of 50 mm. The control spectacle lens C9 is free/devoid of any non-refractive features contemplated in this present disclosure.

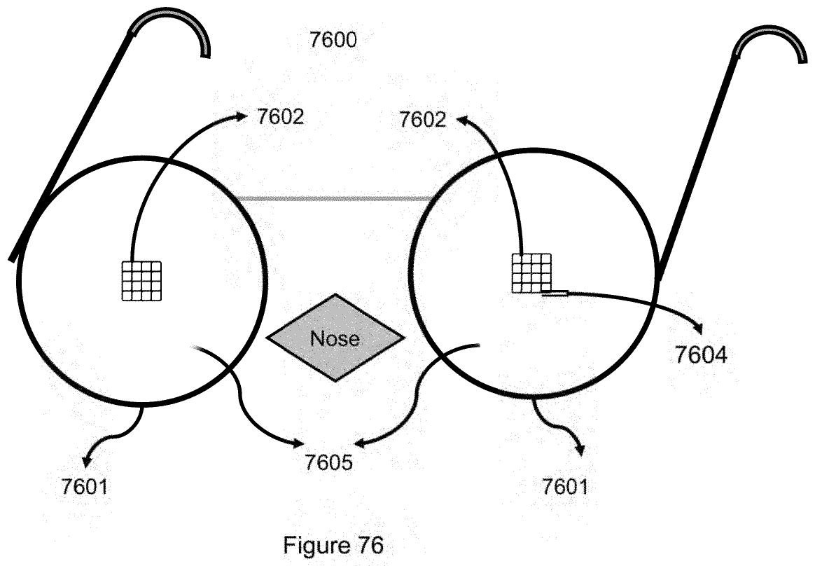

The second lens D9 represents the exemplary embodiment which is also a single vision spectacle lens with same parameters as control C9, that was further configured with non-refractive features disclosed in FIG. 76.

The non-refractive features of the exemplary embodiment example D9 comprises a square-grid pattern (7602) which further comprises a plurality of square apertures, positioned about the optical centre of the spectacle lens (7601). The total number of apertures designed within the pattern (7602) is approximately 16. The total dimensions of the square-grid is approximately 3×3 mm. The width of the lines, or border forming of the square aperture is approximately between 50 μm (7604). The remainder of the portion (7605) of the exemplary embodiment D9 is configured with the single vision parameters matching the base prescription of the eye. The non-refractive features of the exemplary embodiment example D9 is configured to such that it absorbs at least 85% of light incident on the non-refractive feature.

The simulated retinal images were computed and analysed with the control C9 and embodiment D9 spectacle designs when fitted on the schematic model eye of Example 9, following the steps disclosed in paragraphs [00385] to [00387]. In this Example 9, additional variables of the virtual retinal platform were contemplated with the following settings; the option of a contrast gain control mechanism, described in Equations 1, 5 and 6, was. The arrangement of the neuronal bundle (1602) was in a circular arrangement spanning 20°×20° field of view.

A sparse lateral connectivity mode of the virtual retina was used with 10 pre-synaptic neurons with 10% of positive weight and a weight variance of 0.01. The supplementary high-pass filter option of outer plexiform layer, described in Equations 2 and 3, was muted. The post-synaptic pooling option was also muted

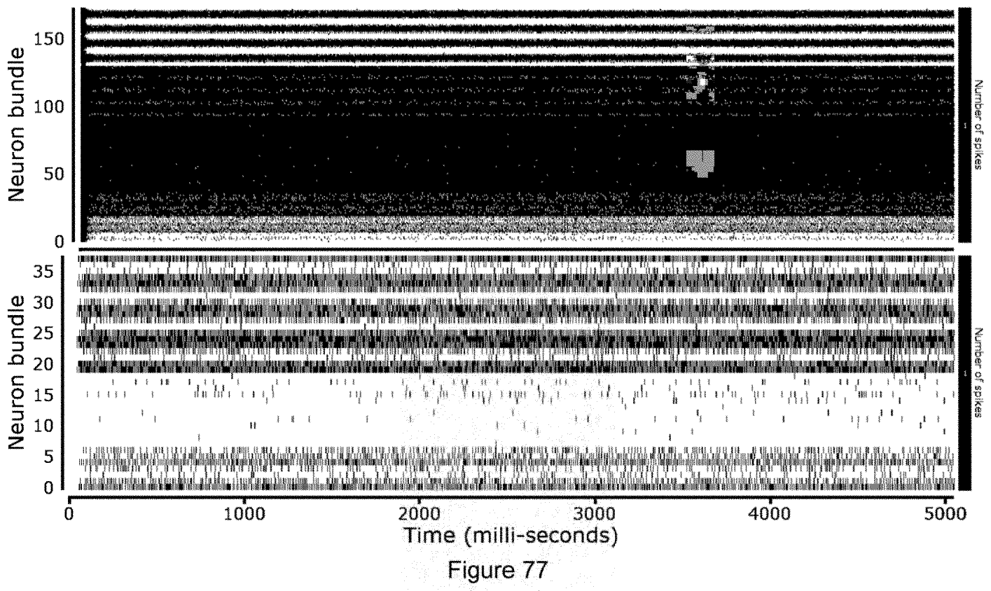

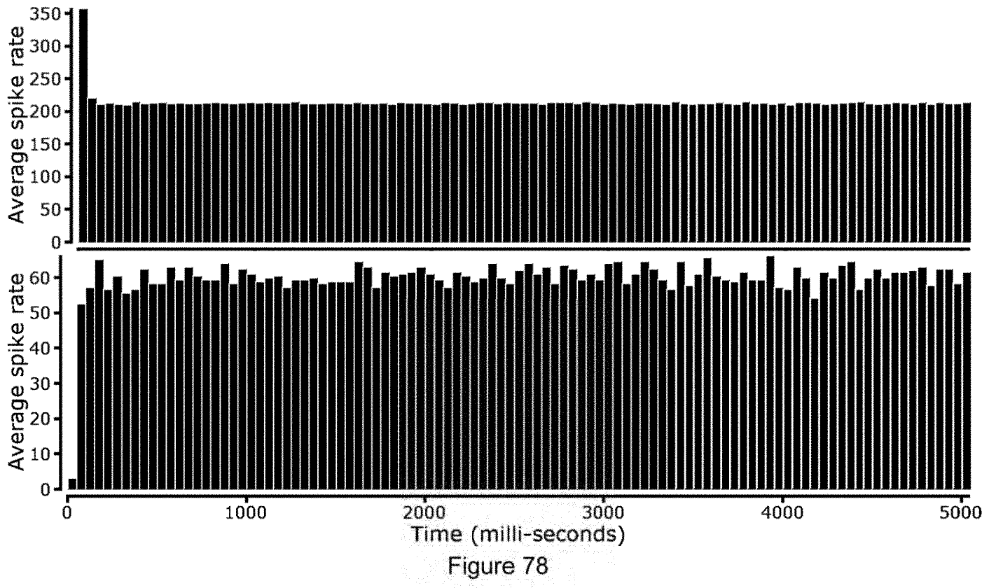

The postprocessing of the computed simulated retinal images of the control (C9) spectacle design of Example 9, using the virtual retinal platform, as discussed herein, results in a spike train as a function of time (FIG. 77) and peri-stimulus histogram highlighting the average spike rate as a function of time (FIG. 78), for cells with both on-type and off-type polarities. The top and bottom sub-graphs of FIG. 77 and FIG. 78 represent data for on- and off-cells, respectively.

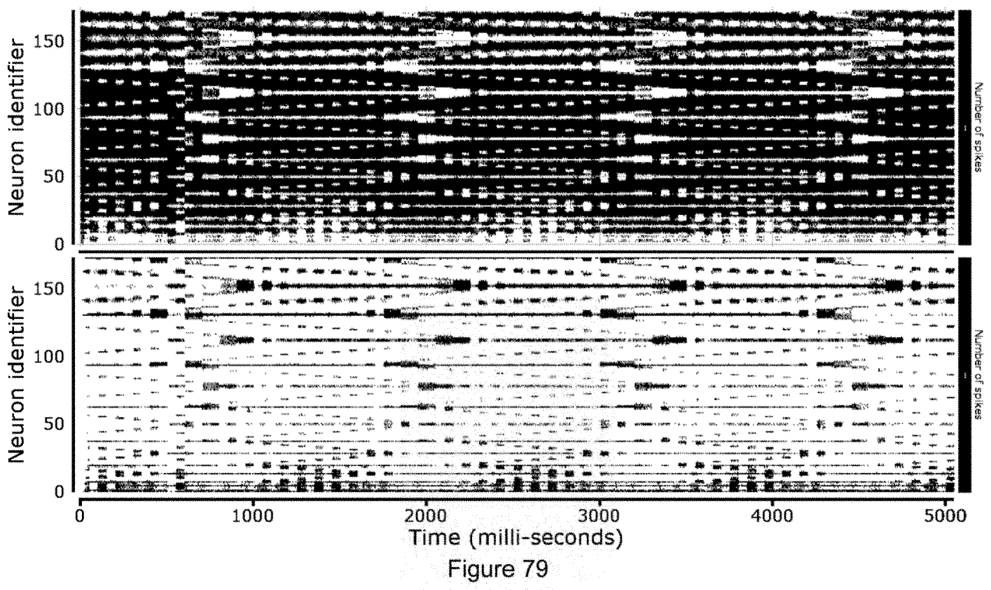

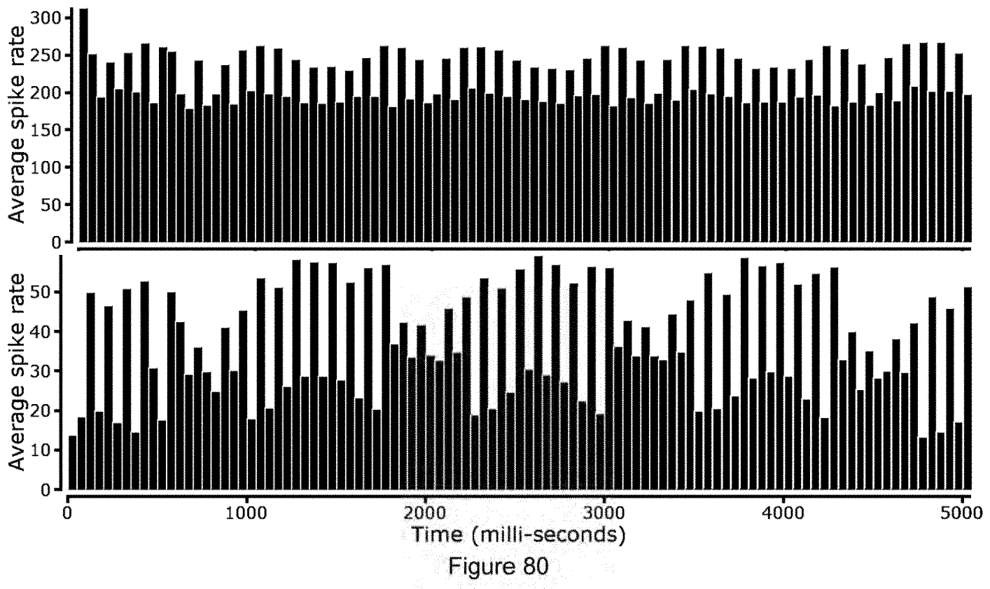

The postprocessing of the computed simulated retinal images of the embodiment (D9) spectacle designs of Example 9, using the virtual retinal platform, as discussed herein, results in a spike train as a function of time (FIG. 79) and peri-stimulus histogram highlighting the average spike rate as a function of time (FIG. 80) for cells with both on-type and off-type polarities. The top and bottom sub-graphs of FIG. 79 and FIG. 80 represent data for on-type and off-type cells, respectively.

The neuronal activity with the control (C9) spectacle lens, depicted as spike trains of FIG. 77, is relatively time invariant or with minimal variation or fluctuation as a function of time, for cells with both type of polarities, on- and off. On the other hand, the neuronal activity with the embodiment (D9) spectacle lens, depicted as spike trains of FIG. 79, is relatively time variant and fluctuating with varying periodicity as a function of time. In the Example 9, the neuronal activity with the control (C9) spectacle lens, depicted as average spike rate of FIG. 78, follows a relatively monotonous profile following the initial 50 milliseconds, signifying stabilisation of the signal. This observed pattern is similar for cells with both type of polarities, on-type, and off-type. The off-type cell response did show some variability of the average spike rate as a function of time; however, the magnitude of the change were small in magnitude. On the other hand, the neuronal activity with the embodiment (D9) spectacle lens, depicted as the average spike rate as function of time, described in FIG. 80, obtained with the embodiment (D9) spectacle lens follows a time-variant pattern for both on-type and off-type cells. The neuronal activity with the control (C9) spectacle lens, depicted as spike trains of FIG. 77, is relatively time invariant, for both type of polarities. The non-stationarity and non-linearity in the spiking responses obtained with the embodiment lens is attributed to the artificial edges, or luminous contrast profiles, in the retinal imagery, or temporal variation of the artificial edges.

As can be seen from the responses of discrete neuronal bundles, the number of active discrete off-type neuron bundles are 3 to 4 times lower in number than the corresponding active discrete on-type neuron bundles. On the other hand, the neuronal activity with the embodiment (D9) spectacle lens, depicted as spike trains of FIG. 79, is relatively time variant for both type of polarities. Further, the total number of active off-type discrete neuron bundles were comparable to the number of active on-type discrete neuron bundles.

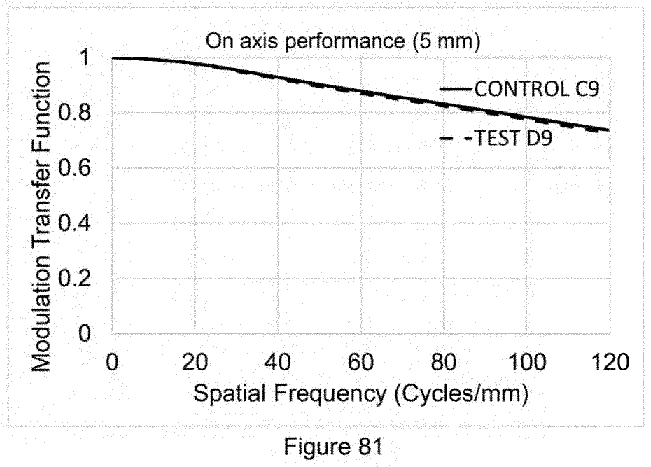

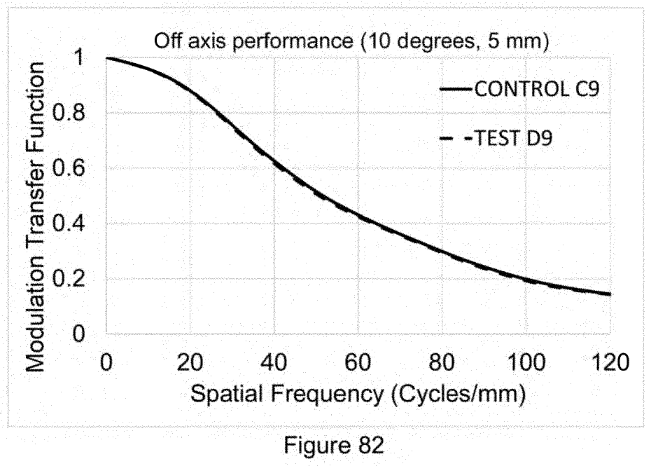

In this Example 9, the on- and off-axis evaluation of the optical performance, was modelled in monochromatic mode (589 nm) and at 5 mm pupil analysis diameter. As described herein FIGS. 81 and 82, the wide-field optical performance, as gauged using modulation transfer function as function of spatial frequencies at 5 mm pupil diameter, between the control (C9) and exemplary embodiment (D9) spectacle lenses is virtually indistinguishable. For the off-axis performance, in Example 9, the field of view considered for evaluation of performance was 20°, which is ±10° from the centre.

### Example 10—Control (C10) and Exemplary Embodiment (D10) Designs

In this Example 10, the following parameters of the schematic model eye of Table 1 were altered to represent a 4 D myopic eye (i.e. a base prescription Rx of −4 D) in its 2D accommodated state: (i) a vitreous chamber depth of the eye of 18 mm, (ii) retinal radius of curvature to 12 mm; and (iii) anterior lens radius (R=7.934 mm) and conic constant (Q=−1.962) parameters.

The model was configured to focus on a distant object at 50 cm away the eye. The modified myopic schematic model eye of was corrected, one at time, with the control (C10) and exemplary embodiment (D10) spectacle lenses. The control (C10) spectacle lens represents a single vision lens modelled using the following parameters: front surface (R=2000 mm), a centre thickness (1.5 mm), a back surface (R=102.26 mm) and refractive index of 1.5 with a total blank diameter of 50 mm. The control spectacle lens C10 is free/devoid of any non-refractive features contemplated in this present disclosure.

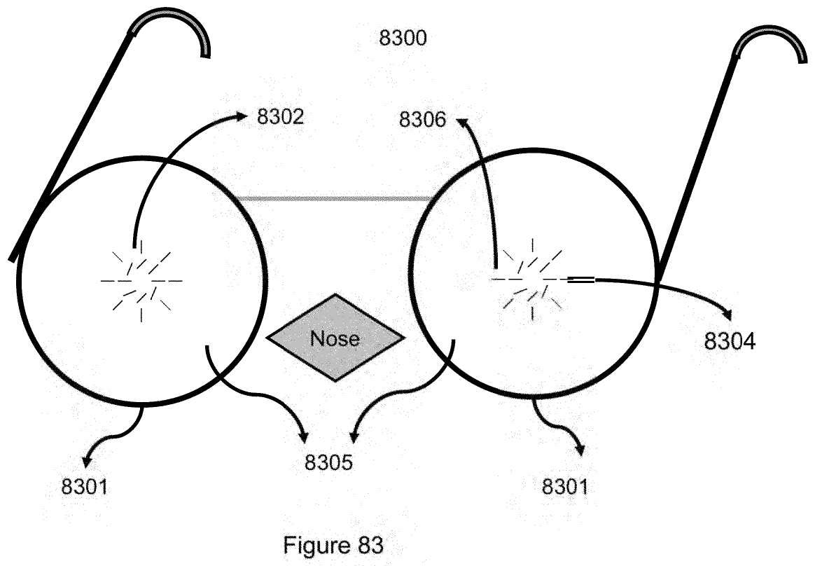

The second lens D10 represents the exemplary embodiment which is also a single vision spectacle lens with same parameters as control C10, that was further configured with non-refractive features disclosed in FIG. 83. The non-refractive features of the exemplary embodiment example D10 comprises a non-refractive feature configured as a random pattern (8302) which further comprises a series of lines, or striae, positioned about the optical centre of the spectacle lens (8301). The total number of lines, or striae, designed within the pattern (8302) is approximately 16. The length of the lines, or striae (8306) is approximately between 0.75 mm and 1.25 mm.

The width of the lines, or striae (8304) is approximately between 25 μm and 75 μm. The remainder of the portion (8305) of the exemplary embodiment D10 is configured with the single vision parameters matching the base prescription of the eye. The non-refractive features of the exemplary embodiment example D10 is configured to such that it absorbs at least 80% of light incident on the non-refractive feature.

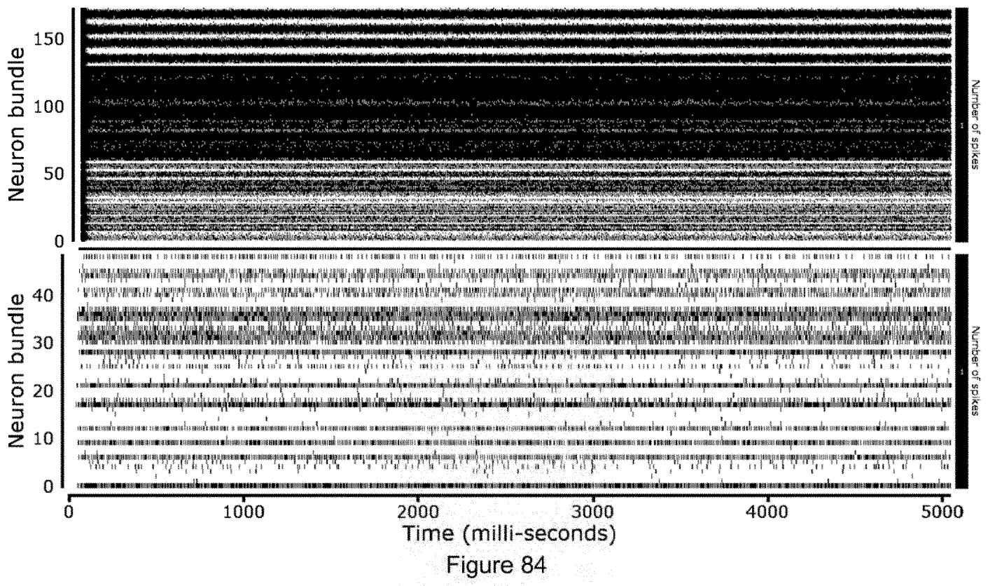

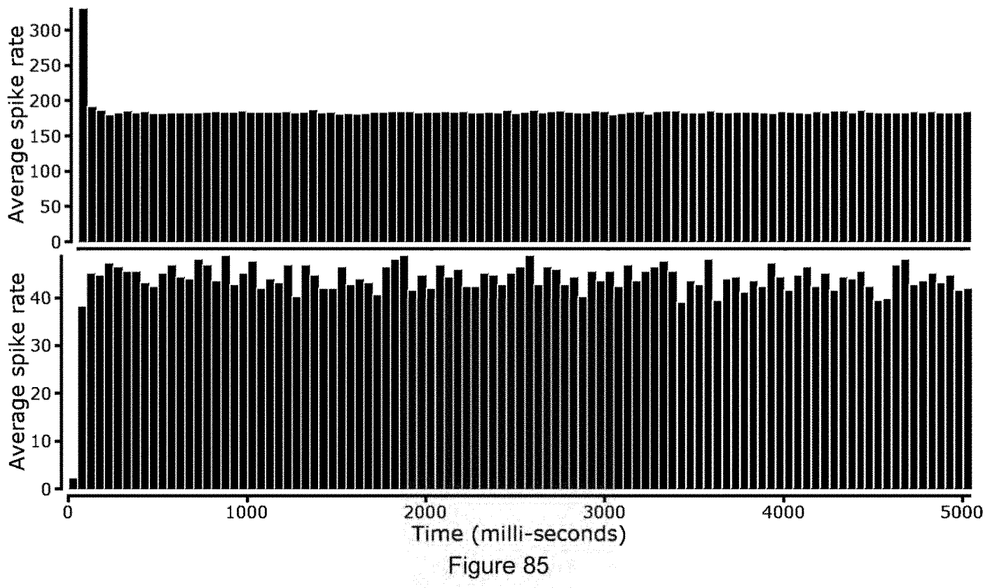

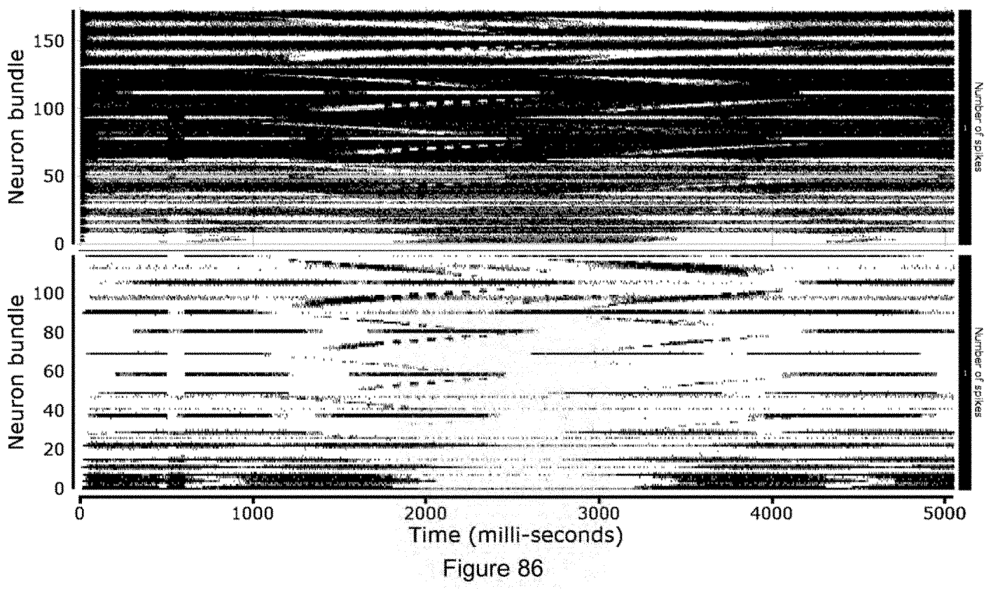

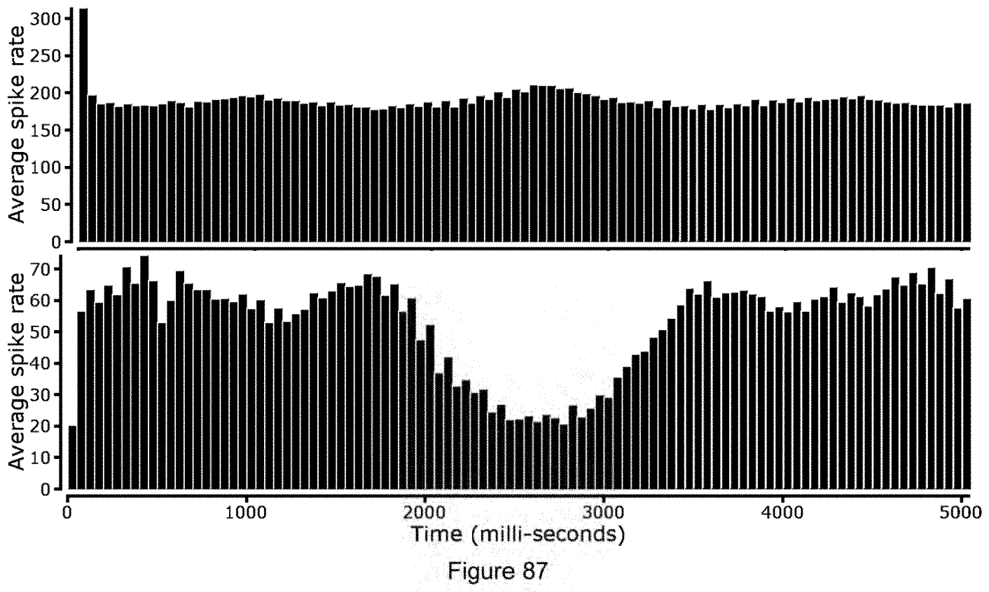

The simulated retinal images were computed and analysed with the control C10 and the embodiment D10 spectacle designs, when fitted, one at a time, on the schematic model eye of Example 10, following the steps disclosed in paragraphs [00385] to [00387]. In this Example 10, additional variables of the virtual retinal platform were contemplated with the following settings; the option of a contrast gain control mechanism, described in Equations 1, 5 and 6, was. The arrangement of the neuronal bundle (1602) was in a circular arrangement spanning 20°×20° field of view. A sparse lateral connectivity mode of the virtual retina was muted. The supplementary high-pass filter option of outer plexiform layer, described in Equations 2 and 3, was muted. The post-synaptic pooling option was also muted. The postprocessing of the computed simulated retinal images of the control (C10) spectacle design of Example 10, using the virtual retinal platform, as discussed herein, results in a spike train as a function of time (FIG. 84) and pen-stimulus histogram highlighting the average spike rate as a function of time (FIG. 85), for cells with both on-type and off-type polarities. The top and bottom sub-graphs of FIG. 84 and FIG. 85 represent data for on-type and off-type cells, respectively. The postprocessing of the computed simulated retinal images of the embodiment (D10) spectacle designs of Example 10, using the virtual retinal platform, as discussed herein, results in a spike train as a function of time (FIG. 86) and pen-stimulus histogram highlighting the average spike rate as a function of time (FIG. 87) for cells with both on- and off-type polarities.

The top and bottom sub-graphs of FIG. 86 and FIG. 87 represent data for on-type and off-type cells, respectively. The neuronal activity with the control (C10) spectacle lens, depicted as spike trains of FIG. 84, is relatively time invariant, for both type of polarities, i.e. on-type (top sub-graph of FIG. 84) and off-type cells (bottom sub-graph of FIG. 84). The Y-axis of the sub-graphs represent the responses of discrete neuronal bundles.

As can be seen, the number of active discrete off-type neuron bundles are 3 to 4 times lower in number than the corresponding active discrete on-type neuron bundles. On the other hand, the neuronal activity with the embodiment (D10) spectacle lens, depicted as spike trains of FIG. 86, is relatively time variant for both type of polarities, i.e. on-type (top sub-graph of FIG. 86) and off-type cells (bottom sub-graph of FIG. 86). With the embodiment (D10) spectacles lens example however, the total number of active off-type discrete neuron bundles were comparable to the number of active on-type discrete neuron bundles.

In the Example 10, the neuronal activity with the control (C10) spectacle lens, depicted as average spike rate of FIG. 85, follows a relatively monotonous profile following the initial 50 milliseconds, signifying stabilisation of the signal for the on-type cells (top-graph of FIG. 85). On the hand, the off-type cells, demonstrated a small variation in the average spike rate as a function of time, however these variations were small in magnitude.

Contrastingly different, the discrete neuronal activity with the embodiment (D10) spectacle lens, depicted as average spike rate in FIG. 87, is variable as a function of time. The time-variant pattern is observed in both on-type and off-type cells; however, it is greater in magnitude with the off-type cells. The pattern observed in the off-type cells (bottom graph of FIG. 87) between the time points 2000 and 3000 milli-seconds, the average spike rate follows a quasi-sinusoidal pattern. At various other time points of the off-type cell response, the quasi-sinusoidal pattern diminishes in its amplitude. The on-type cell response also demonstrates the variations in the average spike rate as a function of time however the magnitude of the variation is lower.

The non-stationarity and non-linearity in the spiking responses obtained with the embodiment lens is attributed to the artificial edges, or luminous contrast profiles, in the retinal imagery, or temporal variation of the artificial edges. In the Example 10, the on- and off-axis evaluation of the optical performance, was modelled in polychromatic mode, spanning 470 nm and 650 nm wavelengths, using a luminosity function describing average spectral sensitivity of human visual perception of brightness in photopic condition at 4 mm pupil analysis diameter.

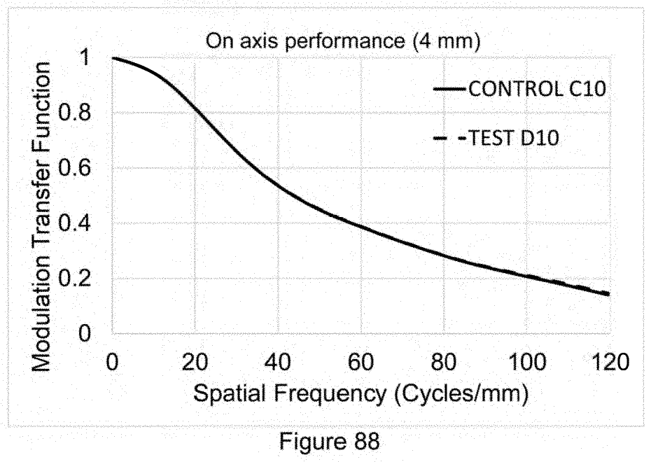

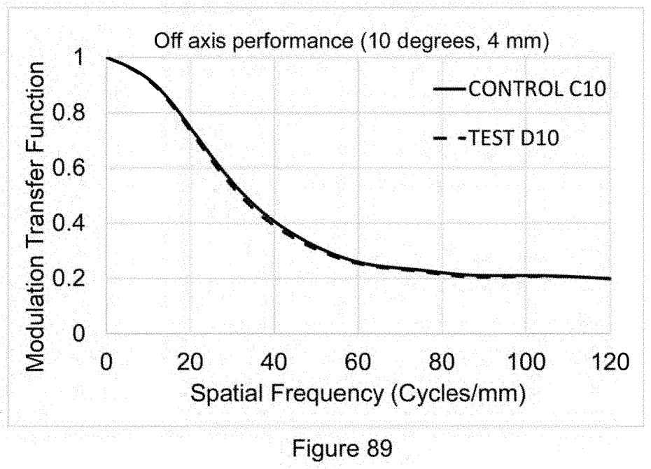

As described herein FIGS. 88 and 89, the wide-field optical performance, as gauged using modulation transfer function as function of spatial frequencies at 4 mm pupil diameter, between the control (C10) and exemplary embodiment (D10) spectacle lenses is substantially similar, represented by the solid black line and dashed black lines. For the off-axis performance, in Example 10, the field of view considered for evaluation of performance was 20°, which is ±10° from the centre.

### Example Claim Set A

A contact lens for an eye, the contact lens comprising: a front surface; a back surface; an optic zone including: a base prescription providing substantial correction for the distance refractive error of the eye, and a plurality of non-refractive features; and a peripheral zone surrounding the optic zone.

The contact lens of the above claim example of set A, wherein the base prescription for the eye includes at least one of the following: a spherical correction, an astigmatic correction, or a spherical and an astigmatic correction.

The contact lens of one or more of the above claim examples of set A, wherein the plurality of non-refractive features comprises at least one of the following: a plurality of substantially opaque borders forming a plurality of apertures, wherein each aperture circumscribes a substantially transparent region, or a plurality of substantially opaque features forming one or more patterns without a substantially distinct border.

The contact lens of one or more of the above claim examples of set A, wherein each substantially transparent region comprises the base prescription for the eye.

The contact lens of one or more of the above claim examples of set A, wherein the shape of at least one of the plurality of apertures is circular, elliptical, oval, triangular, rectangular, square, pentagonal, or hexagonal, or octagonal, or any other regular polygon, or an irregular polygon, or a random shape.

The contact lens of one or more of the above claim examples of set A, wherein the plurality of apertures are configured in a circular, hexagonal, radial, spiral, regular, irregular, or random arrangement.

The contact lens of one or more of the above claim examples of set A, wherein the surface area of the circumscribed transparent region of at least one of the plurality of apertures is between 0.25 sq mm and 2.5 sq mm, or between 0.5 sq mm and 5 sq mm, or between 0.75 sq mm and 7.5 sq mm, or between 0.25 sq mm and 7.5 sq mm.

The contact lens of one or more of the above claim examples of set A, wherein the widths of the substantially opaque border of any of the plurality of apertures is at least 3, at least 4, or at least 6, or at least 8, or at least 10 times the mean wavelength of the visible spectrum of light (i.e. 555 nm) such that the substantially opaque border remain substantially non-diffractive.

The contact lens of one or more of the above claim examples of set A, wherein the widths of the substantially opaque border of any of the plurality of apertures is between 5 μm to 75 μm, or between 25 μm to 150 μm, or between 50 μm to 250 μm.

The contact lens of one or more of the above claim examples of set A, wherein the total number of apertures in the plurality of apertures is at least 2, at least 3, at least 4, at least 5, at least 6, or at least 7 apertures.

The contact lens of one or more of the above claim examples of set A, wherein the plurality of patterns without a substantially distinct border includes at least: a spoke wheel pattern, a spiral pattern, a swirl pattern, a grid pattern, a Memphis pattern, a dot-like pattern, a regular pattern, an irregular pattern, a moiré fringe pattern, an interference pattern, a random pattern with dots, a random pattern with straight lines, a random pattern with non-circular dots, a random pattern with curvilinear lines, a random pattern with arcs, a random pattern with zig-zag lines; wherein each pattern of the plurality of patterns is formed with substantially opaque features comprising of dots, lines or striae.

The contact lens of one or more of the above claim examples of set A, wherein plurality of patterns without a substantially distinct border is centred, or decentred, within the optic zone.

The contact lens of one or more of the above claim examples of set A, wherein the total surface area of the plurality of non-refractive features occupies between 2.5 percent and 10 percent, or between 5 percent and 15 percent, or 7.5 percent and 20 percent of the total surface area of the optic zone.

The contact lens of one or more of the above claim examples of set A, wherein the plurality of non-refractive features are configured to be within the central 3 mm, or central 4 mm, or central 5 mm, or central 6 mm, of the optic zone.

The contact lens of one or more of the above claim examples of set A, wherein region outside the central 6.5 mm, or outside the central 7 mm or outside the central 7.5 mm, of the optic zone is substantially devoid of the non-refractive features.

The contact lens of one or more of the above claim examples of set A, wherein the plurality of non-refractive features is applied on the front surface, or the back surface, or both front and back surfaces.

The contact lens of one or more of the above claim examples of set A, wherein the plurality of non-refractive features is applied within the matrix of the contact lens.

The contact lens of one or more of the above claim examples of set A, wherein the total light transmittance through the optic zone is between 85 percent and 90 percent, or between 90 percent and 95 percent, or between 92.5 percent and 97.5 percent, or between 85 percent and 99 percent of the total light transmittance through the optic zone of a similar single vision lens devoid of the non-refractive features.

The contact lens of one or more of the above claim examples of set A, wherein the plurality of non-refractive features is configured at least in part to be sensitive to polarisation of the incident light.

The contact lens of one or more of the above claim examples of set A, wherein the plurality of non-refractive features are activated and turn opaque, at least in part, when the incident light is linearly, or circularly, or elliptically polarised.

The contact lens of one or more of the above claim examples of set A, wherein the plurality of non-refractive features are activated and turn opaque, at least in part, when the incident light is coming from a LCD or a LED or an OLED monitor screen, TV screen, tablet screen, or mobile screen or a screen of similar electronic devices.

The contact lens of one or more of the above claim examples of set A, wherein the plurality of non-refractive features is configured at least in part to be electronically tuneable.

The contact lens of one or more of the above claim examples of set A, wherein the non-refractive features are configured such that the material characteristics are spectrally sensitive to certain visible wavelengths between 420 to 760 nm, inclusive.

The contact lens of one or more of the above claim examples of set A, wherein the lens is capable of providing the wearer with adequate visual performance that is substantially similar to that obtained with a single vision lens devoid of non-refractive features.

The contact lens of one or more of the above claim examples of set A, wherein the non-refractive features are configured such that the material characteristics are spectrally sensitive to certain visible wavelengths between 420 to 760 nm

The contact lens of one or more of the above claim examples of set A, when tested on a model eye configured with a distance refractive error substantially matching the base prescription, provides an on-axis modulation transfer function, for at least one pupil between 3 mm and 6 mm inclusive, and at least one wavelength 420 nm to 760 nm inclusive, which is substantially equivalent to that obtained with a single vison contact lens that is devoid of the non-refractive features.

The contact lens of one or more of the above claim examples of set A, when tested on a model eye configured with a distance refractive error substantially matching the base prescription, provides an off-axis wide-field modulation transfer function, for at least one pupil between 3 mm and 6 mm inclusive, and at least one wavelength 420 nm to 760 nm inclusive, which is substantially equivalent to that obtained with a single vison contact lens that is devoid of the non-refractive features.

The contact lens of one or more of the above claim examples of set A, wherein the wide-field of the retina includes at least 5°, or 10°, or 15°, or 20°, or 25°, or 30°, of the visual field.

The contact lens of one or more of the above claim examples of set A, when tested on a model eye configured with a distance refractive error substantially matching the base prescription, provides substantial correction of the distance refractive error of the eye and results in artificial edges, or spatial luminous contrast profiles, spread across the wide-field of the retina of the model eye.

The contact lens of one or more of the above claim examples of set A, when tested on a model eye configured with a distance refractive error substantially matching the base prescription, in various decentration positions to emulate one of the following: on-eye movement of the contact lens; eye movements of the wearer, or combination thereof, provides a temporal variation of the artificial edges, or spatial luminous contrast profiles, spread across the wide-field of the retina of the model eye.

The contact lens of one or more of the above claim examples of set A, wherein the model eye is a schematic, a physical, or a bench-top model eye.

The contact lens of one or more of the above claim examples of set A, when tested on a bench-top, or a physical, model eye configured with a distance refractive error substantially matching the base prescription, results in a substantial correction of the distance refractive error of the model eye.

The contact lens of one or more of the above claim examples of set A, wherein the retina of the bench-top, or physical, model eye comprising a camera with, a charge coupled device, or a complementary metal oxide sensor, is configured to capture images of a visual scene projected through the model eye corrected with the contact lens.

The contact lens of one or more of the above claim examples of set A, wherein the images captured by the retina of the model eye serves as an input stream for the virtual retinal simulator comprising at least one of the three image processing steps disclosed herein: (a) spatiotemporal filtering of the input stream of images resulting in a band-pass current, (b) an instantaneous non-linear contrast gain control using variable feedback gate shunt conductance, and (c) a discrete set of noisy integrate-and-fire cell models, resulting in spike trains depicting ganglion cell activity.

The contact lens of one or more of the above claim examples of set A, wherein the plurality of non-refractive regions are configured to provide an increase in the retinal ganglion cell activity compared to that obtained with a single vison contact lens that is devoid of the non-refractive features.

The contact lens of one or more of the above claim examples of set A, wherein the retinal ganglion cell activity, gauged as mean retinal spike rate integrated over a certain time frame, is at least 1.25 times, 1.5 times, 1.75 times, 2 times, 2.25 times, 2.5 times, 2.75 times, 3 times of the retinal ganglion cell activity of a single vison contact lens that is devoid of the non-refractive features.

The contact lens of one or more of the above claim examples of set A, the certain time frame over which the mean retinal spike rate is integrated may be at least 1 second, or at least 3 seconds, or at least 10 seconds, or at least 30 seconds, or at least 60 seconds, or at least 120 seconds, or at least 180 seconds.

The contact lens of one or more of the above claim examples of set A, wherein the retinal ganglion cell activity, or non-stationarity in neural response, gauged as mean retinal spike rate, is observed in on-centre/off-surround retinal field, or on-surround/off-centre retinal field, or both.

The contact lens of one or more of the above claim examples of set A, wherein the function describing the overall retinal ganglion cell activity at the retina of the model eye, or non-stationarity in neural response, gauged in terms of mean retinal spike rate as a function of time, follows a non-linear, or aperiodic, or a sinusoidal, or quasi-sinusoidal, rectangular-wave, quasi rectangular-wave, square-wave, quasi square-wave, or a non-monotonic, pattern depicting a temporal variation in the overall retinal ganglion cell activity.

The contact lens of one or more of the above claim examples of set A, wherein the plurality of non-refractive regions provide at least one of slowing, retarding, or preventing myopia progression, measured by change in axial length or distance refractive error of the eye.

The contact lens of one or more of the above claim examples of set A, where in the contact lens provides, at least in part, adequate foveal correction for the refractive error of the eye, and the non-refractive features provide, at least in part, a time-varying and/or spatially variant stop signal to reduce the rate of myopia progression.

The contact lens of one or more of the above claim examples of set A, wherein the effect of at least one of slowing, retarding, or preventing myopia progression is maintained across at least 12, 24, 36, 48, or 60 months of lens wear.

The contact lens of one or more of the above claim examples of set A, wherein peripheral region is devoid of the plurality of substantially opaque features.

The contact lens of one or more of the above claim examples of set A, wherein the non-refractive features are applied using pad-printing, laser etching, photo-etching, or laser printing.

The contact lens of one or more of the above claim examples of set A combined with one or more of the spectacle lens claim examples of Set B constitute additional embodiments.

### Example Claim Set B

A spectacle lens for an eye, the spectacle lens comprising: a front convex surface; a back concave surface; an optical centre about which a base prescription is configured to provide a substantial correction for the distance refractive error of the eye, and further comprises a plurality of non-refractive features.

The spectacle lens of the above claim example of set B, wherein the base prescription for the eye includes at least one of the following: a spherical correction, an astigmatic correction, or a spherical and an astigmatic correction.

The spectacle lens of one or more of the above claim examples of set B, wherein the plurality of non-refractive features comprises at least one of the following: a plurality of substantially opaque borders forming a plurality of apertures, wherein each aperture circumscribes a substantially transparent region, or a plurality of substantially opaque features forming one or more patterns without a substantially distinct border.

The spectacle lens of one or more of the above claim examples of set B, wherein each substantially transparent region comprises the base prescription for the eye.

The spectacle lens of one or more of the above claim examples of set B, wherein the shape of at least one of the plurality of apertures is circular, elliptical, oval, triangular, rectangular, square, pentagonal, or hexagonal, or octagonal, or any other regular polygon, or an irregular polygon, or a random shape.

The spectacle lens of one or more of the above claim examples of set B, wherein the surface area of the circumscribed transparent region of at least one of the plurality of apertures is between 0.25 sq mm and 2.5 sq mm, or between 0.5 sq mm and 5 sq mm, or between 0.75 sq mm and 7.5 sq mm, or between 0.25 sq mm and 7.5 sq mm.

The spectacle lens of one or more of the above claim examples of set B, wherein the widths of the substantially opaque border of any of the plurality of apertures is at least 3, or at least 4, or at least 6, or at least 8, or at least 10 times the mean wavelength of the visible spectrum of light (i.e. 555 nm) such that the substantially opaque border remain substantially non-diffractive.

The spectacle lens of one or more of the above claim examples of set B, wherein the widths of the substantially opaque border of any of the plurality of apertures is between 5 μm to 75 μm, or between 25 μm to 150 μm, or between 50 μm to 250 μm.

The spectacle lens of one or more of the above claim examples of set B, wherein the total number of apertures in the plurality of apertures is at least 6, at least 9, at least 12, at least 18, at least 24, or at least 30 apertures.

The spectacle lens of one or more of the above claim examples of set B, wherein the plurality of apertures are configured in a circular, hexagonal, radial, spiral, regular, irregular, or random arrangement.

The spectacle lens of one or more of the above claim examples of set B, wherein the plurality of patterns without a substantially distinct border includes at least: a spoke wheel pattern, a spiral pattern, a swirl pattern, a grid pattern, a Memphis pattern, a dot-like pattern, a regular pattern, an irregular pattern, a moiré fringe pattern, an interference pattern, a random pattern with dots, a random pattern with straight lines, a random pattern with curvilinear lines, a random pattern with arcs, a random pattern with zig-zag lines, wherein each pattern of the plurality of patterns is formed with substantially opaque features comprising of dots, lines or striae.

The spectacle lens of one or more of the above claim examples of set B, wherein plurality of patterns without a substantially distinct border is centred, or decentred, within of the spectacle lens.

The spectacle lens of one or more of the above claim examples of set B, wherein the total surface area of the plurality of non-refractive features occupies between 5 percent and 15 percent, or between 7.5 percent and 20 percent, or between 12.5 percent and 25 percent of the total surface area of the spectacle lens.

The spectacle lens of one or more of the above claim examples of set B, wherein the plurality of non-refractive features are configured to be within the central 10 mm, or central 15 mm, or central 20 mm, or central 30 mm, of the spectacle lens.

The spectacle lens of one or more of the above claim examples of set B, wherein region outside the central 30 mm, or outside the central 35 mm, or outside the central 40 mm, of the spectacle lens is substantially devoid of the non-refractive features.

The spectacle lens of one or more of the above claim examples of set B, wherein the plurality of non-refractive features is applied on the front surface, or the back surface, or both front and back surfaces.

The spectacle lens of one or more of the above claim examples of set B, wherein the plurality of non-refractive features is applied within the matrix of the contact lens.

The spectacle lens of one or more of the above claim examples of set B, wherein the substantially opaque border or feature is configured such that it absorbs at least 80 percent, at least 90 percent, or at least 99 percent of light incident on the substantially opaque border or feature.

The spectacle lens of one or more of the above claim examples of set B, wherein the total light transmittance through the optic zone is between 85 percent and 90 percent, or between 90 percent and 95 percent, or between 92.5 percent and 97.5 percent, or between 85 percent and 99 percent of the total light transmittance through the optic zone of a similar single vision lens devoid of the non-refractive features.

The spectacle lens of one or more of the above claim examples of set B, wherein the plurality of non-refractive features is configured at least in part to be sensitive to polarisation of the incident light.

The spectacle lens of one or more of the above claim examples of set B, wherein the plurality of non-refractive features are activated and turn opaque, at least in part, when the incident light is linearly, or circularly, or elliptically polarised.

The spectacle lens of one or more of the above claim examples of set B, wherein the plurality of non-refractive features are activated and turn opaque, at least in part, when the incident light is coming from a LCD or a LED or an OLED monitor screen, TV screen, tablet screen, or mobile screen or a screen of similar electronic devices.

The spectacle lens of one or more of the above claim examples of set B, wherein the plurality of non-refractive features is configured at least in part to be electronically tuneable.

The spectacle lens of one or more of the above claim examples of set B, wherein the non-refractive features are configured such that the material characteristics are spectrally sensitive to certain visible wavelengths between 420 to 760 nm, inclusive.

The spectacle lens of one or more of the above claim examples of set B, wherein the lens is capable of providing the wearer with adequate visual performance that is substantially similar to that obtained with a single vision lens devoid of non-refractive features.

The spectacle lens of one or more of the above claim examples of set B, when tested on a model eye configured with a distance refractive error substantially matching the base prescription, provides an on-axis modulation transfer function, for at least one pupil between 3 mm and 6 mm inclusive, and at least one wavelength 420 nm to 760 nm inclusive, which is substantially equivalent to that obtained with a single vison spectacle lens that is devoid of the non-refractive features.

The spectacle lens of one or more of the above claim examples of set B, when tested on a model eye configured with a distance refractive error substantially matching the base prescription, provides an off-axis wide-field modulation transfer function, for at least one pupil between 3 mm and 6 mm inclusive, and at least one wavelength 420 nm to 760 nm inclusive, which is substantially equivalent to that obtained with a single vison spectacle lens that is devoid of the non-refractive features.

The spectacle lens of one or more of the above claim examples of set B, wherein the wide-field of the retina includes at least 5°, or 10°, or 15°, or 20°, or 25°, or 30°, of the visual field.

The spectacle lens of one or more of the above claim examples of set B, when tested on a model eye configured with a distance refractive error substantially matching the base prescription, provides substantial correction of the distance refractive error of the eye and results in artificial edges, or spatial luminous contrast profiles, spread across the wide-field of the retina of the model eye.

The spectacle lens of one or more of the above claim examples of set B, when tested on a model eye configured with a distance refractive error substantially matching the base prescription, in various decentration positions to emulate eye movements of the wearer, to provide a temporal variation of the artificial edges, or spatial luminous contrast profiles, spread across the wide-field of the retina of the model eye.

The spectacle lens of one or more of the above claim examples of set B, wherein the model eye is a schematic, a physical, or a bench-top model eye.

The spectacle lens of one or more of the above claim examples of set B, when tested on a bench-top, or a physical, model eye configured with a distance refractive error substantially matching the base prescription, results in a substantial correction of the distance refractive error of the model eye.

The spectacle lens of one or more of the above claim examples of set B, wherein the retina of the bench-top, or physical, model eye comprising a camera with, a charge coupled device, or a complementary metal oxide sensor, is configured to capture images of a visual scene projected through the model eye corrected with the spectacle lens.

The spectacle lens of one or more of the above claim examples of set B, wherein the images captured by the retina of the model eye serves as an input stream for the virtual retinal simulator comprising at least one of the three image processing steps disclosed herein: (a) spatiotemporal filtering of the input stream of images resulting in a band-pass current, (b) an instantaneous non-linear contrast gain control using variable feedback gate shunt conductance, and (c) a discrete set of noisy integrate-and-fire cell models, resulting in spike trains depicting ganglion cell activity.

The spectacle lens of one or more of the above claim examples of set B, wherein the plurality of non-refractive regions are configured to provide an increase in the retinal ganglion cell activity compared to that obtained with a single vison spectacle lens that is devoid of the non-refractive features.

The spectacle lens of one or more of the above claim examples of set B, wherein the retinal ganglion cell activity, gauged as mean retinal spike rate integrated over a certain time frame, is at least 1.25 times, 1.5 times, 1.75 times, 2 times, 2.25 times, 2.5 times, 2.75 times, 3 times of the retinal ganglion cell activity of a single vison spectacle lens that is devoid of the non-refractive features.

The spectacle lens of one or more of the above claim examples of set B, the certain time frame over which the mean retinal spike rate is integrated may be at least 1 second, or at least 3 seconds, or at least 10 seconds, or at least 30 seconds, or at least 60 seconds, or at least 120 seconds, or at least 180 seconds.

The spectacle lens of one or more of the above claim examples of set B, wherein the retinal ganglion cell activity, or non-stationarity in neural response, gauged as mean retinal spike rate, is observed in on-centre/off-surround retinal field, or on-surround/off-centre retinal field, or both.

The spectacle lens of one or more of the above claim examples of set B, wherein the function describing the overall retinal ganglion cell activity at the retina of the model eye, or non-stationarity in neural response, gauged in terms of mean retinal spike rate as a function of time, follows a non-linear, or aperiodic, or a sinusoidal, or quasi-sinusoidal, rectangular-wave, quasi rectangular-wave, square-wave, quasi square-wave, or a non-monotonic, pattern depicting a temporal variation in the overall retinal ganglion cell activity.

The spectacle lens of one or more of the above claim examples of set B, wherein the plurality of non-refractive regions provide at least one of slowing, retarding, or preventing myopia progression, measured by change in axial length or distance refractive error of the eye.

The spectacle lens of one or more of the above claim examples of set B, where in the spectacle lens provides, at least in part, adequate foveal correction for the refractive error of the eye, and the non-refractive features provide, at least in part, a time-varying and/or spatially variant stop signal to reduce the rate of myopia progression.

The spectacle lens of one or more of the above claim examples of set B, wherein the effect of at least one of slowing, retarding, or preventing myopia progression is maintained across at least 12, 24, 36, 48, or 60 months of lens wear.

The spectacle lens of one or more of the above claim examples of set B, wherein peripheral region is devoid of the plurality of substantially opaque features.

The spectacle lens of one or more of the above claim examples of set B, wherein the non-refractive features are applied using pad-printing, laser etching, photo-etching, or laser printing.

The spectacle lens of one or more of the above claim examples of set B combined with one or more of the contact lens claim examples of Set A constitute additional embodiments.

1-32. (canceled)

33. An ophthalmic lens for an eye, the ophthalmic lens comprising a front surface, a back surface, an optical centre, and an optical zone about the optical centre, wherein:
the optical zone comprises a base prescription for the eye, and at least one region with a plurality of low transmission non-refractive features, providing a total light transmittance;
the base prescription comprises a spherical correction, an astigmatic correction, or a spherical and an astigmatic correction;
the plurality of low transmission non-refractive features each absorb at least 80% of light incident on the low transmission non-refractive features; and
the total light transmittance through the optical zone is between 85 percent and 99 percent of the total light transmittance through the optical zone of a similar single vision lens with the base prescription and devoid of the plurality of low transmission non-refractive features.

34. The lens of claim 33, wherein the plurality of low transmission non-refractive features comprise of a plurality of borders forming a plurality of apertures; and wherein the plurality of apertures include at least 3 apertures; and wherein each aperture circumscribes a substantially transparent region comprising the base prescription for the eye; and wherein a shape of each aperture is at least one of the following: circular, elliptical, oval, triangular, rectangular, square, pentagonal, or hexagonal, or octagonal, or any other regular polygon, or an irregular polygon, or a random shape; and wherein a surface area of the circumscribed transparent region of at least one of the plurality of apertures is between 0.25 sq mm and 7.5 sq mm; and wherein the plurality of apertures are configured in a circular, hexagonal, radial, spiral, regular, irregular, or random arrangement; and wherein a width of the border of any of the plurality of apertures is between 5 μm to 250 μm such that the border remains substantially non-diffractive.

35. The lens of claim 33, wherein each of the low transmission non-refractive feature has a width between 5 μm to 250 μm, is substantially non-diffractive, and is configured to be at least one of the following: dot-like, arc-like, or a straight line, a zig-zag line, a curvilinear line, or a striae; and wherein the plurality of the low transmission non-refractive features are arranged in at least one pattern; wherein the at least one pattern includes at least: a moiré pattern, a curvilinear pattern, a spoke wheel pattern, a dot-like pattern, a Memphis pattern, a rectangular grid pattern, a regular pattern, an irregular pattern, a hexagonal pattern, a spiral pattern, a swirl pattern, a radial pattern, an array of lines, a zig-zag, an interference pattern, a random pattern with dots, a random pattern with straight lines, a random pattern with non-circular dots, a random pattern with curvilinear lines, a random pattern with arcs, a random pattern with zig-zag lines or a random pattern.

36. The lens of claim 33, wherein a total surface area of the plurality of low transmission non-refractive features occupies between 2.5 percent and 15 percent of a total surface area of the optical zone; and the plurality of low transmission non-refractive features are configured to be within a central 5 mm of the optical zone; and a region outside a central 6 mm of the optical zone is substantially devoid of the non-refractive features.

37. The lens of claim 33, wherein the plurality of low transmission non-refractive features are applied at least in one of the locations: on the front surface, on the back surface, or within a material of the lens; and wherein the plurality of low transmission non-refractive features are configured to be at least one of the following: opaque, translucent, reflective, spectrally sensitive, polarisation sensitive, or absorbent.

38. The lens of claim 37, wherein the low transmission non-refractive features are configured such that the material characteristics are spectrally sensitive to certain visible wavelengths between 420 to 760 nm, inclusive; and wherein the plurality of low transmission non-refractive features are at least in part electronically tuneable, and are activated at least in part, when incident light on the lens is linearly, or circularly, or elliptically polarised.

39. The lens of claim 38, wherein the lens is configured to provide, when tested on a schematic, bench-top, or a physical, model eye configured with a distance refractive error substantially matching the base prescription, an on-axis, or an off-axis wide-field, optical performance, for at least one pupil between 3 mm and 6 mm inclusive, and at least one wavelength 420 nm to 760 nm inclusive, which is within 5% variation of that obtained with a single vison lens that is devoid of the low transmission non-refractive features; wherein the optical performance is gauged as modulation transfer function as a function of spatial frequencies; and wherein the off-axis wide-field includes at least 5 degrees of a visual field of the model eye.

40. The lens of claim 39, wherein the lens is configured to provide, when tested on the schematic, bench-top, or physical, model eye, a substantial correction of the distance refractive error of the model eye and an increase in spike trains depicting retinal ganglion cell activity; wherein the retina of the model eye is configured to capture images of a visual scene projected through the model eye corrected with the lens and wherein the captured images serve as an input stream for a virtual retinal simulator comprising at least one of the image processing: (a) spatiotemporal filtering of the input stream of images resulting in a band-pass current, (b) an instantaneous non-linear contrast gain control using variable feedback gate shunt conductance, and (c) a discrete set of noisy integrate-and-fire cell models, resulting in spike trains depicting retinal ganglion cell activity; wherein the lens provides the increase in spike trains depicting the retinal ganglion cell activity compared to that obtained with a single vison lens that is devoid of the low transmission non-refractive features.

41. The lens of claim 40, wherein the captured images of the visual scene result in artificial edges, or spatial luminous contrast profiles, spread across a wide-field of a retina of the model eye.

42. The lens of claim 41, wherein the captured images of the visual scene comprise captured images at various decentration positions to emulate one of the following: on-eye movement of the lens; eye movements of the wearer, or combination thereof, provides a temporal variation of the said artificial edges, or spatial luminous contrast profiles, spread across the wide-field of the retina of the model eye.

43. The lens of claim 42, wherein the retinal ganglion cell activity, gauged as mean retinal spike rate, or a non-stationarity in neural response, is observed in at least one of on-centre/off-surround retinal field and on-surround/off-centre retinal field.

44. The lens of claim 43, wherein the retinal ganglion cell activity, or the non-stationarity in neural response, gauged as mean retinal spike rate integrated over a certain time frame, is at least 1.5 times of the retinal ganglion cell activity of a single vison lens that is devoid of the low transmission non-refractive features; and wherein the certain time frame over which the mean retinal spike rate is integrated over is at least 1 second.

45. The lens of claim 44, wherein the retinal ganglion cell activity, or the non-stationarity in neural response, gauged in terms of mean retinal spike rate as a function of time, follows one of the following: a non-linear, an aperiodic, a sinusoidal, a quasi-sinusoidal, a rectangular-wave, a quasi-rectangular-wave, a square-wave, a quasi-square-wave, or a non-monotonic, pattern depicting a temporal variation in the overall retinal ganglion cell activity.

46. The lens of claim 45, wherein the lens provides visual performance that is substantially similar to that obtained with a single vision lens devoid of the low transmission non-refractive features.

47. The lens of claim 46, wherein the lens provides at least one of slowing, retarding, or preventing myopia progression, measured by change in axial length or distance refractive error of the eye over time; wherein the measurement of change over time is considered after at least 6, 12, or 24 months of lens wear.

48. The lens of claim 47, wherein the non-refractive features were applied using pad-printing, laser etching, photo-etching, or laser printing.

49. The lens of claim 48, wherein the plurality of non-refractive features are activated and turn opaque, at least in part, when the incident light is coming from a LCD or a LED or an OLED monitor screen, TV screen, tablet screen, or mobile screen or a screen of similar electronic devices.

50. The lens of claim 49, wherein the off-axis wide-field includes at least 15 degrees of a visual field of the model eye.

51. The lens of claim 42, wherein the on-eye movement is within 1 mm of a well-centred on-eye lens position and in one of the following directions: horizontal, oblique, vertical or combinations thereof.

52. An ophthalmic lens for an eye, the ophthalmic lens comprising a front surface, a back surface, an optical centre, and an optical zone about the optical centre, wherein:
the optical zone comprises a base prescription for the eye, and at least one region with a plurality of low transmission non-refractive features, providing a total light transmittance;
the base prescription comprises a spherical correction for myopia, with or without an astigmatic correction; and
the total light transmittance through the optical zone is at least 85 percent of the total light transmittance through a like optical zone of a single vision lens with the base prescription and devoid of the plurality of low transmission non-refractive features.
